(19) United States
(12) Patent Application Publication
(10) Pub. No.: US 2022/0268785 A1
(43) Pub. Date: Aug. 25, 2022

(54) FACILE SAMPLE PREPARATION FOR QUANTITATIVE SINGLE-CELL PROTEOMICS

(71) Applicants: Northwestern University, Evanston, IL (US); Battelle Memorial Institute, Richland, WA (US)

(72) Inventors: Tujin Shi, Richland, WA (US); Huiping Liu, Chicago, IL (US); Chia-Feng Tsai, West Richland, WA (US); David Scholten, Chicago, IL (US); Reta Birhanu Kitata, Richland, WA (US); Carolina Reduzzi, Evanston, IL (US)

(21) Appl. No.: 17/651,896

(22) Filed: Feb. 21, 2022

Related U.S. Application Data
(60) Provisional application No. 63/151,537, filed on Feb. 19, 2021.

(51) Int. Cl. G01N 33/68

(52) U.S. Cl. CPC G01N 33/6848; G01N 2570/00; G01N 2440/00; G01N 2560/00

(57) ABSTRACT

Disclosed are compositions and methods for performing a proteomic analysis. Particularly disclosed are compositions and methods for preparing a sample for quantitative single-cell proteomics.

### CROSS-REFERENCE TO RELATED APPLICATIONS

This application claims benefit of priority to U.S. Provisional Application No. 63/151,537, filed Feb. 19, 2021, the content of which is incorporated herein by reference in its entirety.

### STATEMENT REGARDING FEDERALLY SPONSORED RESEARCH OR DEVELOPMENT

This invention was made with government support under UG3CA256967 and CA223715 awarded by the National Institutes of Health and W81XWH-16-1-0021 awarded by the Department of Defense Breast Cancer Research Program (DOD BCRP). The government has certain rights in the invention.

### BACKGROUND AND SUMMARY

The field of the invention relates to compositions and methods for performing a mass spectrometry-based proteomic analysis of proteome and target proteins with genetic alterations and post-translational modifications. In particular, the field of the invention relates to compositions and methods for preparing a sample for one-pot quantitative proteomics near and at single-cell and subcellular levels with minimal protein loss and maximal protein recovery during processing, and for global proteome profiling and targeted analyses of peptide variants and mutations in normal and abnormal cells (such as cancer) via mass spectrometry.

In one aspect of the current disclosure, methods for performing proteomic analysis on a sample are provided. In some embodiments, the methods comprise treating the sample with a non-ionic surfactant, performing mass spectrometry on the treated sample, and detecting proteins in the treated sample. In some embodiments, the non-ionic surfactant is an alkyl glucoside. In some embodiments, the non-ionic surfactant is an alkyl diglucoside. In some embodiments, the non-ionic surfactant is an alkyl maltoside. In some embodiments, the non-ionic surfactant is octyl-maltoside, decyl-maltoside, dodecyl-maltoside, or tetradecyl-maltoside. In some embodiments, the non-ionic surfactant is n-dodecyl-β-D-maltoside (In some embodiments, the concentration is 0.01% to 0.02%. In some embodiments, the concentration is 0.015%. In some embodiments, the method results in at least about a 20-fold enhancement in the mass spectrometry signal from the sample when compared to a sample not treated with the non-ionic surfactant. In some embodiments, the detected protein comprises an amino acid sequence of a peptide of Tables 2-7.

In another aspect of the current disclosure, methods for performing proteomic analysis on a single cell are provided. In some embodiments, the methods comprise isolating a single cell to prepare a sample, treating the sample with a non-ionic surfactant, performing mass spectrometry on the treated sample and detecting proteins in the treated sample. In some embodiments, the non-ionic surfactant is an alkyl glucoside. In some embodiments, the non-ionic surfactant is an alkyl diglucoside. In some embodiments, the non-ionic surfactant is an alkyl maltoside. In some embodiments, the non-ionic surfactant is octyl-maltoside, decyl-maltoside, dodecyl-maltoside, or tetradecyl-maltoside. In some embodiments, the non-ionic surfactant is n-dodecyl-β-D-maltoside (DDM). In some embodiments, the concentration of the non-ionic surfactant is 0.005% to 0.1%. In some embodiments, the concentration is 0.01% to 0.02%. In some embodiments, the concentration is 0.015%. In some embodiments, the method results in at least about a 20-fold enhancement in the mass spectrometry signal from the sample when compared to a sample not treated with the non-ionic surfactant. In some embodiments, the detected protein comprises an amino acid sequence of a peptide of Tables 2-7.

### BRIEF DESCRIPTION OF THE DRAWINGS

FIG. 1. Schematic diagram of the SOP-MS workflow. a, Single cells or small numbers of cells are sorted either by fluorescence activated cell sorting (FACS) or laser capture microdissection (LCM) and collected into single PCR tube or a 96-well PCR plate. After FACS isolation, the sorted cells are subjected to centrifugation at 1000 g for 10 min to ensure them at the bottom of the PCR tube or 96-well PCR plate. For LCM, the dissected tissue voxels are catapulted into a 5 μL water droplet on the PCR tube cap, followed by centrifugation at 1000 g for 10 mins. b, For cell lysis, a cell lysis buffer containing 0.2% (w/v) n-Dodecyl β-D-maltoside (DDM) is added to the PCR tube or 96-well PCR plate followed by incubation at 75° C. for 1 h. Sample are then subjected to reduction and alkylation (these two steps are optional). Small amounts of trypsin are used for overnight digestion: 2 ng for single cells and 5 ng for 50-100 cells. c, Prior to LC-MS analysis, the cap of the PCR tube is removed and then the tube is inserted into a sample vial to avoid transfer loss. The 96-well cap matt is used to cover the 96-well plate for automatic injection without sample transfer. Samples are analyzed by standard LC-MS platforms for quantitative proteomic analysis. The freely available open-source MaxQuant software is used for label-free quantification. d, Number of unique peptides and protein groups identified by MS/MS only for 0.2 ng of AML cell lysate digests equivalent to 2 cells (three biological replicates per condition) without and with 0.015% DDM. e, The total extracted ion chromatogram (XIC) peak area for 0.2 ng of AML cell lysate digests equivalent to 2 cells (three biological replicates per condition) without and with 0.015% DDM.

FIG. 2. SOP-MS analysis of LCM-dissected mouse uterine tissue. a, Number of unique peptides and protein groups identified by the MS/MS spectra only (without MBR) from three biological replicates per cell type (luminal epithelial and stroma) and three blanks. The LCM tissue size: 100 μm (length)×100 μm (width)×10 μm (thickness). Each LCM-dissected tissue sample is close to ˜20 cells. b, Pairwise correlation of log₁₀-transformed protein LFQ intensities between any two replicates. Pearson correlation coefficients were color coded as shown on the scale at the bottom. c, Unsupervised PCA analysis based on label-free quantification of proteins expressed in luminal epithelial and stroma cells. d, Volcano plot of proteins differentially expressed between the two cell types from three biological replicates per cell type.

FIG. 3. SOP-MS analysis of single MCF10A cells sorted by FACS. a, Number of unique peptides and protein groups identified by MS/MS only and the combined MS/MS and MBR from single MCF10A cells without and with the addition of 0.015% DDM (three biological replicates per condition; P<0.05 between without and with DDM). b, Total number of unique peptides and protein groups identified by MS/MS only and the combined MS/MS and MBR across all three biological replicates without and with the addition of 0.015% DDM. c, Venn diagram showing the number of protein groups identified from each of 3 single MCF10A cells with the addition of 0.015% DDM by the combined MS/MS and MBR. d, The summed total XIC peak area for all quantifiable peptides from single MCF10A cells (three biological replicates per condition; P<0.05 between without and with DDM). e, Total number of unique peptides and protein groups identified by the MS/MS spectra alone from all three biological replicates using three common search tools (MaxQuant, MSGF+, and MSFragger). f, Pairwise correlation of protein LFQ intensities between any two replicates with the Pearson correlation coefficient. g, Distribution of protein abundance for all proteins identified from single MCF10A cells and 10 ng MCF10A cell lysate digests. Library was built with 10 ng MCF10A cell lysate digests.

FIG. 4. Validation of SOP-MS for quantitative single-cell proteomics analysis. a, Number of unique peptides and protein groups identified by the MS/MS spectra alone from 3 single MCF7 cells and 4 single MCF10A cells (from newly cultured MCF10A cells) sorted by FACS. b, Venn diagram showing the number of protein groups identified from each of single MCF7 or MCF10A cells. c, Pairwise correlation of log₁₀-transformed protein LFQ intensities between any two replicates from the 3 singe MCF7 cells and the 4 single MCF10A cells. Pearson correlation coefficients were color coded as shown on the scale at the bottom. Half of sample injection was used for analysis of single MCF7 cells (i.e., 0.5 single MCF7 cells for MS analysis).

FIG. 5. SOP-MS analysis of single cells derived from a PCDX model. a, Schematic workflow of SOP-MS analysis of single cells derived from a PCDX model. CTCs from a breast cancer patient (NU-205) were isolated and implanted into NSG mouse mammary fat pads to generate the PCDX-205 mouse. The PCDX was verified (FIG. 10) and transduced to express Luc2-tdTomato (L2T). Labeled primary PCDX and lungs were harvested for tissue dissociation and single cell sorting. L2T⁺ single cells from the primary tumor and lung metastases were collected individually into single well of a 96-well PRC plate for SOP-MS analysis. b, Total number of unique peptides and protein groups identified by the combined MS/MS and MBR across all 10 single lung metastatic cells or 10 single primary tumor cells, and the number of protein groups identified by the combined MS/MS and MBR for each single lung metastatic cells or single primary tumor cells. c, Heatmap showing the total XIC peak area for each protein group identified by the MaxQuant MBR from either the 10 single primary tumor cells or the 10 single lung metastatic cells. d, PCA analysis based on label-free quantification of proteins expressed in single cells from primary tumor and lung metastasis (10 single cells for each cell type). e, Heatmap showing 18 differentially expressed proteins between single cells from primary tumor and lung metastasis. f, Bar chart for pathway annotation. The bars represent the annotated pathways within proteins significantly expressed between two types of single cells. g, Box plots showing the normalized expression levels of VIM (left) and S100A9 (right) between single lung metastatic cells and single primary tumor cells by using SOP-MS (10 single cells per cell type). h, Immunohistochemistry (IHC) images of primary tumors and lung metastases, stained for VIM (top) and S100A9 (bottom). Arrows indicate representative, positive staining tumor cells. Scale bar=50 μm.

FIG. 6. Evaluation of sample recovery and processing reproducibility in single PCR tube with and without DDM. SRM-based targeted quantification of a mixture of heavy isotope-labeled phosphopeptide standards without DDM (Control) and with DDM (DDM). XIC (Counts) corresponds to the SRM signal for peptide standards. The P values were shown for each peptide between without and with DDM additive.

FIG. 7. Number of unique peptides and protein groups identified by MS/MS only for 0.1, 0.5, 1, 5 ng of tryptic peptides from lung cancer PC9 cell lysate digests (equivalent to 1, 5, 10, and 50 cells) between without and with 0.015% DDM. Clearly, DDM can significantly improve the number of identified peptides and proteins (three biological replicates per condition). The data have been newly generated by two different groups using two different MS instruments (low-end Q Exactive MS and the most advanced Lumos MS).

FIG. 8. Evaluation of SOP-MS performance for analysis of low mass inputs of MCF7 cell lysates (0-2.5 ng) from serial dilution. a. Number of unique peptides (left panel) and protein groups (right panel) identified from 0, 0.05, 0.25, 0.5 and 2.5 ng of MCF7 lysates with duplicates for each mass input. b. The number of unique peptides, protein groups, and Log 2 extracted ion chromatogram (XIC) area as a function of low mass inputs from MCF7 cell lysates (0, 0.05 ng≈0.5 cells, 0.25 ng≈2.5 cells, 0.5 ng≈5 cells). c. Correlations of Log 2LFQ between duplicates for each mass input. d. Correlations of Log₂LFQ between any two replicates out of 5 replicates for 5 ng of MCF7 cell lysates.

FIG. 9. LCM dissected small sections from mouse uterine tissues. a. Image of three biological replicates for each tissue region (luminal epithelia and stroma) with a size of 100 μm in diameter and 10 μm in thickness (equivalent to ˜20 cells). b. PCA analysis for identification of cell type-specific proteins. Blue dots indicate the proteins relevant to extracellular matrix receptor interactions and cell adhesion, which are specific to stroma region. c. Enriched proteins in the luminal epithelial region are relevant to EGF-like domain, immunoglobulin domain, and transmembrane domain.

FIG. 10. CTC-205 PDX model workflow a. CTCs isolated from blood of a breast cancer patient (NU-205) were confirmed to express cytokeratin (CK) and HER2 and to be negative for CD45 using the CellSearch platform analysis. b. CTCs from a breast cancer patient (NU-205) were enriched by depletion of CD45⁺ PBMCs and implanted into NSG mouse mammary fat pads to generate the breast tumor xenograft PCDX-205. Flow cytometry profiles of the PCDX-205 show a negative expression of mouse stroma marker H2K^{d} and proportional positive expression for human epithelial tumor markers EpCAM, HER2, CD44 and EGFR. c. PCDX-205 cells were transduced with lentivirus to express fluorescent L2T, and re-implanted in NSG mice. d. Tumors and lungs of PCDX-205-bearing mice were dissociated, and L2T⁺ single cells from the tumor and lungs were sorted into a 96-well PCR plate. Cells were sorted based on tdTomato⁺ expression. e. Single cells were analyzed by SOP-MS.

FIG. 11. Performance comparison of SOP-MS with nanoPOTS-MS for analysis of single MCF10A cells. a, Number of unique peptides and protein groups identified by the MS/MS spectra alone from 4 single MCF10A cells sorted by FACS for each method. b, Venn diagram showing the number of total protein groups identified from each method.

FIG. 12. Initial evaluation of multiplexed proteomic analysis of 9 single MCF10A cells sorted by FACS by using the combined TMT-based BASIL and SOP-MS. a. TMT-11 channel assignment (TMT-labeled sample channels for single MCF10A cells and a boosting channel with 10 ng of MCF10A cell lysate digests). b. Signal distribution of all TMT-11 channels. c. Number of quantifiable peptides and protein groups identified from single MCF10A cells. d. Pearson correlation for all 9 single MCF10A cells analyzed by SOP-MS with a median correlation of ˜0.95. Half of sample injection was used for MS analysis (i.e., 0.5 single MCF10A cells for each channel).

FIG. 13. Mass spectrometry profiles of single amino acid variant (SAAV) sites for mutation peptides derived from proteins KRAS and SLC37A4 in the PANC-1 cancer cell line using highly specific selected reaction monitoring (SRM) assays. (a) Variant peptide LVVVGADGVGK (SEQ ID NO: 1) (variant peptide G12D) from KRAS and canonical peptide LVVVGAGGVGK (SEQ ID NO: 2) (wildtype) from KRAS. (b) Variant peptide FVSGVLSDQMSAR (SEQ ID NO: 3) from SLC37A4. The endogenous peptides were confirmed by matching their corresponding heavy internal standards in the retention time and the SRM peak patterns. The top panel shows the SRM signal for endogenous peptides; the bottom panel shows the SRM signal for heavy internal standards (¹³C6, ¹⁵N2 on the C-terminal K or R). IS, internal standard.

FIG. 14. Mass spectrometry profiles of single amino acid variant (SAAV) sites for mutation peptides derived from SPOP in the prostate cancer cell line using highly specific SRM assays. (A) Canonical peptide VNPKGLDEESKDYLSLYLLLVSCPKSEVR (SEQ ID NO: 4) and variant peptide VNPKGLDEESKDYLSLNLLLVSCPKSEVR (SEQ ID NO: 5) (variant peptide Y87N) from SPOP. (b) Canonical peptide AKFKFSILNAKGEETKAMESQR (SEQ ID NO: 6) and variant peptide AKCKFSILNAKGEETKAMESQR (SEQ ID NO: 7) (variant peptide F102C) from SPOP. The endogenous peptides were confirmed by matching their corresponding heavy internal standards in the retention time and the SRM peak patterns. The top panel shows the SRM signal for endogenous peptides; the bottom panel shows the SRM signal for heavy internal standards (¹³C6, ¹⁵N2 on the C-terminal R). IS, internal standard.

### DETAILED DESCRIPTION

### General Definitions

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization”, as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### Methods for Performing Proteomic Analysis on a Sample

The field of single-cell proteomic analysis of regular-size mammalian cells remains highly challenging, primarily due to technical difficulties in effective sampling and processing. In particular, protein loss due to adsorption remains a major pitfall of any small sample size proteomics methodology, e.g., single-cell proteomics. To alleviate the shortcomings of existing proteomic approaches, the inventors developed a broadly adoptable MS method for quantitative single-cell proteomics for both label-free and tandem mass tag (TMT) labeling analysis. This method capitalizes on surfactant-assisted one-pot (single tube or multi-well plate) processing coupled with MS (termed SOP-MS) for greatly reducing the surface adsorption losses, thus, improving detection sensitivity for MS analysis of single cells and mass-limited clinical specimens. Critically, the inventors discovered that the use of alkly glucosides, e.g., n-Dodecyl β-D-maltoside (DDM), maximizes recovery for quantitative small sample proteomics by greatly reducing surface adsorption losses.

As used herein, “n-Dodecyl β-D-maltoside (DDM)” refers to a compound with a formula:

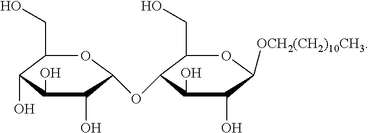

Accordingly, in one aspect of the current disclosure, methods for performing proteomic analysis on a sample are provided. In some embodiments, the methods comprise treating the sample with a non-ionic surfactant, performing mass spectrometry on the treated sample, and detecting proteins in the treated sample.

As used herein, “detecting” refers to determining the presence of a protein, or a portion thereof in a sample. In some embodiments, detecting comprises determining the presence of a peptide, wherein a protein comprises said peptide; Thus, detection of the peptide may confirm the presence of a protein in the sample. For example, detection of the peptide with sequence consisting of SEQ ID NO:1, which is an oncogenic variant derived from KRAS, indicates the presence of KRAS, i.e., oncogenic mutant KRAS, in a sample. Similarly, detection of a variety of proteins can be accomplished by detecting a peptide with an amino acid sequence of a peptide found in Tables 2-7. In some embodiments, the peptide found in a table is described by a variant, or “non-canonical” amino acid, inserted amino acid, or deleted amino acid. It will be apparent to one of skill in the art that such information can be used to describe peptides that are detected by the disclosed methods by referring to the canonical sequence of the given protein and making the change to the amino acid sequence that is indicated in the table. In some embodiments, detection is automated and does not require the use of the human mind.

In some embodiments, the non-ionic surfactant is an alkyl glucoside. In some embodiments, the non-ionic surfactant is an alkyl diglucoside. In some embodiments, the non-ionic surfactant is an alkyl maltoside. In some embodiments, the non-ionic surfactant is octyl-maltoside, decyl-maltoside, dodecyl-maltoside, or tetradecyl-maltoside. In some embodiments, the non-ionic surfactant is n-dodecyl-β-D-maltoside (DDM). In some embodiments, the concentration of the non-ionic surfactant is 0.005% to 0.1%. In some embodiments, the concentration is 0.01% to 0.02%. In some embodiments, the concentration is 0.015%. In some embodiments, the method results in at least about a 20-fold enhancement in the mass spectrometry signal from the sample when compared to a sample not treated with the non-ionic surfactant. In some embodiments, the detected protein comprises an amino acid sequence of a peptide of Tables 2-7.

As used herein, “proteomic analysis” refers to any technique whereby the proteome, or a portion thereof, of a sample from a subject is sequenced. In some embodiments, sequencing comprises determining the amino acid sequence of proteins in a sample. In some embodiments, sequencing comprises determining a substantial portion of the amino acid sequences of proteins in a sample, e.g., sequencing 50% of the proteins, 60% of the proteins, 70% of the proteins, 80% of the proteins, 90% of the proteins, 95% of the proteins, or more than 95% of the proteins in a sample. In some embodiments, proteomic analysis comprises mass spectrometry. In some embodiments, proteomic analysis comprises liquid chromatography coupled to tandem mass spectrometry (LC-MS/MS or LC-MS²), which may, in some embodiments, includes high-performance liquid chromatography coupled to tandem mass spectrometry (HPLC-MS/MS).

Mass spectrometry is an analytical technique used to measure the mass-to-charge ratio (m/z or m/q) of ions. It is most generally used to analyze the composition of a physical sample by generating a mass spectrum representing the masses of sample components. The technique has several applications including identifying unknown compounds by the mass of the compound and/or fragments thereof determining the isotopic composition of one or more elements in a compound, determining the structure of compounds by observing the fragmentation of the compound, quantitating the amount of a compound in a sample using carefully designed methods (mass spectrometry is not inherently quantitative), studying the fundamentals of gas phase ion chemistry (the chemistry of ions and neutrals in vacuum), and determining other physical, chemical or even biological properties of compounds with a variety of other approaches.

A mass spectrometer is a device used for mass spectrometry, and it produces a mass spectrum of a sample to analyze its composition. This is normally achieved by ionizing the sample and separating ions of differing masses and recording their relative abundance by measuring intensities of ion flux. A typical mass spectrometer comprises three parts: an ion source, a mass analyzer, and a detector.

The kind of ion source is a contributing factor that strongly influences-what types of samples can be analyzed by mass spectrometry. Electron ionization and chemical ionization are used for gases and vapors. In chemical ionization sources, the analyte is ionized by chemical ion-molecule reactions during collisions in the source. Two techniques often used with liquid and solid biological samples include electrospray ionization (ESI) and matrix-assisted laser desorption/ionization (MALDI). Other techniques include fast atom bombardment (FAB), thermospray, atmospheric pressure chemical ionization (APCI), secondary ion mass spectrometry (SIMS), and thermal ionisation.

Liquid-chromatography-tandem-mass spectrometry (LC-MS/MS) has been introduced in clinical chemistry (Vogeser M., Clin. Chem. Lab. Med. 41 (2003) 117-126). Advantages of this technology are high analytical specificity and accuracy and the flexibility in the development of reliable analytical methods. In contrast to gas chromatography mass spectrometry (GC-MS) as the traditional mass spectrometric technology in clinical chemistry. LC-MS/MS has been shown to be a robust technology, allowing its application also in a large-scale routine laboratory setting.

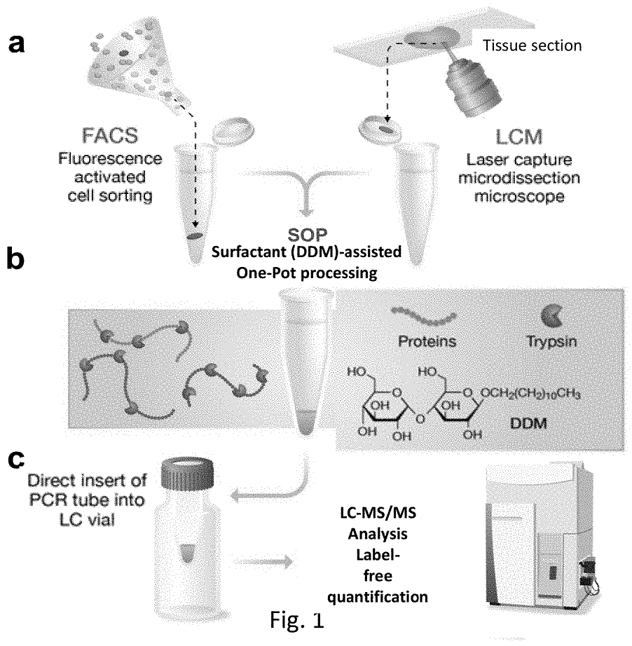

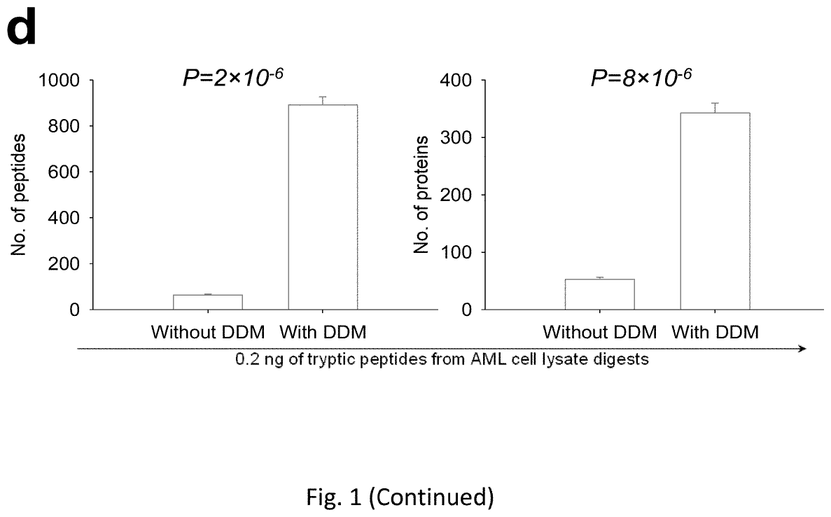

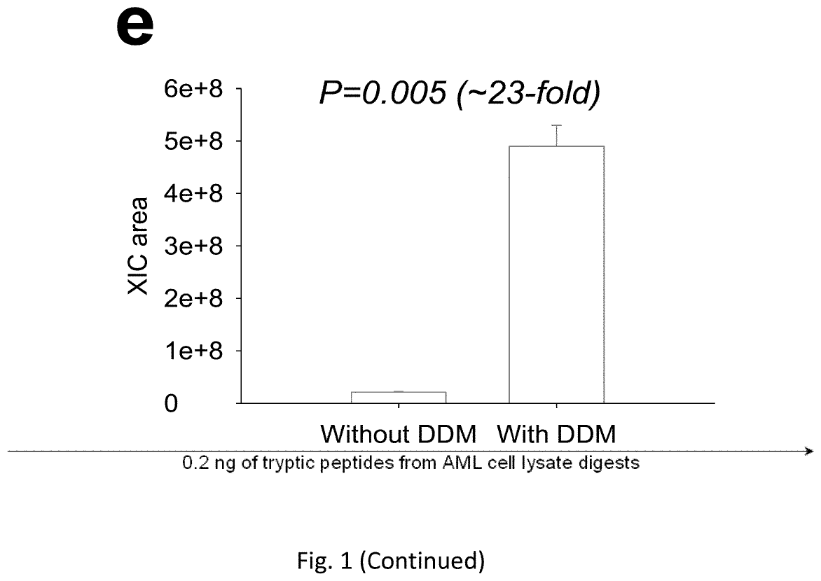

The inventors demonstrated that treating of samples for proteomic analysis, e.g., LC-MS/MS, with DDM decreases the loss of proteins due to adsorption to surfaces used in handling and preparing the samples, e.g., tubes, plates, etc. Therefore, inclusion of DDM in the preparation of samples for proteomic analysis, e.g., LC-MS/MS increases the mass spectrometry signal at least about 20-fold than without treatment with DDM (FIGS. 1d-e). As used herein, “mass spectrometry signal” refers to, in some embodiments, the number of detectable peptides or proteins. In the example above in FIGS. 1d-e, the method increased detection from 63 peptides in untreated samples to 891 peptides in DDM treated samples.

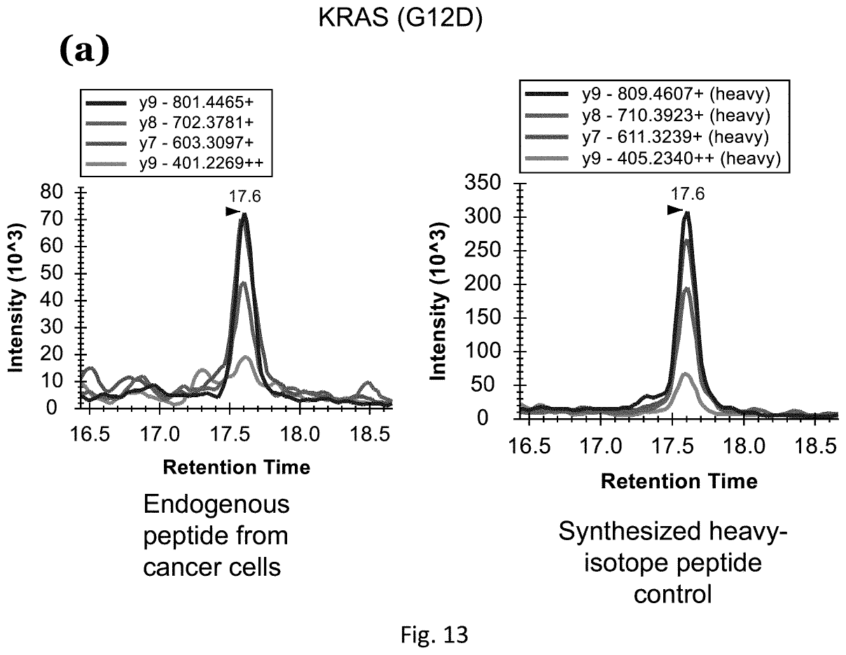

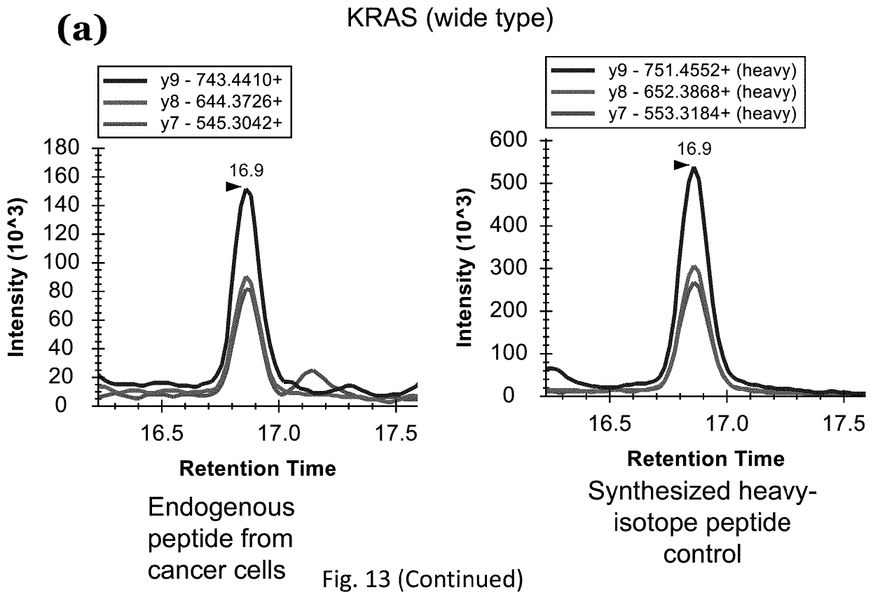

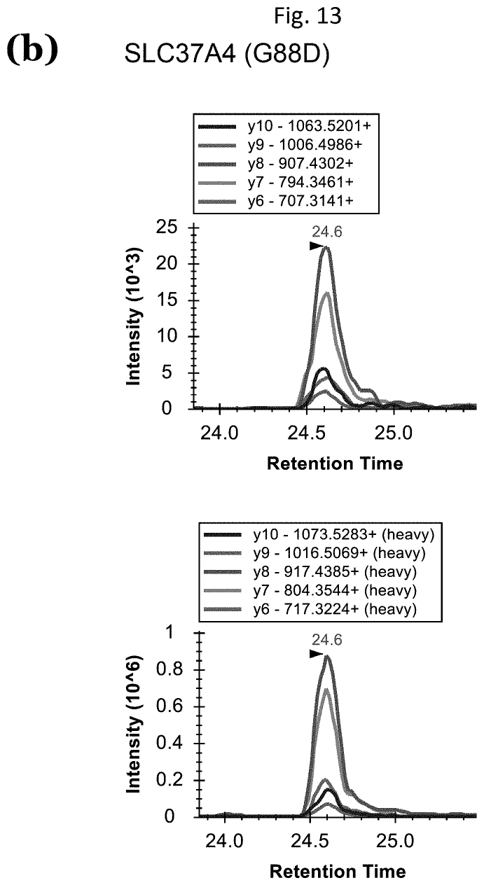

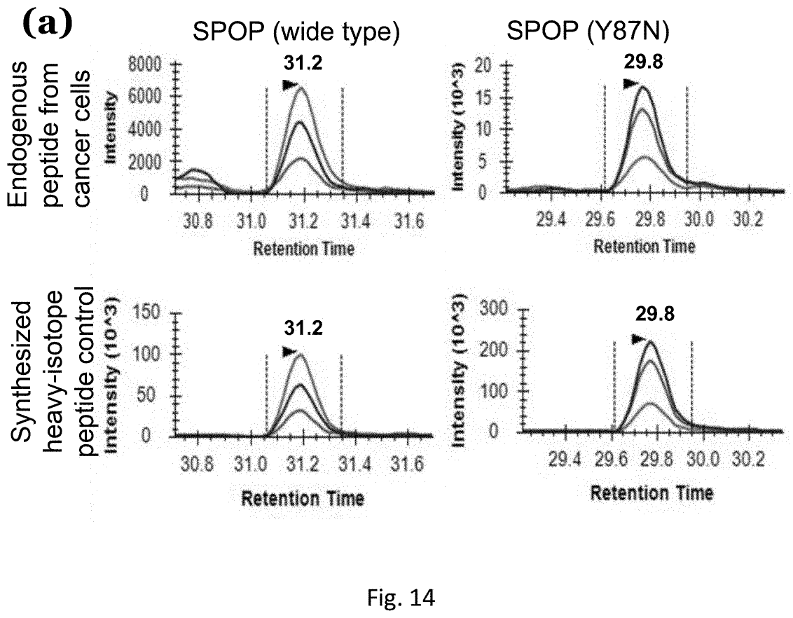

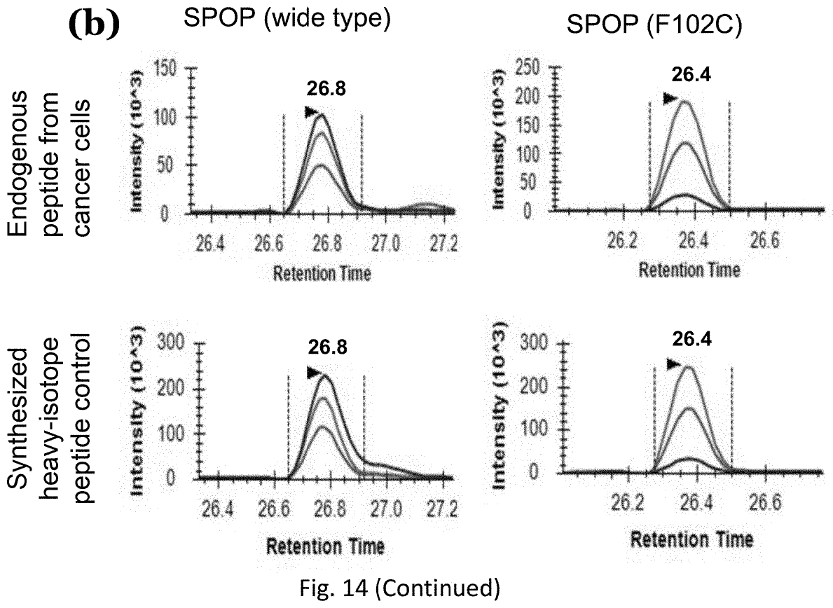

A key factor for development of single-cell proteomic assays is the ability to preserve the small amount of starting material derived from a sample consisting of one or a small number of cells. As used herein, a “small number of cells” is less than 50 cells, less than 40 cells, less than 30 cells, less than 20 cells, preferably less than 10 cells. Thus, the inventors demonstrated that treatment of small numbers of cell samples with DDM allows detection of protein variants, e.g., oncogenic variants (FIGS. 13 and 14).

Therefore, in some embodiments, methods for performing proteomic analysis on a single cell are provided. In some embodiments, the methods comprise isolating a single cell to prepare a sample, treating the sample with a non-ionic surfactant, and performing mass spectrometry on the treated sample. In some embodiments, the methods comprise isolating a single cell to prepare a sample, treating the sample with a non-ionic surfactant, performing mass spectrometry on the treated sample and detecting proteins in the treated sample. In some embodiments, the non-ionic surfactant is an alkyl glucoside. In some embodiments, the non-ionic surfactant is an alkyl diglucoside. In some embodiments, the non-ionic surfactant is an alkyl maltoside. In some embodiments, the non-ionic surfactant is octyl-maltoside, decyl-maltoside, dodecyl-maltoside, or tetradecyl-maltoside. In some embodiments, the non-ionic surfactant is n-dodecyl-β-D-maltoside (DDM). In some embodiments, the concentration of the non-ionic surfactant is 0.005% to 0.1%. In some embodiments, the concentration is 0.01% to 0.02%. In some embodiments, the concentration is 0.015%. In some embodiments, the method results in at least about a 20-fold enhancement in the mass spectrometry signal from the sample when compared to a sample not treated with the non-ionic surfactant. In some embodiments, the detected protein comprises an amino acid sequence of a peptide of Tables 2-7.

### EXAMPLES

The following Examples are illustrative and should not be interpreted to limit the scope of the claimed subject matter.

### Example 1

### Technical Field

The disclosed subject matter relates to a methodology breakthrough with a 20-fold improvement of sample recovery for mass spectrometry-based single-cell proteomic analyses.

### Abstract

Large numbers of cells are generally required for quantitative global proteome profiling due to the significant surface adsorption losses associated with sample processing. Such bulk measurement obscures important cell-to-cell variability (cell heterogeneity) and makes proteomic profiling impossible for rare cell populations, such as circulating tumor cells (CTCs) and early metastatic cells. Herein the inventors report a facile mass spectrometry (MS)-based single-cell proteomics method that capitalizes on a MS-compatible nonionic surfactant, n-Dodecyl-β-D-maltoside (DDM), for greatly reducing the surface adsorption losses by ˜20-fold for effective single-tube processing of single cells, thus significantly improving detection sensitivity for single-cell proteomic analysis. With standard MS platforms, the method allows for the first time precise, label-free, reliable quantification of hundreds of proteins from single human cells in a simple, convenient manner. When applied to a patient CTC-derived xenograft (PCDX) model, the method can reveal distinct protein signatures between primary tumor cells and early metastases to the lungs at the single-cell resolution. The approach paves the way for routine, precise quantitative single-cell proteomic analysis.

Applications

The disclosed subject matter has applications which may include, but are not limited to: (i) both global and targeted single-cell proteomics in all biomedical fields; (ii) elucidation of cellular heterogeneity across and within populations, especially rare populations of stem cells, circulating tumor cells, and early metastatic cells; (iii) potential applications to subcellular organelle proteomics, like nucleus, mitochondria, etc.; and (iv) 3D or 4D proteomic mapping of normal and pathological tissues at single cell resolution.

Advantages

The disclosed subject matter has advantages which may include, but are not limited to the following. There is no exisiting commercial services for single-cell proteomics due to technical barriers from surface adsorption losses duing sample processing. Previous two single-cell proteomic methods based on nanoPOTS-Lumos MS¹ and iPAD1-Lumos MS² require specific device and are extremely difficult for broad dissemination.

The disclosed breakthrough methodology based on the nonionic surfactant DDM additive increases 20-fold in sample recovery and compatible with standard mass-spectrometry for convenient commercialization. Moreover, the sample recovery and peptide analyses are similar to that with two previous methods on special devices. The broad applications of incoming single-cell proteomic analyses will bring unprecedented impact to the biological and medical field, including basic science, translational research, and clinical medicine.

### DESCRIPTION

To alleviate the shortcomings of existing proteomic approaches, the inventors have recently developed a facile, broadly adoptable MS method for precise quantitative single-cell proteomic analysis. This method capitalizes on surfactant-assisted one-pot processing coupled with MS (termed SOP-MS) for greatly reducing the surface adsorption losses, thus significantly improving detection sensitivity for MS analysis of single cells. SOP-MS was demonstrated to enable reliable quantification of hundreds of proteins from single cells with standard MS platforms. When it was applied to analyze two types of single cells isolated from patient CTC-derived xenografts (PCDXs): CTCs propagated in the mouse mammary fat pads with CSC properties (primary tumor cells) and their early micrometastases seeded to the lungs (lung micromets), SOP-MS not only allows for identification of protein signatures that can be leveraged for CTC characterization, but also facilitates elucidating heterogeneous alterations of metastatic tumor cells upon colonization of the lungs. Interestingly, the protein alterations in these cells are related to the selection pressure of anti-tumor immunity (e.g., neutrophils and innate immunity) for the transition from primary tumor CTCs to the early metastatic cells. These results demonstrate great potential of SOP-MS for broad applications in quantitative single-cell proteomics.

### REFERENCES

1. Zhu, Y. et al. Proteomic Analysis of Single Mammalian Cells Enabled by Microfluidic Nanodroplet Sample Preparation and Ultrasensitive NanoLC-MS. Angewandte Chemie-International Edition 57, 12370-12374, doi:10.1002/anie.201802843 (2018).
2. Shao, X. et al. Integrated Proteome Analysis Device for Fast Single-Cell Protein Profiling. Anal Chem 90, 14003-14010, doi:10.1021/acs.analchem.8b03692 (2018).
3. Zhu, Y. et al. Nanodroplet processing platform for deep and quantitative proteome profiling of 10-100 mammalian cells. Nat Commun 9, 882, doi:10.1038/s41467-018-03367-w (2018).
4. Shi, T. et al. Facile carrier-assisted targeted mass spectrometric approach for proteomic analysis of low numbers of mammalian cells. Commun Biol 1, 103, doi:10.1038/s42003-018-0107-6 (2018).
5. Zhang, P. et al. Carrier-Assisted Single-Tube Processing Approach for Targeted Proteomics Analysis of Low Numbers of Mammalian Cells. Anal Chem 91, 1441-1451, doi:10.1021/acs.analchem.8b04258 (2019).
6. Budnik, B., Levy, E., Harmange, G. & Slavov, N. SCoPE-MS: mass spectrometry of single mammalian cells quantifies proteome heterogeneity during cell differentiation. Genome Biol 19, 161, doi:10.1186/s13059-018-1547-5 (2018).

### Example 2—Surfactant-Assisted One-Pot Sample Preparation for Label-Free Single-Cell Proteomics

### Introduction

Recent advances in nucleic acid amplification-based sequencing technologies allow for comprehensive characterization of genome and transcriptome in single mammalian or tumor cells¹⁻³. Since no protein amplification methods exist for single cell proteome profiling, current single-cell proteomics technologies primarily rely on antibody-based immunoassays (e.g., mass cytometry) for targeted measurements⁴, but they share the limitations of antibody-based approaches⁵. Mass spectrometry (MS)-based proteomics is a promising alternative for quantitative single-cell proteomics because it is antibody-free and has high specificity and ultrahigh multiplexing capability⁶. Sophisticated sample preparation methods are generally used to process standard proteomics samples with large amounts of starting materials (e.g., ≥1000 ug or ≥10 million human cells) for comprehensive proteomic analysis⁷⁻¹⁰. However, they cannot be used to process smaller samples (e.g., low μg or sub-μg levels of starting materials). With this recognition, in the past decade great efforts have been made for effective processing of smaller samples using single-pot sample preparation (e.g., in-StageTip^{11, 12} and SP3^{13, 14}) and immobilized enzyme processing systems (e.g., IMER^{15, 16} and SNaPP¹⁷). Using the in-StageTip device combined with Tip-based sample fractionation, >7000 proteins across 12 immune cell types were reported when ˜15,000 immune cells (˜2 μg) were analyzed¹². The SP3 protocol can allow reproducible quantification of 500-1000 proteins from 100-1000 HeLa cells¹⁴. With improved sample processing as well as recent advances in detection sensitivity, MS-based single-cell proteomics has recently been used for deep proteome profiling of large-size single cells (e.g., oocytes and blastomeres at ˜0.1-100 μg of protein amount per cell)^{13, 18-20}. However, single-cell proteomic analysis of regular-size mammalian cells (typically ˜100 μg per cell) remains highly challenging, primarily due to technical difficulties in effective sampling and processing²¹⁻²³. In recent three years great progress has been made to improve processing recovery from low numbers of cells by either reducing sample processing volume (e.g., nanoPOTS, OAD, and iPAD-1 devices downscaling the processing volume to ˜2-200 nL for label-free global proteomics^{21, 24, 25}) or using excessive amounts of carrier proteins or proteome (e.g., the addition of exogenous BSA as a carrier protein for targeted proteomics^{22, 23} or tandem mass tag (TMT)-labeled 100s of cells as a carrier channel for TMT labeling-based global proteomics²⁶). However, all these approaches have technical drawbacks: nanoPOTS, OAD, and iPAD-1 are not easily adoptable for broad benchtop applications^{21, 24, 25}; exogenous protein carrier is more suitable for targeted proteomics. have; a TMT carrier is added after sample processing, and thus it cannot effectively prevent the surface adsorption losses during initial sample processing²⁶, resulting in low reproducibility with a correlation coefficient of only ˜0.2-0.4 between replicates for ineffectively processed single cells²⁷. Furthermore, due to the inability to fractionate ultrasmall TMT carrier samples, TMT labeling-based global proteomics suffers from ratio compression or distortion caused by coeluting interferences²⁸. Therefore, only three MS-based single-cell proteomics methods are available for reliable label-free analysis of regular-size single mammalian cells, but they need specific devices and/or a skilled person to operate which limit their potential for wide adoptions by research community.

Single-cell proteomics can empower characterization of cell functional heterogeneity and reveal important protein signatures at the single-cell level for rare cell populations, such as cancer stem cells, circulating tumor cells (CTCs), and early metastatic cells. When compared to peripheral blood mononuclear cells (PBMCs), CTCs are rare (normally less than 0.1%). Their seeding efficiency is extremely low but CTCs with stem cell properties can cluster and colonize at relatively high efficiency²⁹⁻³³. CTCs can remain in the blood stream for up to several hours as single cells or tumor clusters, and sometimes they associate with various other cell types (e.g., neutrophils) until they extravasate at a potential site of metastasis^{29, 34-36}. However, there are no available tools for proteomic characterization of CTCs that can elucidate their heterogeneity as well as dynamic alterations upon formation of early micrometastases. Therefore, it still remains uncertain whether metastatic tumor cells undergo an epithelial to mesenchymal transition (EMT) and/or a mesenchymal-to-epithelial transition (MET) at metastatic seeding³⁷⁻⁴⁰.

To alleviate the shortcomings of existing proteomic approaches, the inventors have recently developed a broadly adoptable MS method for quantitative label-free single-cell proteomic analysis. This method capitalizes on surfactant-assisted one-pot (single tube or multi-well plate) processing coupled with MS (termed SOP-MS) for greatly reducing the surface adsorption losses, thus improving detection sensitivity for MS analysis of single cells and mass-limited clinical specimens (FIG. 1). SOP-MS was demonstrated to enable reliable label-free quantification of hundreds of proteins from single cells with standard MS platforms. The inventors applied it to analyze two types of single cells isolated from patient CTC-derived xenografts (PCDX): CTCs propagated in the mouse mammary fat pads with CSC properties (primary tumor cells) and their early micrometastases seeded to the lungs (lung micromets). SOP-MS allows not only for identification of protein signatures from the two different cell types, but also for elucidation of dynamic alterations of metastatic tumor cells upon colonization of the lungs. Interestingly, many of the altered proteins in the lung metastasis are related to the selection pressure of anti-tumor immunity (e.g., neutrophils and innate immunity) for the transition from primary tumor CTCs to the early metastatic cells. These results demonstrate great potential of SOP-MS for broad applications in the biomedical research.

Results

‘All-In-One’ SOP-MS for Maximizing Single-Cell Recovery

The major issue for current MS-based bottom-up single-cell proteomics is substantial surface adsorption losses. Proteins are ‘stickier’ than other biomolecules (e.g., nucleic acids) and need to be digested into peptides for efficient MS analysis which involves multistep sample processing. Both BSA and surfactants are commonly used as additives to minimize surface adsorption for low amounts of proteins and peptides. Unfortunately, the addition of BSA is not suitable for label-free single-cell global proteomics analysis^{22, 23}. Most ionic surfactants (e.g., sodium dodecyl sulfate) are not MS-compatible and require multiple cleanup steps that cause substantial sample loss, especially for small numbers of cells, though they are highly efficient for cell lysis and protein denaturation⁴¹. Nonionic surfactants are known to substantially reduce protein adsorption for hydrophobic surface-based vessels (e.g., single tube or single well) while they have less effects on hydrophilic surfaces (e.g., glass vials), because they have much stronger binding strength than proteins for the hydrophobic surface. They are broadly used to modulate protein aggregation, adsorption loss, stability, and activity in pharmaceutical and biotechnology industries. However, most nonionic surfactants (e.g., octylglucoside) are coeluted with tryptic peptides, which severely affects peptide detection due to ionization suppression⁴².

n-Dodecyl β-D-maltoside (DDM), a classic nonionic surfactant, is an exception. It has been demonstrated to robustly solubilize membrane proteins for effective cell lysis^{43, 44}, and to be highly compatible with MS without requiring surfactant removal and is eluted at a high percentage of organic solvent where it does not impact peptide detection^{43, 44}. Furthermore, DDM is sufficiently thermostable to tolerate the high temperature used for cell lysis and protein denaturation, and can also enhance trypsin and Lys-C enzyme activity⁴². Therefore, the inventors have recently developed a nonionic surfactant DDM-assisted one-pot sample preparation coupled with MS termed SOP-MS that combines all steps into one pot (e.g., single PCR tube or single well from a multi-well PCR plate routinely used for single-cell genomics and transcriptomics) including single-cell collection, multistep single-cell processing, and elimination of all transfer steps with direct sample loading for LC-MS analysis (FIGS. 1a-1c). This ‘all-in-one’ SOP-MS method presumably maximizes single-cell recovery for quantitative single-cell proteomics by greatly reducing possible surface adsorption losses.

To reliably evaluate the performance of SOP-MS, label-free MS was used for proteomic analysis of one cell at a time and protein identification is solely based on the actual MS/MS spectra from the analyzed cell, which is the cornerstone of MS-based proteomics. Furthermore, once it works for label-free MS analysis, SOP-MS can be widely used for other types of MS analysis of single cells. A commonly accessible Q Exactive Plus MS platform was used for the development of SOP-MS and its application demonstration.

Evaluation of SOP-MS Performance Using Peptides and Low-Input Human Cell Lysates

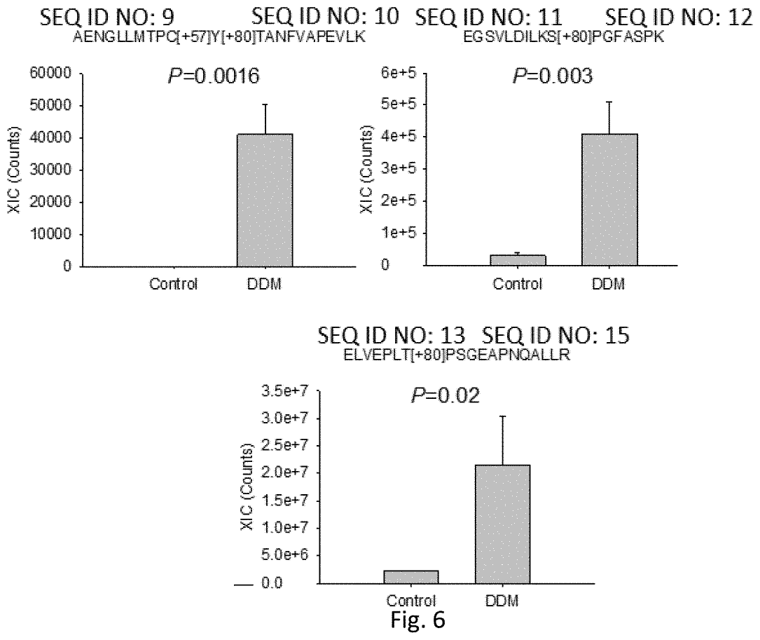

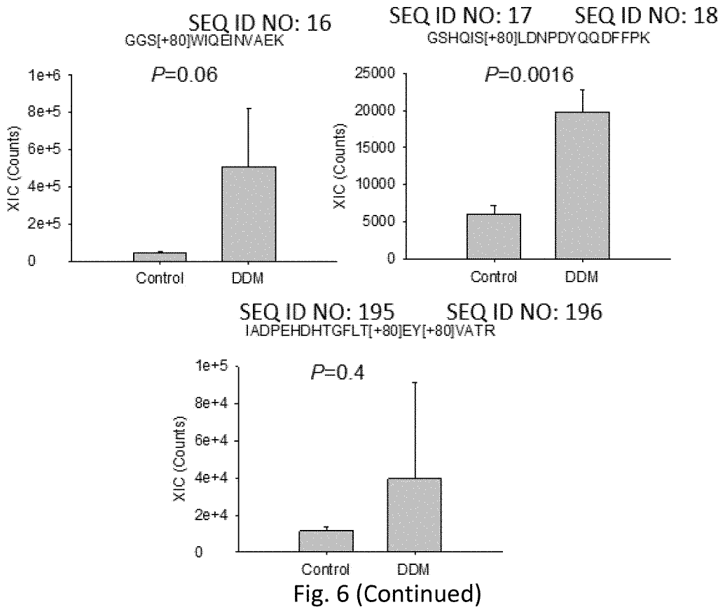

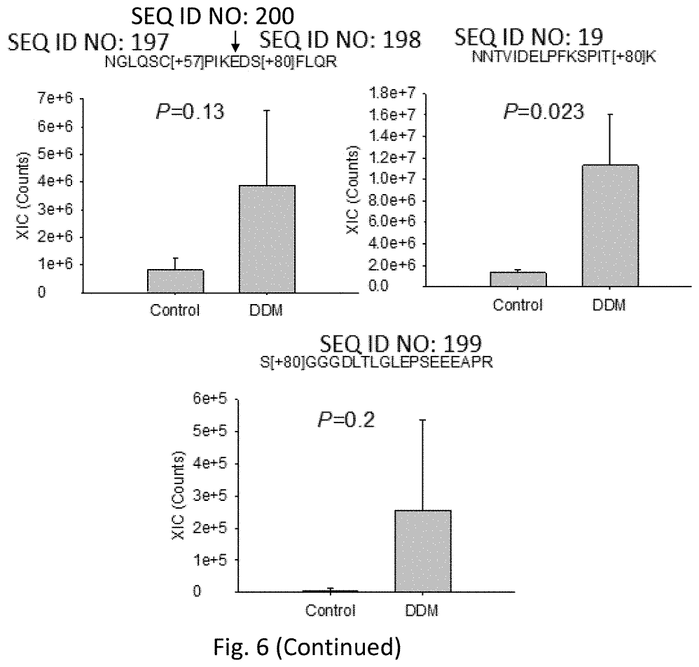

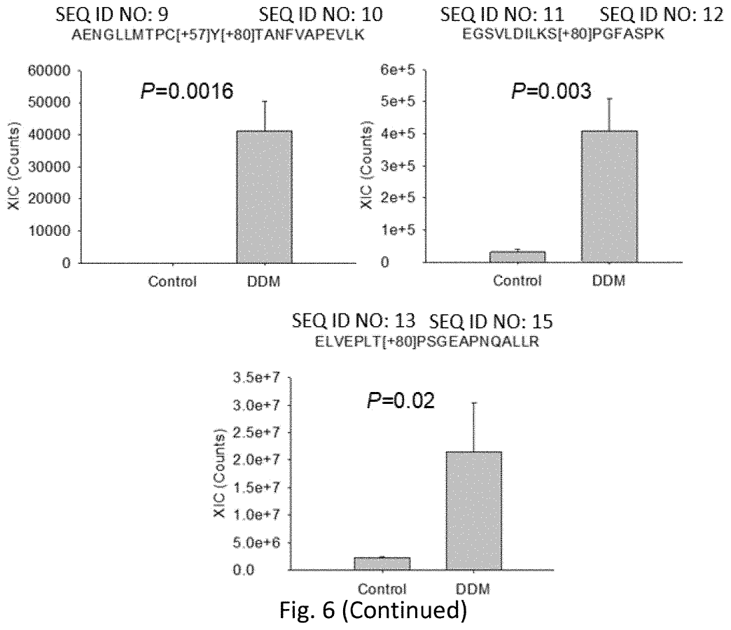

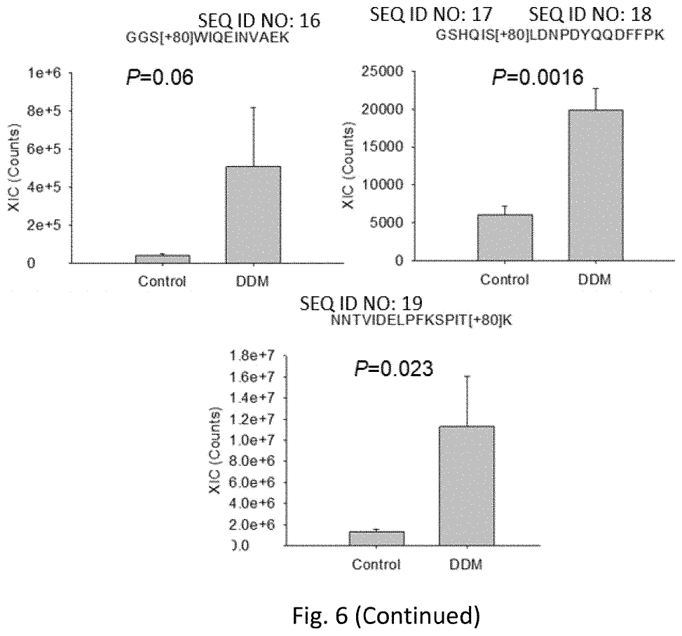

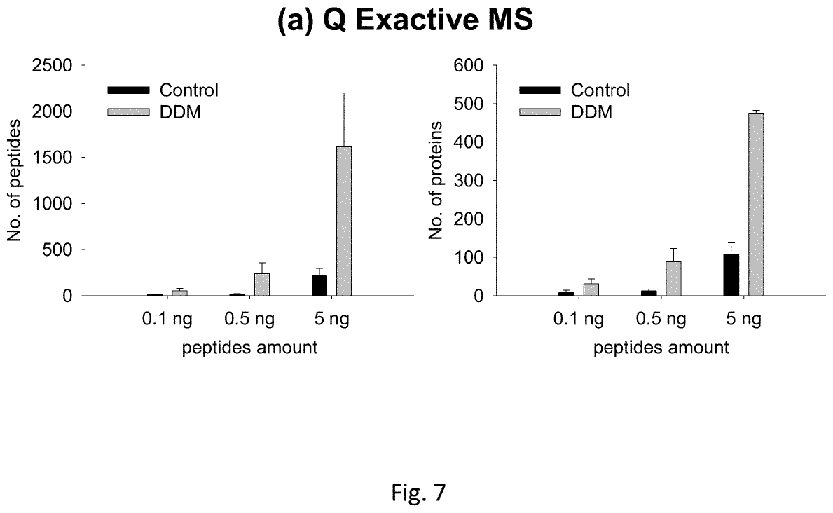

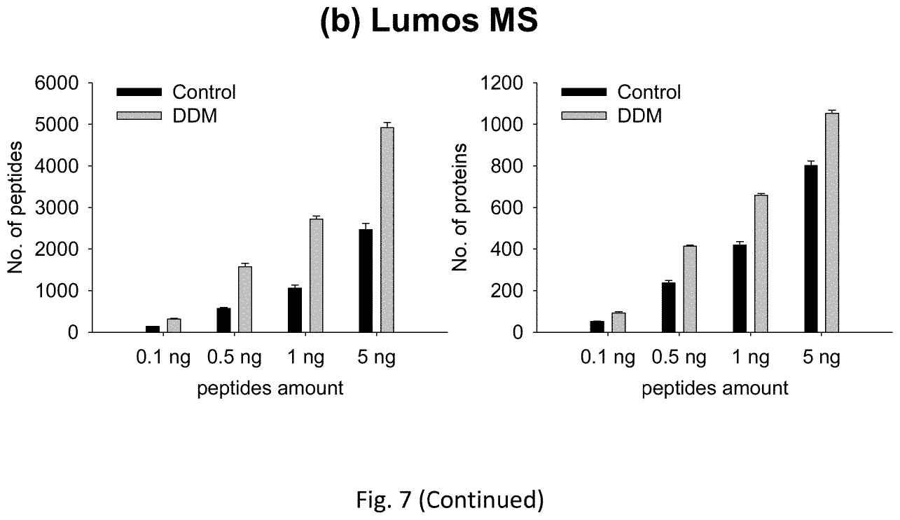

To achieve precise proteome quantification of single cells the inventors systematically evaluated sample recovery and processing reproducibility using more uniform low-input (small) samples (i.e., cell lysates or protein digests) with and without DDM in single PCR tubes. Selected reaction monitoring (SRM)-based targeted proteomics was used to optimize DDM concentrations from 0.005% to 0.1% due to its demonstrated higher reproducibility and quantitation accuracy when compared to global proteomics. Heavy isotope-labeled EGFR pathway peptide standards at a fixed concentration were measured at different DDM concentrations. The best SRM signals for most EGFR pathway peptides was achieved with 0.01-0.02% DDM, where higher DDM concentration can saturate the LC column and thus greatly degrade chromatographic performance. For simple peptide standard mixtures, 0.015% DDM was demonstrated for enabling to increase SRM signals by 3-35-fold with an average of ˜20-fold improvement (FIG. 6). The inventors further evaluated DDM-assisted performance for single-cell level mass input of tryptic peptide mixture (i.e., 0.2 ng of acute myeloid leukemia (AML) cell lysate digests). With the addition of 0.015% DDM, the number of identified peptides (proteins) greatly increased from 63 (53) to 891(342) with ˜20-fold enhancement in MS signal and significant difference was observed between without and with DDM (FIGS. 1d-1e). Additional experiments from different groups have recently been conducted to further confirm the efficiency of DDM for low mass input of tryptic peptide mixture from lung cancer PC9 cell lysate digests (FIG. 7). All these results clearly demonstrated that the feasibility of SOP-MS for analysis of sub-ng quantities of cell lysate digests (<10 mammalian cells).

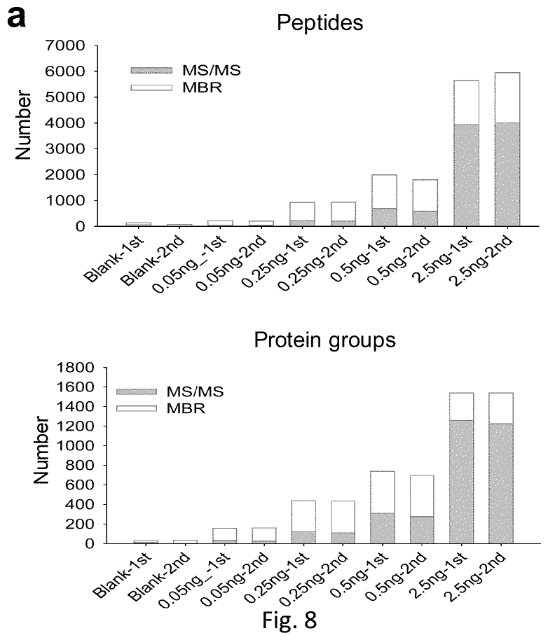

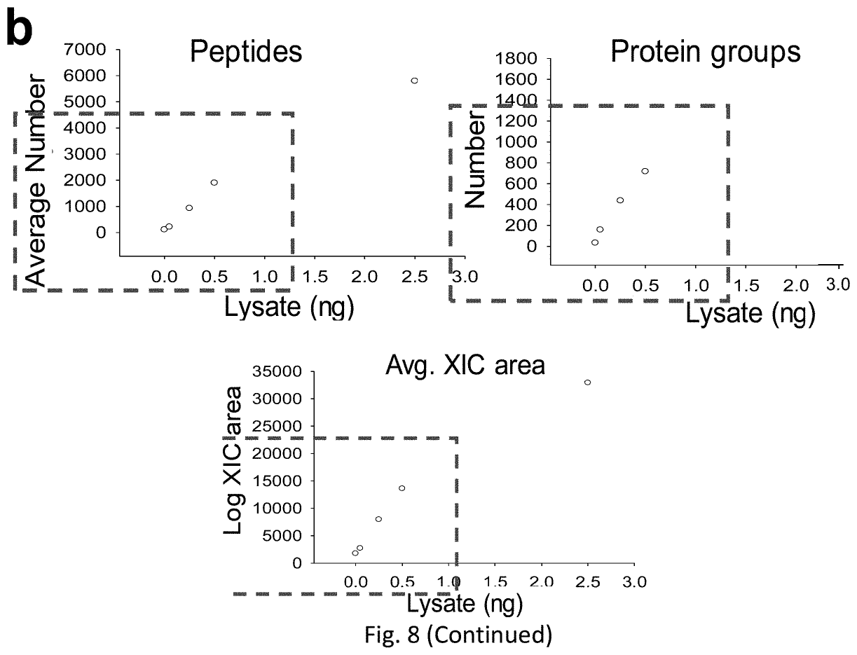

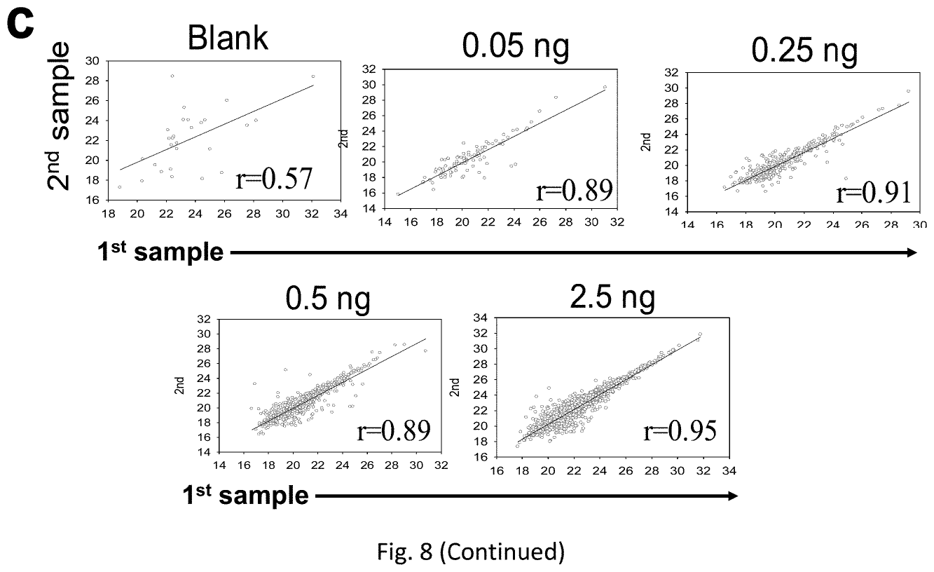

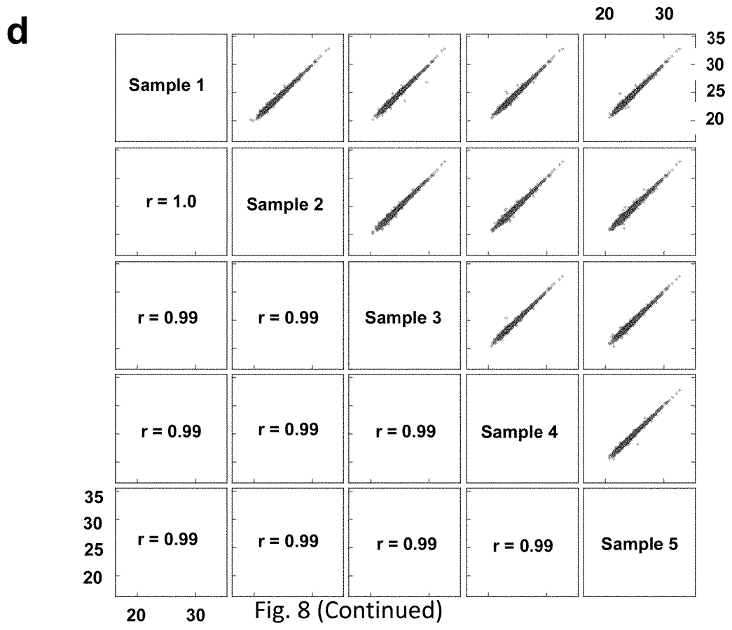

The inventors next evaluated the performance of SOP-MS by serial dilution of uniform human breast cancer MCF7 cell lysates at 0.05-2.5 ng (close to 0.5-25 cells in protein mass) in the low-bind 96-well PCR plate (Methods). For 0, 0.05, 0.25, 0.5 and 2.5 ng of proteins, after trypsin digestion the average number of identified peptides (protein groups) was 38(7), 47 (31), 214 (116), 639 (293) and 3971 (1241), respectively. With the use of a MaxQuant MBR (match-between-run) function, the number of identified peptides (protein groups) consequently increased to 110 (33), 217 (156), 928 (437), 1897 (717) and 5792 (1539), respectively (FIG. 8a). To evaluate the quantitation accuracy of SOP-MS, the inventors have built three types of response curves, the number of unique peptides, the number of protein groups, and the log 2 extracted ion chromatogram (XIC) area as a function of low sample inputs (FIG. 8b). All the response curves have good linearity with a correlation coefficient (R²) of ˜0.99 from 0 to 0.5 ng, reflecting accurate quantification with a linear dynamic range for analysis of small number of cell equivalents by SOP-MS. Furthermore, SOP displayed high reproducibility with an average of Pearson correlation coefficient of ˜0.90 for 0.05-0.5 ng (close to 0.5 and 5 human cells) (FIG. 8c) and ≥0.99 between any two out of 5 replicates for 5 ng (FIG. 8d). All the results have demonstrated that the ‘all-in-one’ SOP-MS enables for reproducible quantitative analysis of low mass inputs of cell lysates (close to one cell or low numbers of cells in protein mass).

SOP-MS for Label-Free Proteomic Analysis of Small Tissue Sections

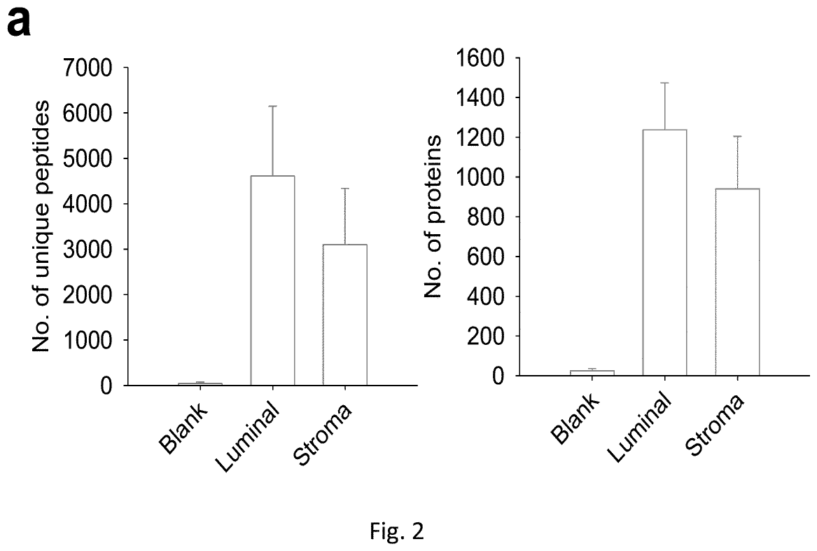

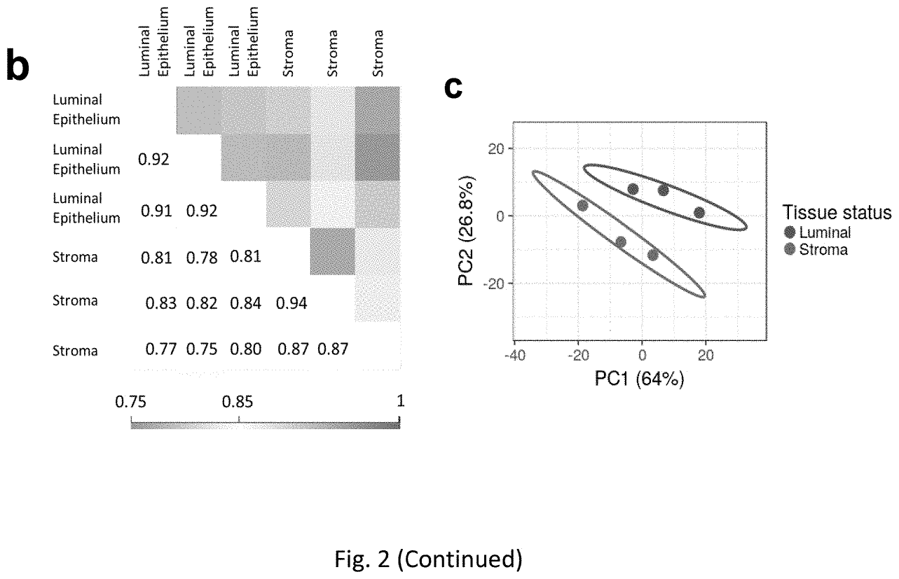

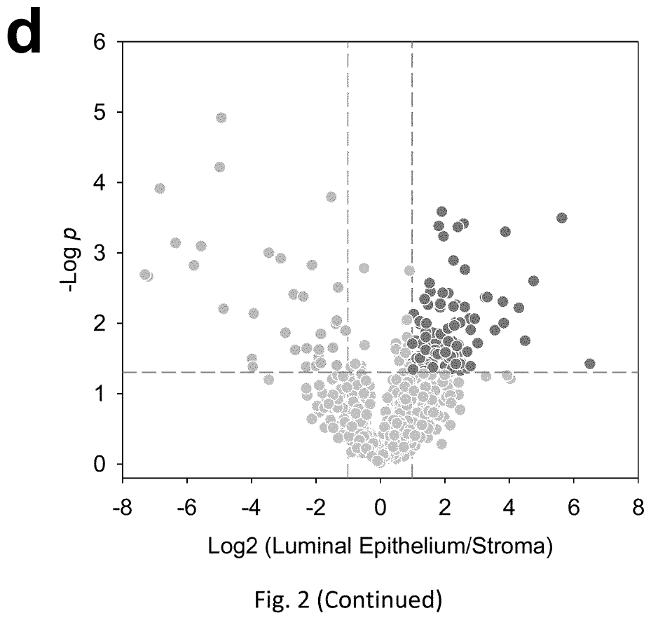

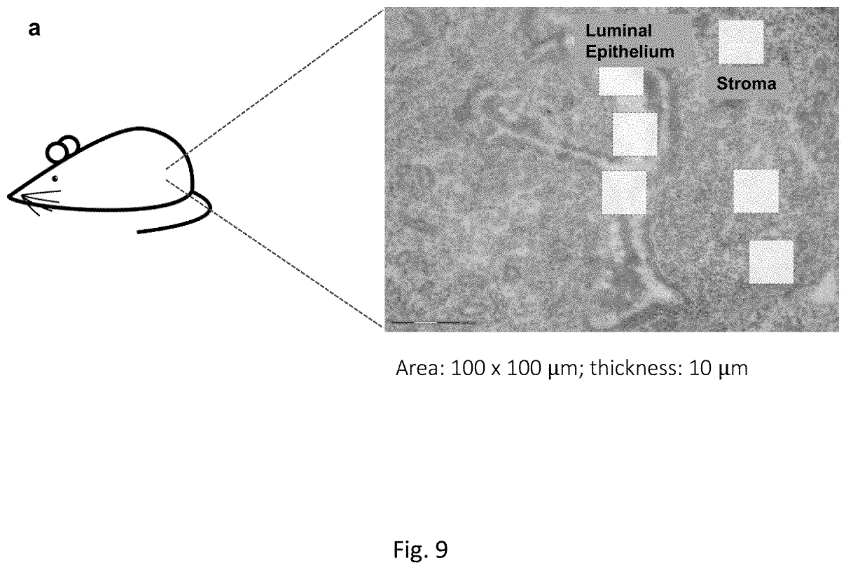

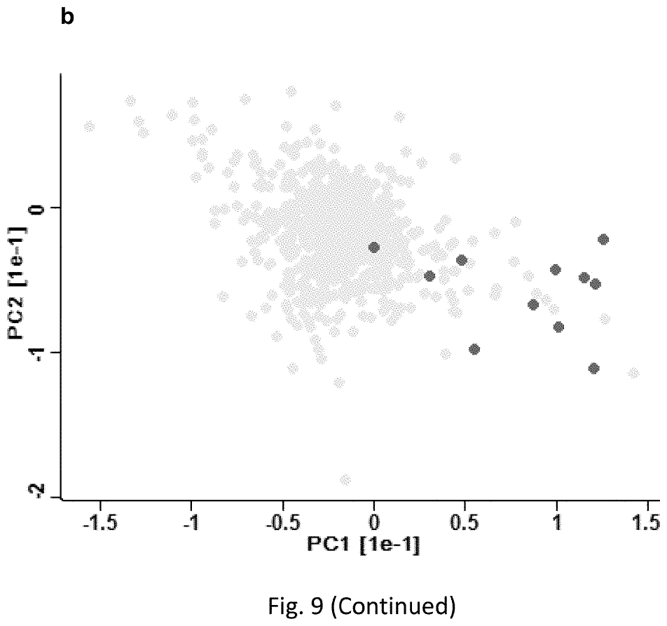

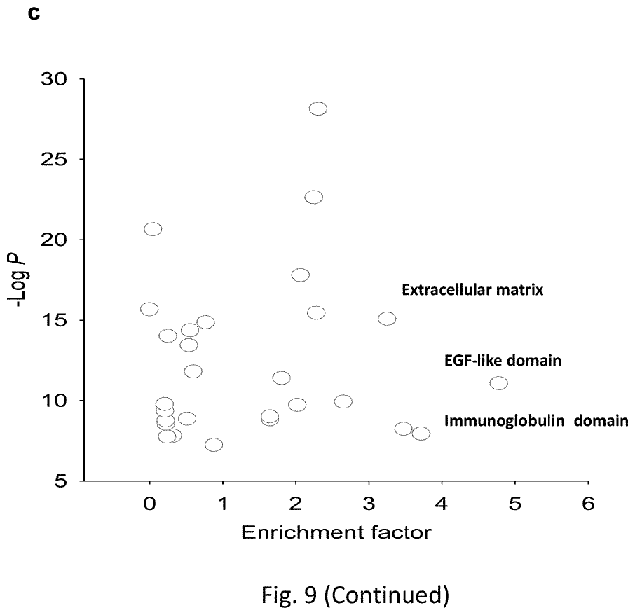

With its demonstrated improvement in analyzing low-input samples, the inventors next evaluated whether SOP-MS can be used for label-free, global proteomics analysis of small numbers of cells derived from mouse uterine tissues (FIG. 1). Two distinct regions of luminal epithelium and stroma were dissected by laser capture microdissection (LCM) in three replicates, each with a tissue spot size of 100 μm in diameter and 10 μm in thickness (close to ˜20 cells based on recent study of small tissue sections⁴⁵) (FIG. 9a). These tissues were analyzed by SOP-MS for label-free proteome profiling (FIG. 1). A total of ˜7,600 unique peptides (˜1,340 protein groups) were identified from luminal epithelium, and ˜5,200 unique peptides (˜1,100 protein groups) from stroma (FIG. 2a). Pairwise analysis of any two tissue samples showed Pearson correlation coefficients ranging from 0.75 to 0.94 (FIG. 2b). As expected, the correlation from the same sub-region replicates is higher than that from different sub-region replicates (FIG. 2b). This further confirmed high reproducibility of SOP-MS for processing small numbers of cells.

To evaluate whether the identified proteins can be used to specify tissue regions, the inventors performed principal component analysis (PCA). The luminal epithelium and stroma regions were clearly segregated based on the protein expression alone with the three biological replicates from the same regions being clustered together (FIG. 2c). To identify protein features distinguishing the two regions, analysis of variance (ANOVA) was performed with a volcano plot of differentially expressed proteins (FIG. 2d), revealing ˜15% of quantified proteins (˜160 proteins) to be significantly different with p<0.05. Among the differential proteins, some of them are expected to be cell-type specific: cell junctional proteins (e.g., catenins and filamin B) and hydrolases (e.g., calpain 1 and neprilysin) for luminal epithelial cells, and extracellular matrix proteins (e.g., decorin, collagen, laminin and fibronectin) for stromal cells (FIGS. 9b-9c). Thus, SOP-MS was demonstrated to enable precise deep proteome profiling of small numbers of cells from LCM-dissected tissues.

SOP-MS for Label-Free Quantitative Single-Cell Proteomics

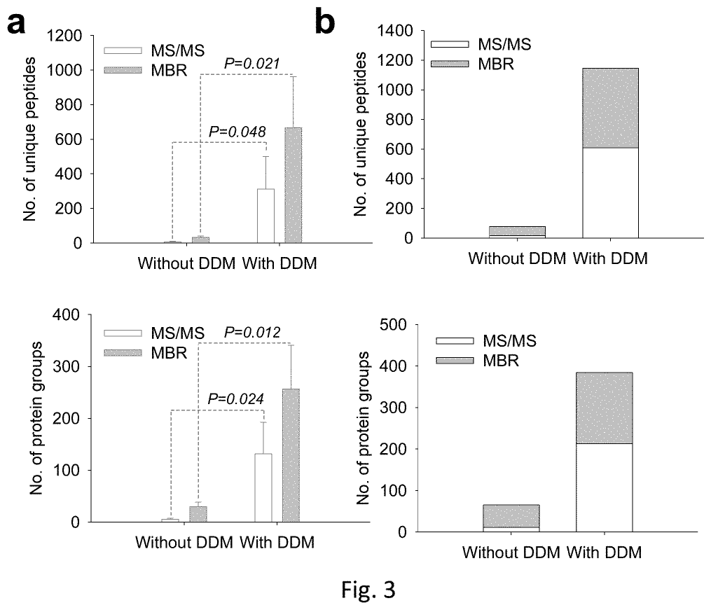

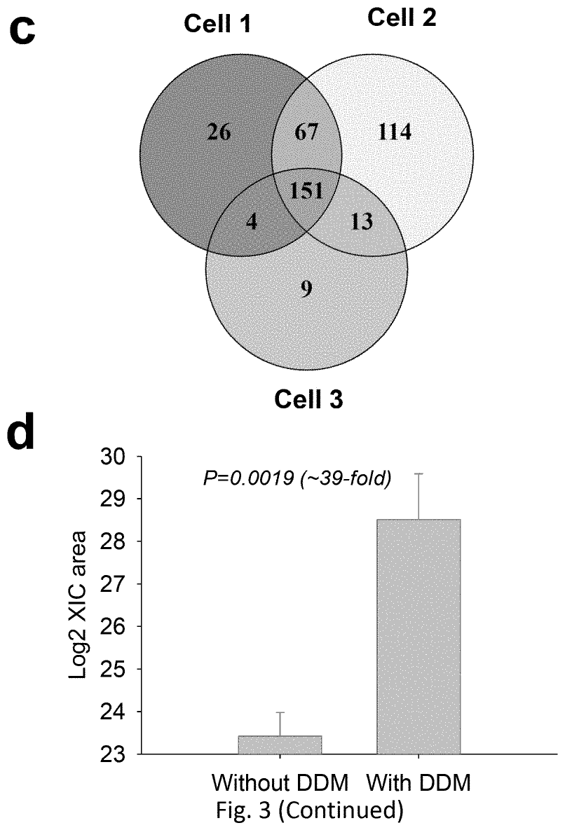

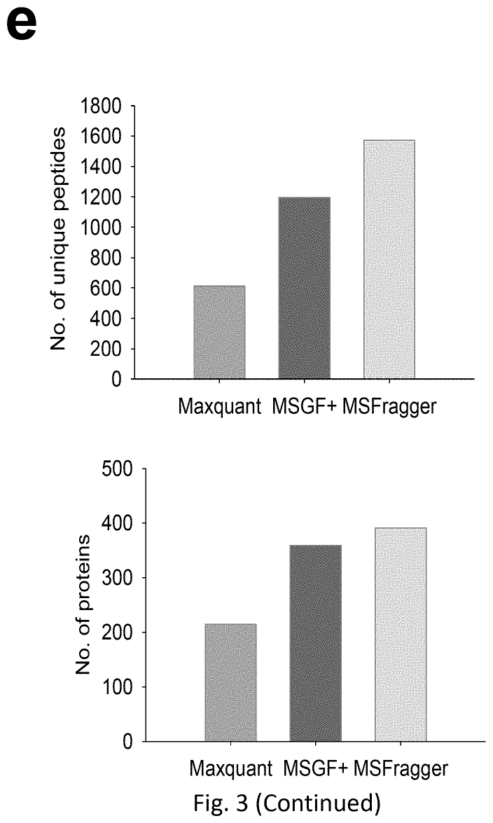

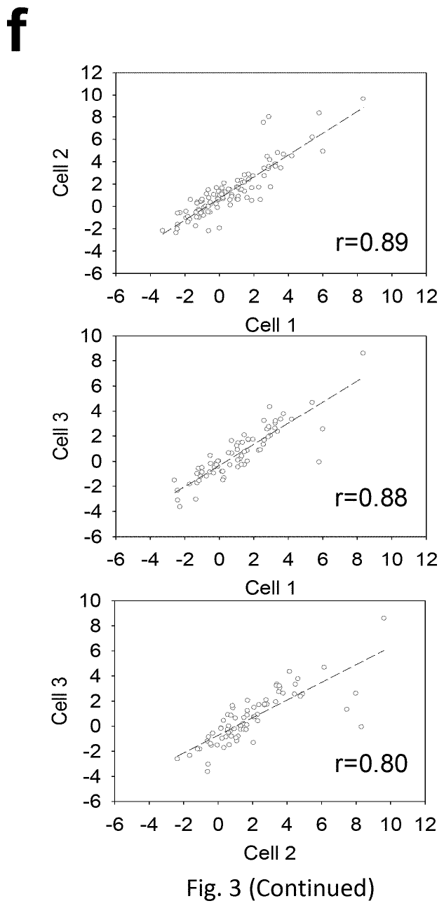

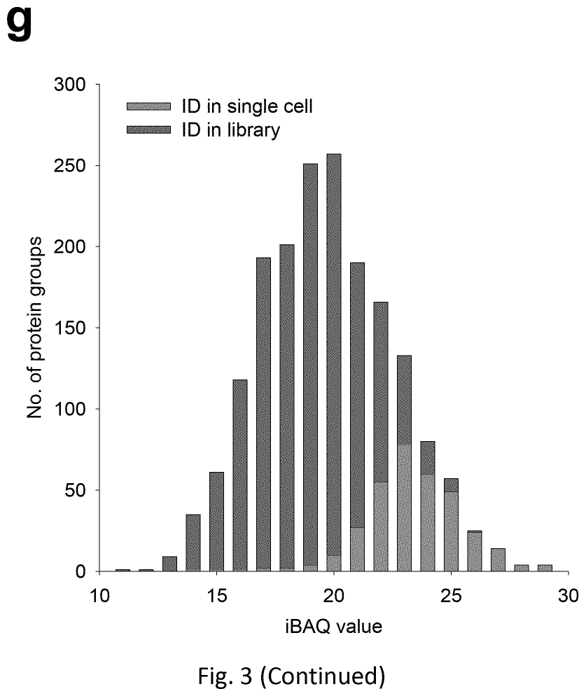

With the demonstrated performance for small numbers of cells, the inventors evaluated whether SOP-MS can be used for proteomic analysis of single mammalian cells. Single cells were sorted directly into single low-bind PCR tubes (one cell per tube) by fluorescence-activated cell sorting (FACS). Single MCF10A cells were processed without and with 0.015% DDM (three biological replicates per condition) in parallel by SOP followed by LC-MS analysis (FIG. 1). With the DDM additive the average number of unique peptides identified from biological triplicates was 313, resulting in identification of 131 protein groups with the MS/MS spectra alone (i.e., without MBR) (FIG. 3a). By contrast, without DDM the average number of unique peptides was only 6, corresponding to 5 protein groups. Furthermore, significant difference was observed between without and with DDM (FIG. 3a). This result strongly suggests that without the DDM additive the ‘all-in-one’ one-pot method cannot effectively process single cells for proteomic analysis, consistent with our observation for cell lysate digests and peptide standards.

To increase the number of identified unique peptides (protein groups), other commonly used proteomic algorithms were used to reanalyze the single-cell data. With the use of MBR function in MaxQuant, the average protein identifications were increased to 229, and a total of 384 protein groups were identified across three biological replicates for single MCF10A cells (FIG. 3b). 151 protein groups were commonly identified for all 3 single MCF10A cells, and an average of ˜53% protein groups overlapped between any two single MCF10A cells, suggesting cell-to-cell variability (FIG. 3c). An average of ˜39-fold enhancement in MS signal was observed with significant difference between samples without and with DDM (FIG. 3d), which further confirmed the importance of using DDM additive for single-cell processing. When compared to MaxQuant search with identification of a total 215 protein groups by the MS/MS spectra alone across three MCF10A biological replicates, other two common software tools MSGF+ and MSFragger were evaluated with enabling identification of 359 protein groups for MSGF+ and 391 protein groups for MSFragger (FIG. 3e). These results have further confirmed that SOP-MS enables confident detection of hundreds of proteins from single human cells. Among the three software tools, MaxQaunt is the most commonly used tool for label-free quantification. Unless otherwise mentioned, MaxQuant was used for quantitative analysis of all the single-cell proteomics data. The inventors next evaluated the reproducibility of SOP-MS for quantitative single-cell proteomic analysis. High reproducibility was demonstrated with Pearson correlation between any two single cells of 0.80-0.89 for single MCF10A cells (FIG. 3f). To evaluate the measurement reliability by SOP-MS, the inventors compared the abundance distribution of proteins identified in single cells with that from 10 ng MCF10A cell lysate digests. As expected, most proteins identified in single cells were highly abundant and above the median abundance of the 10 ng MCF10A cell lysate digests (FIG. 3g). Therefore, SOP-MS enables precise, quantitative, label-free single-cell proteomics.

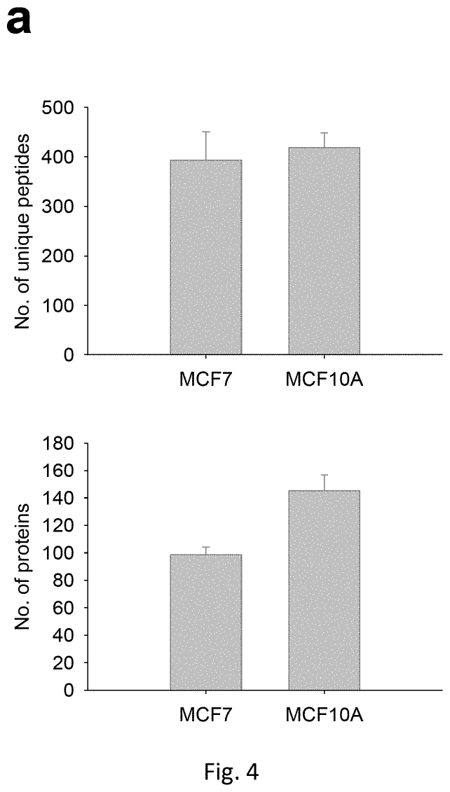

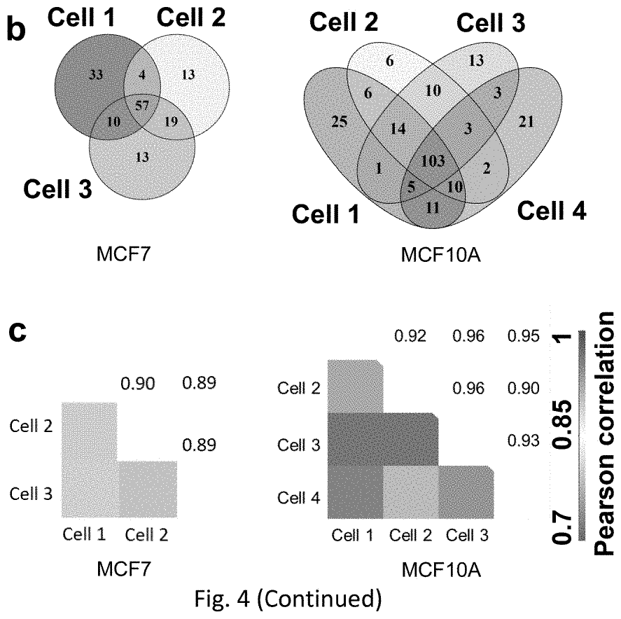

To validate SOP-MS for single-cell proteomics analysis the inventors performed an independent experiment for 4 single cells sorted by FACS from newly cultured MCF10A cells. An average of 146 protein groups were identified with the MS/MS spectra (FIG. 4a) and 103 protein groups were commonly identified for all the 4 single MCF10A cells (FIG. 4b). An average of ˜64% protein groups overlapped between any two singe cells, suggesting lower cell-to-cell variability when compared to the above 3 single MCF10A cells (FIG. 3c). This was further confirmed by the higher median correlation coefficient (˜0.94) (FIG. 4c) than that from the above 3 single MCF10A cells (FIG. 3f). In addition, SOP-MS was used for analysis of different types of cells, 3 single MCF7 cancer cells with half of sample injection (i.e., ˜0.5 single cells for MS analysis) to mimic other small-size single mammalian cells. An average of 98 protein groups were identified from half of single MCF7 cells with a correlation coefficient of 0.9 (FIG. 4). All these results further confirmed high reproducibility of SOP-MS for reliable label-free quantification of 100s of proteins from single mammalian cells.

Application of SOP-MS to Single Cells Derived from a PCDX Model

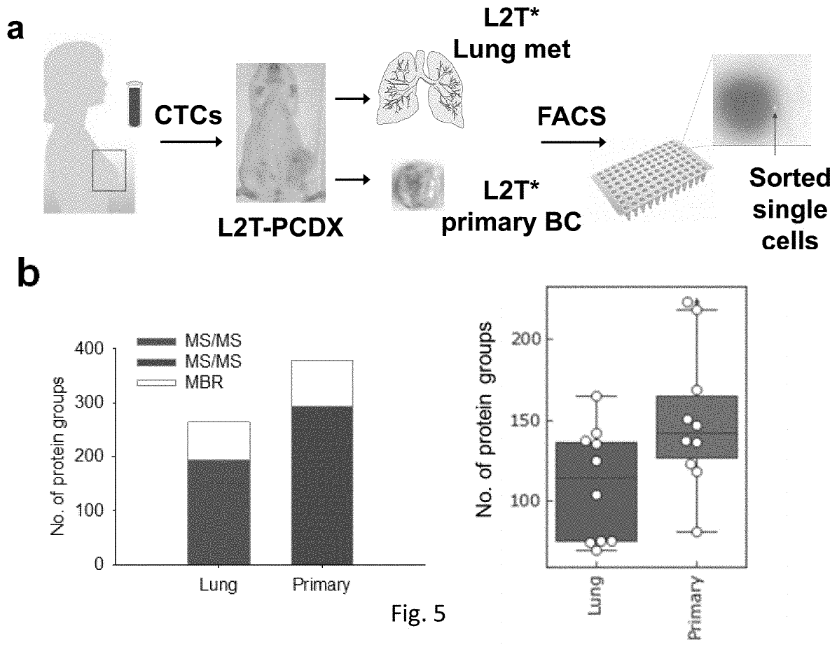

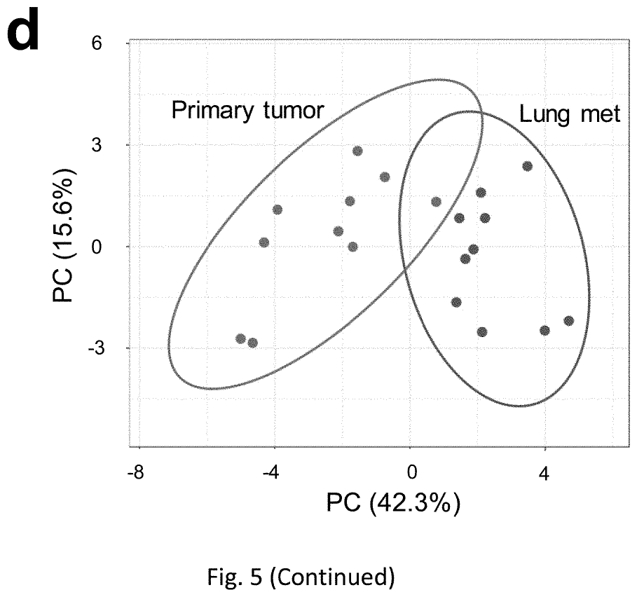

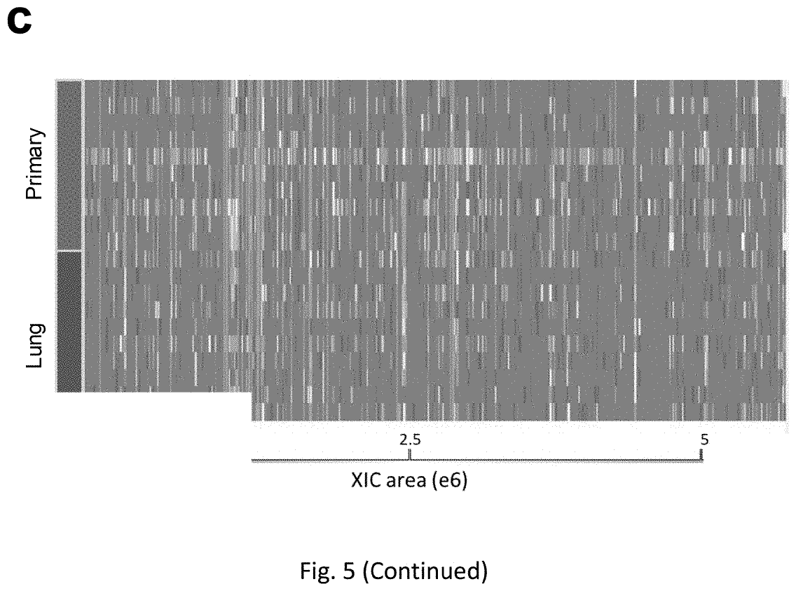

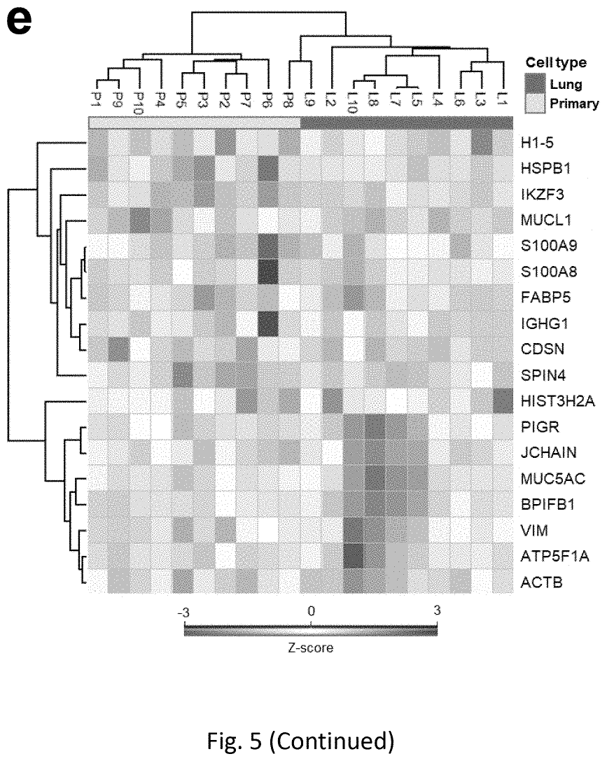

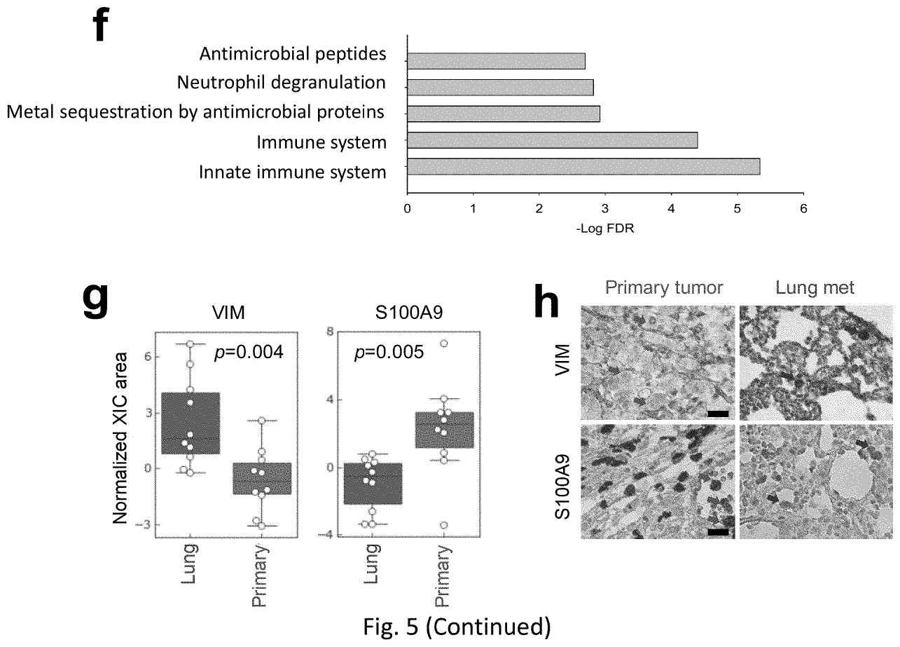

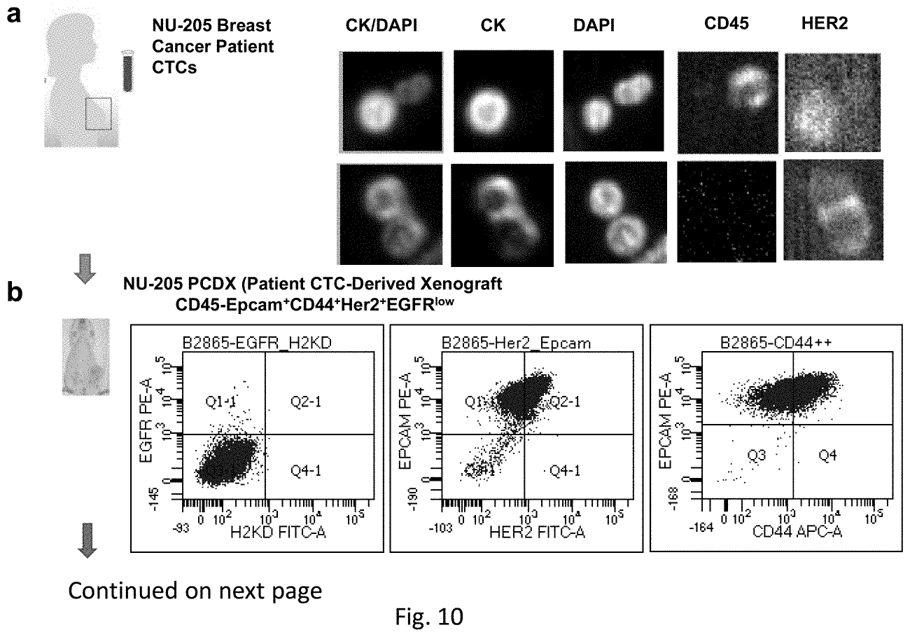

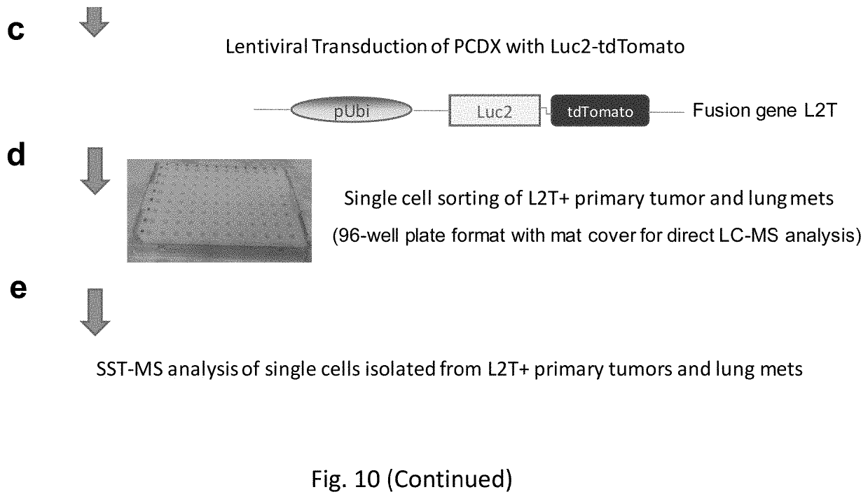

To demonstrate the potential applications of SOP-MS to cancer research as well as to evaluate whether identification of hundreds of relatively abundant proteins can provide meaningful biological insights into cellular heterogeneity, the inventors applied SOP-MS for single-cell proteomic analysis of primary tumors and early lung metastases in a PCDX mouse model generated from patient CTCs (FIG. 10). After dissociation of luciferase 2-tdTomato (L2T)-labeled PCDX tissues, single L2T⁺ tumor cells were sorted by FACS into 96-well PCR plates (one cell per well) with ten from propagated CTCs (primary) and ten from metastases (lung) (FIG. 5a). With the MaxQuant MBR function, a total of 265 proteins were identified across all 10 single lung metastatic cells with the range of 69-163 protein groups for each single cells, and a total of 379 proteins identified across all 10 single primary tumor cells with the range of 81-223 protein groups for each single cells (FIG. 5b). The total XIC peak area for each protein group across the 20 single cells was presented as a heatmap for an overview of protein group detection (FIG. 5c). The higher number of protein identification from single primary tumor cells is consistent with their relatively larger size when compared to lung cells (breast tumor cells: ˜12 μm in diameter⁴⁶ and lung cells: ˜8 μm in diameter⁴⁷), reflecting the reliability of SOP-MS for single-cell proteomic analysis.

Unsupervised PCA analysis has shown distinct clustering of proteins from the primary CTCs versus the lung metastases (FIG. 5d), with significant abundance changes for 18 proteins between the two cell types (FIG. 5e). Cellular heterogeneity within the same cell type and between the two different cell types was clearly observed based on protein abundance achieved by label-free quantification (FIG. 5d). Based on pathway analysis, many of these proteins differentially expressed in the early metastases are annotated as immune related proteins (e.g., S100 calcium-binding family proteins A8 and A9, IGHG1, PIGR and BPIFB1) (FIG. 5f). This may infer tumor cell alterations enabling immune evasion in response to dynamic selection pressure of anti-tumor immunity from the transition of primary tumor cells to early metastasis. With literature mining, many proteins showing reduced abundance in the lung metastases are associated with epithelial cell differentiation (e.g., CDSN) or epithelial cancers (e.g., S100A family proteins⁴⁸ and MUCL1 small breast epithelial mucin⁴⁹), consistent with the cell-type plasticity between primary tumor and early metastasis. Notably, in the lung metastases the EMT markers, vimentin (VIM), MU5AC⁵⁰ and PIGR⁵¹, displayed significant upregulation (FIG. 5e), suggesting the occurrence of EMT in early micrometastatic cells. Meanwhile, downregulated two chaperone proteins (HSPB1^{52, 53} and FABP5⁵⁴) reported to promote EMT, may infer altered adaptation states in the lung metastatic cells (FIG. 5e).

To further validate label-free MS quantification, two representative proteins, VIM and S100A9, were selected with median expression upregulated and downregulated by 4.7 and 8.6 in the lung metastatic cells, respectively (FIG. 5g). The two proteins were measured with immunohistochemistry (IHC) staining of the primary tumor and lung tissue sections from the original PCDX model used for sorting single L2T⁺ tumor cells. Results from IHC staining are in agreement with the data from label-free MS quantification (FIG. 5h), which confirmed reliable single-cell proteomic quantification with SOP-MS.

Discussion

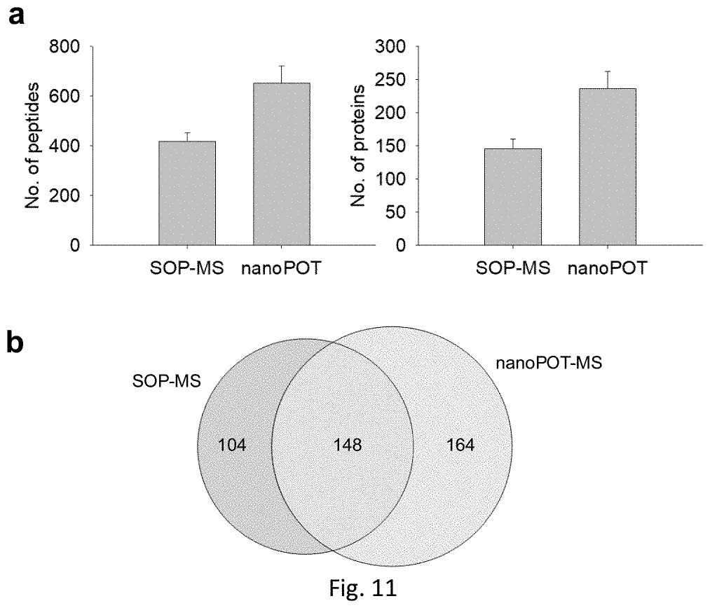

SOP-MS is a convenient robust method for label-free single-cell proteomics, where single cells are processed in either low-bind single tubes or multi-well plates which are routinely used for single-cell genomics and transcriptomics. The performance of SOP-MS (e.g., sensitivity, reproducibility, and quantitation accuracy) was demonstrated by label-free MS analysis of low mass inputs from serial dilution of uniform MCF7 cell lysates, LCM-dissected small tissue sections, and FACS-sorted single cells. Based on the actual MS/MS spectra for reliable protein identification (without using the MBR function) which is the cornerstone of MS-based proteomics, SOP-MS can identify ˜146 protein groups from single human cells, higher than ˜128 for iPAD1-M5²⁴ and 51 for OAD-M5²⁵ and ˜1.4-2.5-fold lower than ˜211-362 for nanoPOTS-M5⁵⁵⁻⁵⁷ (Table 1), and ˜1200 proteins from small tissue sections (close to ˜20 cells). Comparative analysis of single MCF10A cells using both SOP-MS and nanoPOTS-MS has shown that the number of protein groups from SOP-MS is ˜1.6-fold lower than that from nanoPOTS-MS and ˜60% of protein groups from SOP-MS overlapped with the protein groups from nanoPOTS-MS (FIG. 11 and Table 1). Most importantly, unlike all currently available label-free single-cell proteomics methods that need specific devices and are difficult to access by research community, SOP-MS has advantages in terms of high compatibility with cell sorting or tissue collection systems and LC-MS analysis using single tubes or multi-well plates (FIGS. 1a-1c), and high flexible scalability shifting from single tube to multi-well plate for one-pot sample preparation. Thus, SOP-MS is easy to be widely adopted by research community for broad applications. Furthermore, automation of the whole ‘all-in-one’ sample preparation workflow can be readily achieved for high sample throughput by using commercially available liquid handlers for precisely dispensing μL or sub-μL reagent solution. Therefore, SOP-MS represents a breakthrough in technology for label-free MS-based single-cell proteomics.

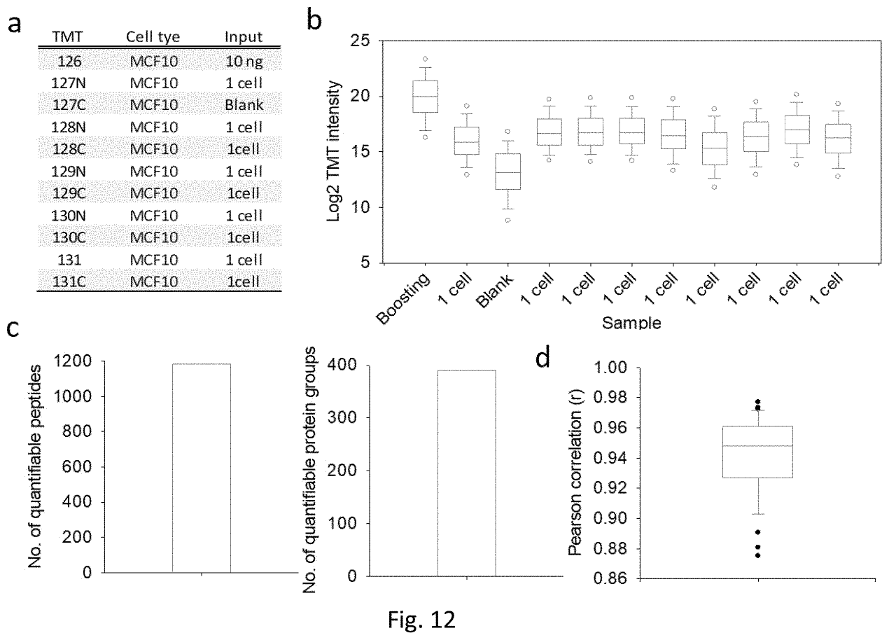

With its demonstration for label-free MS analysis, SOP-MS can be equally used for other types of single-cell proteomic analysis (e.g., targeted proteomics and TMT-based MS analysis). It can also be used for analysis of other ultrasmall precious clinical specimens (e.g., rare CTCs and tissues from fine needle aspiration biopsy). The inventors have initially evaluated integration of our recently developed TMT-based BASIL strategy⁵⁸ into SOP-MS for multiplexed analysis of 9 single MCF10A cells. A median correlation coefficient of ˜0.95 was achieved (FIG. 12d) primarily due to high recovery and reproducibility of SOP-MS.

Future developments will focus on improvements in detection sensitivity and sample throughput for rapid deep proteome profiling of single mammalian cells. Enhancing detection sensitivity could be achieved by effective integration of ultralow-flow LC or capillary electrophoresis (CE) and a high-efficiency ion source/ion transmission interface with the most advanced MS platform. Further improvement can be gained by further reducing sample loss (e.g., systematic evaluation of different types of MS-friendly surfactants) and increasing reaction kinetics through reducing processing volume from 10-15 μL down to 1-2 μL with automated small-volume liquid handling (e.g., automated MANTIS liquid handler). All these improvements in detection sensitivity will lead to greatly increase the measurement reliability (e.g., more high-quality MS/MS spectra) as well as the number of identified peptides/protein groups. Sample throughput could be increased by using ultrafast high-resolution ion mobility-based gas-phase separation (e.g., SLIM⁵⁹) to replace current slow liquid-phase (LC or CE) separation, and effective integration of liquid- and gas-phase separations (e.g., SLIM⁵⁹ or FAIMS⁶⁰) for greatly reducing separation time but without trading off separation resolution. Alternatively, sample multiplexing with isobaric barcoding and implementation of a multiple LC column system can also be considered to increase sample throughput. All these improvements could lead to a more powerful SOP-MS platform and will certainly close the gap between single-cell proteomics and single-cell transcriptomics or genomics.

When compared to proteomic analysis of bulk cells that only provides the averaged expression signal, single-cell proteomics can provide a clean signal for single cells of interest without signal contribution from other types of cells, allowing to uncover new biological discoveries. When applied for analysis of single cells derived from a clinically relevant PCDX model, SOP-MS can reveal distinct protein signatures between primary and metastatic tumors as well as cellular heterogeneity within the same cell type. Proteins with altered expression levels are involved in tumor immunity (e.g., S100A family members⁶¹), epithelial cell differentiation (e.g., CDSN), and EMT (vimentin^{38, 62}), suggesting possible selective pressure for immune evasion and cell state plasticity. The data provide a clear path for future mechanistic studies of cancer metastasis with the potential to guide targeted cancer therapy. SOP-MS analysis of single cells is under way to reveal robust protein signatures related to physiological and pathological states at the single-cell resolution. Furthermore, with its demonstration for analysis of CTC-derived single cells, SOP-MS can be equally applied to clinically important patient CTCs that link disseminated and primary tumors. Thus, it has great potential for liquid biopsy-guided diagnostic and prognostic applications as well as for rational therapeutic intervention.

In summary, the inventors report an easily implementable SOP-MS method that capitalizes on using surfactant-assisted one-pot sample preparation to reduce the surface adsorption losses for label-free single-cell proteomics. Label-free quantitative proteome profiling of single cells can be achieved with easily accessible sample preparation devices (single tubes or multi-well plates) and standard LC-MS platforms. With its convenient features, SOP-MS can be readily implemented in any MS laboratory for single-cell proteomic analysis. The application of SOP-MS to single cells derived from a PCDX model demonstrated its power for precise characterization of cellular heterogeneity and discovery of distinct protein signatures related to breast cancer metastasis. With improvements in detection sensitivity and sample throughput as well as automation for high sample throughput, the inventors believe that SOP-MS has great potential to close the gap between single-cell proteomics and single-cell transcriptomics, and could open an avenue for single-cell proteomics with broad applicability in the biological and biomedical research.

TABLE 1
Overview of current MS-based single-cell proteomics for label-
free proteome profiling of regular-size single human cells.

| Single-cell | | | | | | |
|---|---|---|---|---|---|---|
| processing method | nanoPOTS | | | iPAD-1 | OAD | SOP |
| LC-MS setup | LC flow rate: | LC flow rate: | LC flow rate: | LC flow rate: | LC flow rate: | LC flow rate: |
| | 60 nL/min | 20 nL/min | 60 nL/min | 40 nL/min | 200 nL/min | 100 nL/min |
| | MS: Orbitrap | MS: Orbitrap | MS: Orbitrap | MS: Orbitrap | MS: Orbitrap | MS: Q |
| | Fusion Lumos | Fusion Lumos | Fusion Lumos | Fusion Lumos | MS | Exactive plus |
| | | and Eclipse | | | | |
| Single cells | HeLa | HeLa | MCF10A | HeLa | HeLa | MCF10A |
| MS/MS only | 211 | 362 | 236 | 128 | 51 | 146 |
| (The number of | | | | | | |
| identified | | | | | | |
| protein groups) | | | | | | |
| Device | Chip-based microfluidic nanodroplet | | | In small i.d. | Nanoliter- | PCR tube |
| | | | | (22 μm) | scale oil-air- | or multi- |
| | | | | capillary | droplet chip | well plate |
| Reference | Angew Chem, | Anal Chem, | Recent | Anal Chem, | Anal Chem, | Current |
| | 130 (2018), | 92 (2020), | experiments | 90 (2018), | 90 (2018), | work (4 |
| | 12550-12554 | 2665-2671 | (4 replicates) | 14003-14010 | 5430-5438 | replicates) |

Methods

Human Sample Collection and Animal Studies

The human blood analyses for breast cancer patients were approved by the Institutional Review Boards at Northwestern University and complied with NIH guidelines for human subject studies. Animal procedures and experimental procedures have been performed under approval by Northwestern University Animal Care and Use Committee (ACUC) and complied with the NIH Guidelines for the Care and Use of Laboratory Animals. 8-10 weeks old female NSG mice were used for implantation of human breast cancer PCDX models and kept in specific pathogen-free facilities in the Animal Resources Center at Northwestern University. Breast tumors were harvested after 2-3 months and confirmed as a human PCDX with positive expression of human epithelial markers EpCAM, HER2, and CD44 as well as negative expression of mouse H-2Kd.

Reagents

n-Dodecyl β-D-maltoside (DDM), dithiothreitol (DTT), iodoacetamide (IAA), ammonium bicarbonate, acetonitrile, and formic acid were obtained from Sigma-Aldrich (St. Louis, Mo.). Promega trypsin gold was purchased from Promega Corporation (Madison, Wis.). Synthetic heavy peptides labeled with ¹³C/¹⁵N on the C-terminal arginine or lysine were purchased from New England Peptide (Gardner, Mass.).

Cell Culture

The MCF10A (MCF7) breast cancer cell line was obtained from the American Type Culture Collection (Manassas, Va.) and was grown in culture media⁶³. Briefly, MCF10A (MCF7) cells were cultured and maintained in 15 cm dishes in ATCC-formulated Eagle's minimum essential medium (Thermo Fisher Scientific) supplemented with 0.01 mg/mL human recombinant insulin and a final concentration of 10% fetal bovine serum (Thermo Fisher Scientific, Waltham, Mass.) with 1% penicillin/streptomycin (Thermo Fisher Scientific). Cells were grown at 37° C. in 95% O₂ and 5% CO₂. Cells were seeded and grown until near confluence.

MCF7 Cell Lysates

MCF7 cells were rinsed twice with ice-cold phosphate-buffered saline (PBS) and harvested in 1 mL of ice-cold PBS containing 1% phosphatase inhibitor cocktail (Pierce, Rockford, Ill.) and 10 mM NaF (Sigma-Aldrich). Cells were centrifuged at 1500 rpm for 10 min at 4° C., and excess PBS was carefully aspirated from the cell pellet. Cell pellets were resuspended in ice-cold cell lysis buffer (250 mM HEPES, 8 M urea, 150 mM NaCl, 1% Triton X-100, pH 6.0) at a ratio of ˜3:1 lysis buffer to cell pellet. Cell lysates were centrifuged at 14,000 rpm at 4° C. for 10 min, and the soluble protein fraction was retained. Protein concentrations were determined by the BCA assay (Pierce).

Fluorescence-Assisted Cell Sorting (FACS) of Single Cells

Prior to cell collection, PCR tubes or 96-well PCR plates were pretreated with 0.1% DDM for coating the surface and later the DDM solution was removed. The pretreated PCR tubes or 96-well PCR plates were air-dried in the fume hood. To avoid cell clumping, after detaching they were dispersed into a single-cell suspension by passing three times through a 25-gauge needle. The cells were suspended in PBS, and pelleted by centrifuging 5 min at 500 g. This process was repeated five times to remove the remaining PBS and trypsin. After that the cells were resuspended in PBS and passed through a 35 μm mesh cap (BD Biosciences, Canaan, CT) to remove large aggregates. A BD Influx flow cytometer (BD Biosciences, San Jose, Calif.) was used to deposit cells into the precoated PCR tubes. Alignment into a Hard-Shell 96-well PCR plate (Bio-Rad, Hercules, Calif.) was done using fluorescent beads (Spherotech, Lake Forest, Ill.), after which the coated PCR tubes were placed into the plates for cell collection. For unstained MCF10A cells, forward and side scatter detectors were used for cell identification. Once sorting gates were established, cells were sorted into the PCR tubes using the 1-drop single sort mode. After isolation of the desired number of cells into the PCR tube, the isolated cells were immediately centrifuged at 1000 g for 10 min at 4° C. to keep the cells at the bottom of the tube to avoid potential cell loss. The PCR tubes with the isolated cells were stored in a −80° C. freezer until further analysis.

Laser Capture Microdissection (LCM) of Tissue Sections

Prior to LCM experiments, a cap of PCR tube was prepopulated with a 5 μL water droplet. Laser capture microdissection (LCM) was performed on a PALM MicroBeam system (Carl Zeiss MicroImaging, Munich, Germany). Voxelation of the tissue section was achieved by selecting the area on the tissue using PalmRobo software, followed by tissue cutting and catapulting. Mouse uterine tissues containing two distinct cell types (luminal epithelium and stroma) were cut at an energy level of 42 and with an iteration cycle of 2 to completely separate 100 μm×100 μm tissue voxels at a thickness of 10 μm. The “CenterRoboLPC” function with an energy level of delta 10 and a focus level of delta 5 was used to catapult tissue voxels into the cap. The “CapCheck” function was activated to confirm successful sample collection from tissue sections to water droplets. After tissue collection into the droplet of the cap, the PCR tube was immediately centrifuged at 1000 g for 10 min at 4° C. to keep collected tissues at the bottom of the tube to avoid potential sample loss. The collected samples were processed directly or stored at −80° C. until use.

PCDX Model Generation and Dissociation of PCDX Tumors and Lungs

The PCDX-205 model was created by implanting prospective CTCs upon lysis of red blood cells (lysis buffer Sigma cat #R7757) and depletion of CD45⁺ PBMCs (Miltenyi Biotec Depletion column cat #130-042-901) from the blood cells of a breast cancer patient (NU-205) into the mammary fat pads of NSG mice. Breast tumors were harvested after 2-3 months and confirmed as a human PCDX with positive expression of human epithelial markers EpCAM, HER2, and CD44 as well as negative expression of mouse H2K^{d}. Tumor cells were lentiviral labeled by L2T⁶⁴ which was generated by using the Luc2 and td Tomato sequences with connection by the short linker, 5′-GGAGATCTAGGAGGTGGAGGTA-GCGGTGGAGGTGGAAGCCAGGATCC-3′ (SEQ ID NO: 8). The L2T gene sequence was removed from a pCDNA3.1⁺ vector and placed within the pFUG lentiviral vector using traditional blunt end cloning. The spontaneous lung metastases were detected by IVIS of the lungs when dissected from the mice.

L2T⁺ PCDX-205 primary tumors and the lungs were harvested and briefly washed in PBS. Tissue was transferred to a Petri dish containing 10 mL dissociation media (RPMI 1640 media with 20 mM HEPES buffer), then minced into fine pieces. 400 μL of Liberase TH enzyme (Roche cat #5401135001) and 100 Units of DNase enzyme (Sigma cat #D4263) were added to the dissociation media, and the Petri dishes containing the tissues were transferred to an incubator at 37° C. and 5% CO₂ for 2 h to complete dissociation. Tissue suspension was mixed every 15 min using a 10 mL serological pipette to aid dissociation. After tissue was completely digested into single cells, the solution was transferred to a 50 mL conical tube. The original petri dish was washed with 15 mL RPMI media containing 2% fetal bovine serum (FBS) (Sigma) and 1% penicillin/streptomycin (Gibco) and the contents transferred to a 50 mL conical tube containing the tissue solution to stop the dissociation reaction. Samples were centrifuged at 300 g for 5 min at 4° C., and the supernatant was removed. Samples were resuspended in 4 mL Red Blood Cell Lysing Buffer (Sigma) and kept on ice for 10 min, after which 20 mL of HBSS (Corning) was added to samples and centrifuged at 300 g for 5 min at 4° C. and the supernatant was removed. Samples were resuspended in 20 mL HBSS and filtered with a 40 μm filter. Cell numbers were counted, and samples were stored on ice until ready for use.

Single Cell Sorting of Patient CTCs from PCDXs and Early Metastases to the Lungs

Cells from dissociated tumor and lung tissues were washed in PBS and then centrifuged at 300 g for 5 min at 4° C. Samples were resuspended in 2% FBS in PBS. MDA-MB-231 cells were collected and suspended in 2% FBS in PBS to serve as a tdTomato (L2T)-negative control for flow analysis. Cancer cells from the tumor and lung samples were sorted based on L2T expression. L2T⁺ tumor cells of the lung metastases were initially sorted into 10% FBS in PBS prior to single cell sorting, and each of the L2T⁺ single cells from the primary tumor and lung metastases was sorted into 5 μL H₂O in a single tube of a 96-tube PCR plate. Plates were sealed, briefly spun on a microplate centrifuge, and stored at −80° C. until later SOP-MS analysis.

Immunohistochemistry Staining

Formalin-fixed and paraffin-embedded tissues were processed and sectioned according to routine protocols. Heat mediated antigen retrieval was used prior to all staining procedures. Tissues were incubated with vimentin antibody (1:200 dilution, clone D21H3, Cell Signaling Technology) or S100A9 antibody (1:100 dilution, provided by Dr. Philippe Tessier at Laval University) overnight at 4° C. Antigen was detected using the EnVision+ Dual Link System (Dako) and counterstained with hematoxylin. Images were taken using a Leica DM4000B microscope and a Leica MC120 HD camera with a 40× objective.

Cell Lysis, Reduction, Alkylation, and Trypsin Digestion

For FACS-isolated cells, 2 μL of 0.1% DDM in 25 mM ammonium bicarbonate (ABC) was added to the PCR tube or each well of the 96-well plate. Intact cells were sonicated at 1-min intervals for 5 times over ice for cell lysis and centrifuged for 3 min at 3000 g. 0.3 μL of 100 mM DTT in 25 mM ABC was added to the PCR tube. Samples were incubated at 75° C. for 1 h for denaturation and reduction. After that, 0.5 μL of 60 mM IAA in 25 mM ABC was added to the PCR tube. Samples were incubated in the dark at room temperature for 30 min for alkylation. The reduction and alkylation steps appear optional: there is no apparent difference in protein identification and quantification between samples with and without reduction and alkylation. 2 of 1 trypsin (Promega) in 25 mM ABC was added to the PCR tube or the 96-well plate at a total amount of 2 ng. Samples were digested for ˜3-4 h at 37° C. with gentle sharking at ˜500 g. After digestion, 0.5 μL of 5% FA was added to the tube to stop enzyme reaction. The final sample volume was adjusted to ˜10-15 μL with the addition of 25 mM ammonium bicarbonate (triethylammonium bicarbonate for TMT samples) for direct LC injection. The sample PCR tube was inserted into the LC vial or the 96-well PCR plate was sealed with a matt. They were either analyzed directly or stored at −20° C. for later LC-MS analysis. For the integrated SOP-BASIL-MS analysis, the digested peptides from single MCF10A cells were labeled with different TMT reagents as sample channels, and 10 ng of peptides from bulk MCF10A cell digests were labeled with TMT126 as the carrier channel. The TMT126 labeled carrier channel peptides were equally distributed to each sample channel, and all the samples were combined together to form one single sample. The combined channel sample was desalted by using a simple reversed phase-based Stage Tip⁶⁵.

For LCM-dissected tissue sections, 1.5 μL of cell lysis buffer containing 0.2% DDM and 5 mM DTT was added to the PCR tube and incubated at 80° C. for 60 min for cell lysis and protein denaturation. IAA was added to the PCR tube with the final concentration of 10 mM. Samples were incubated in the dark at room temperature for 30 min. After that they were diluted by the addition of 25 mM ammonium bicarbonate to reduce the DDM concentration to 0.02%. The mixed Lys-C and trypsin were added to the PCR tube with the final enzyme concentration of 0.5 ng/μL (i.e., a total of 5 ng for the final processing volume of 15 μL). The sample was gently mixed at 850 rpm for 3 min, and then incubated at 37° C. overnight (˜16 h) for digestion. After digestion, 1 μL of 5% FA was added to the PCR tube to stop enzyme reaction. The sample PCR tube was inserted into the LC vial and the sample was either directly analyzed or stored at −20° C. for later LC-MS analysis.

LC-MS/MS Analysis

The single-cell digests were analyzed using a commonly available Q Exactive Plus Orbitrap MS (Thermo Scientific, San Jose, Calif.). The standard LC system consisted of a PAL autosampler (CTC ANALYTICS AG, Zwingen, Switzerland), two Cheminert six-port injection valves (Valco Instruments, Houston, USA), a binary nanoUPLC pump (Dionex UltiMate NCP-3200RS, Thermo Scientific), and an HPLC sample loading pump (1200 Series, Agilent, Santa Clara, USA). Both SPE precolumn (150 μm i.d., 4 cm length) and LC column (50 μm i.d., 70-cm Self-Pack PicoFrit column, New Objective, Woburn, USA) were slurry-packed with 3-μm C18 packing material (300-A pore size) (Phenomenex, Terrence, USA). Sample was fully injected into a 20 μL loop and loaded onto the SPE column using Buffer A (0.1% formic acid in water) at a flow rate of 5 μL/min for 20 min. The concentrated sample was separated at a flow rate of 150 nL/min and a 75 min gradient of 8-35% Buffer B (0.1% formic acid in acetonitrile). The LC column was washed using 80% Buffer B for 10 min and equilibrated using 2% Buffer B for 20 min. Q Exactive Plus Orbitrap MS (Thermo Scientific) was used to analyze the separated peptides. A 2.2 kV high voltage was applied at the ionization source to generate electrospray and ionize peptides. The ion transfer capillary was heated to 250° C. to desolvate droplets. The data dependent acquisition mode was employed to automatically trigger the precursor scan and the MS/MS scans. Precursors were scanned at a resolution of 35,000, an AGC target of 3×10⁶, a maximum ion trap time of 50 ms (100 ms for CTC single cell analysis). Top-10 precursors were isolated with an isolation window of 2, an AGC target of 2×10⁵, a maximum ion injection time of 300 ms (for CTC single-cell analysis, the AGC target of 2×10⁵ and 500 ms ion injection time was used), and fragmented by high energy collision with an energy level of 32%. A dynamic exclusion of 30 s was used to minimize repeated sequencing. MS/MS spectra were scanned at a resolution of 17,500.

Data Analysis

The freely-available open-source MaxQuant software was used for protein identification and quantification. The MS raw files were processed with MaxQuant (Version 1.5.1.11)^{66, 67} and MS/MS spectra were searched by Andromeda search engine against the against a human (or mouse) UniProt database (fasta file dated Apr. 12, 2017) (with the following parameters: tryptic peptides with 0-2 missed cleavage sites; 10 ppm of parent ion tolerance; 0.6 Da of fragment ion mass tolerance; variable modifications (methionine oxidation). Search results were processed with MaxQuant and filtered with a false discovery rate ≤1%. When a peptide library was available, the match between runs (MBR) function was selected to increase proteome coverage. Protein quantification was performed by using the label-free quantitation (LFQ) function. Contaminants were removed from the peptides.txt file prior to use for downstream statistical analysis. Biological functions and signaling pathways were analyzed by using DAVID Bioinformatics Resources (Version 6.8)⁶⁸ and Peruses (Version 1.6.2.1)⁶⁹, and protein-protein association network analysis was performed by the latest version of STRING (Version 11.0)⁷⁰.

Statistics and Reproducibility

At least three biological or technical replicates were used to evaluate reproducibility for sample recovery and SOP-MS. No data exclusion was performed, and no randomization or blinding methods were used in data analysis. After label-free quantification with MaxQuant MBR, the extracted ion chromatogram (XIC) areas of the identified protein groups were log 2 transformed, and then normalized by the median value of each column. The proteins containing at least 50% valid values in one group were kept in the data matrix, and the missing values were imputed by the normal distribution in each column with a width of 0.3 and a downshift of 1.8 by using Perseus (Version 1.6.2.1)⁶⁹. The non-supervised PCA analysis was used to generate PCA plot. The inventors further used Anova t-test to prioritize significantly differentiated proteins (p<0.05, FDR<0.2) for the heatmap generation. The extracted data were further processed and visualized with Microsoft Excel 2017.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

### REFERENCES

1. Wang, Z., Gerstein, M. & Snyder, M. RNA-Seq: a revolutionary tool for transcriptomics. Nat Rev Genet 10, 57-63 (2009).
2. Navin, N. et al. Tumour evolution inferred by single-cell sequencing. Nature 472, 90-94 (2011).
3. Patel, A. P. et al. Single-cell RNA-seq highlights intratumoral heterogeneity in primary glioblastoma. Science 344, 1396-1401 (2014).
4. Bendall, S. C., Nolan, G. P., Roederer, M. & Chattopadhyay, P. K. A deep profiler's guide to cytometry. Trends Immunol 33, 323-332 (2012).
5. Shi, T. J. et al. Advancing the sensitivity of selected reaction monitoring-based targeted quantitative proteomics. Proteomics 12, 1074-1092 (2012).
6. Mertins, P. et al. Reproducible workflow for multiplexed deep-scale proteome and phosphoproteome analysis of tumor tissues by liquid chromatography-mass spectrometry. Nat Protoc 13, 1632-1661 (2018).
7. Mertins, P. et al. Proteogenomics connects somatic mutations to signalling in breast cancer. Nature 534, 55-62 (2016).
8. Zhang, H. et al. Integrated Proteogenomic Characterization of Human High-Grade Serous Ovarian Cancer. Cell 166, 755-765 (2016).
9. Zhang, B. et al. Proteogenomic characterization of human colon and rectal cancer. Nature 513, 382-387 (2014).
10. Wisniewski, J. R., Zougman, A., Nagaraj, N. & Mann, M. Universal sample preparation method for proteome analysis. Nat Methods 6, 359-362 (2009).
11. Kulak, N. A., Pichler, G., Paron, I., Nagaraj, N. & Mann, M. Minimal, encapsulated proteomic-sample processing applied to copy-number estimation in eukaryotic cells. Nat Methods 11, 319-324 (2014).
12. Myers, S. A. et al. Streamlined Protocol for Deep Proteomic Profiling of FAC-sorted Cells and Its Application to Freshly Isolated Murine Immune Cells. Molecular & Cellular Proteomics 18, 995-1009 (2019).
13. Hughes, C. S. et al. Ultrasensitive proteome analysis using paramagnetic bead technology. Molecular Systems Biology 10, 757 (2014).
14. Muller, T. et al. Automated sample preparation with SP3 for low-input clinical proteomics. Mol Syst Biol 16, e9111 (2020).
15. Yamaguchi, H. & Miyazaki, M. Enzyme-immobilized reactors for rapid and efficient sample preparation in MS-based proteomic studies. Proteomics 13, 457-466 (2013).
16. Safdar, M., Spross, J. & Janis, J. Microscale immobilized enzyme reactors in proteomics: Latest developments. J Chromatogr A 1324, 1-10 (2014).
17. Huang, E. L. et al. SNaPP: Simplified Nanoproteomics Platform for Reproducible Global Proteomic Analysis of Nanogram Protein Quantities. Endocrinology 157, 1307-1314 (2016).
18. Lombard-Banek, C., Moody, S. A. & Nemes, P. Single-Cell Mass Spectrometry for Discovery Proteomics: Quantifying Translational Cell Heterogeneity in the 16-Cell Frog (Xenopus) Embryo. Angew Chem Int Ed Engl 55, 2454-2458 (2016).
19. Sun, L. et al. Single Cell Proteomics Using Frog (Xenopus laevis) Blastomeres Isolated from Early Stage Embryos, Which Form a Geometric Progression in Protein Content. Anal Chem 88, 6653-6657 (2016).
20. Saha-Shah, A. et al. Single Cell Proteomics by Data-Independent Acquisition To Study Embryonic Asymmetry in Xenopus laevis. Anal Chem 91, 8891-8899 (2019).
21. Zhu, Y. et al. Nanodroplet processing platform for deep and quantitative proteome profiling of 10-100 mammalian cells. Nat Commun 9, 882 (2018).
22. Shi, T. et al. Facile carrier-assisted targeted mass spectrometric approach for proteomic analysis of low numbers of mammalian cells. Commun Biol 1, 103 (2018).
23. Zhang, P. et al. Carrier-Assisted Single-Tube Processing Approach for Targeted Proteomics Analysis of Low Numbers of Mammalian Cells. Anal Chem 91, 1441-1451 (2019).
24. Shao, X. et al. Integrated Proteome Analysis Device for Fast Single-Cell Protein Profiling. Anal Chem 90, 14003-14010 (2018).
25. Li, Z. Y. et al. Nanoliter-Scale Oil-Air-Droplet Chip-Based Single Cell Proteomic Analysis. Anal Chem 90, 5430-5438 (2018).
26. Budnik, B., Levy, E., Harmange, G. & Slavov, N. SCoPE-MS: mass spectrometry of single mammalian cells quantifies proteome heterogeneity during cell differentiation. Genome Biol 19, 161 (2018).
27. Vitrinel, B., Iannitelli, D. E., Mazzoni, E. O., Christiaen, L. & Vogel, C. Simple Method to Quantify Protein Abundances from 1000 Cells. ACS Omega 5, 15537-15546 (2020).
28. Rauniyar, N. & Yates, J. R., 3rd Isobaric labeling-based relative quantification in shotgun proteomics. J Proteome Res 13, 5293-5309 (2014).
29. Cristofanilli, M. et al. Circulating Tumor Cells, Disease Progression, and Survival in Metastatic Breast Cancer. New England Journal of Medicine 351, 781-791 (2004).
30. Alix-Panabiéres, C. & Pantel, K. Clinical Applications of Circulating Tumor Cells and Circulating Tumor DNA as Liquid Biopsy. Cancer Discovery 6, 479-491 (2016).
31. Aceto, N. et al. Circulating tumor cell clusters are oligoclonal precursors of breast cancer metastasis. Cell 158, 1110-1122 (2014).
32. Gkountela, S. et al. Circulating Tumor Cell Clustering Shapes DNA Methylation to Enable Metastasis Seeding. Cell 176, 98-112 e114 (2019).
33. Liu, X. et al. Homophilic CD44 Interactions Mediate Tumor Cell Aggregation and Polyclonal Metastasis in Patient-Derived Breast Cancer Models. Cancer discovery 9, 96-113 (2019).
34. Mu, Z. et al. Prospective assessment of the prognostic value of circulating tumor cells and their clusters in patients with advanced-stage breast cancer. Breast Cancer Research and Treatment 154, 563-571 (2015).
35. Meng, S. et al. Circulating Tumor Cells in Patients with Breast Cancer Dormancy. Clinical Cancer Research 10, 8152-8162 (2004).
36. Hong, Y., Fang, F. & Zhang, Q. Circulating tumor cell clusters: What we know and what we expect (Review). Int J Oncol 49, 2206-2216 (2016).
37. Mani, S. A. et al. The epithelial-mesenchymal transition generates cells with properties of stem cells. Cell 133, 704-715 (2008).
38. Wang, Y. et al. Vimentin expression in circulating tumor cells (CTCs) associated with liver metastases predicts poor progression-free survival in patients with advanced lung cancer. J Cancer Res Clin Oncol 145, 2911-2920 (2019).
39. Hanahan, D. & Weinberg, R. A. Hallmarks of cancer: the next generation. Cell 144, 646-674 (2011).
40. Padmanaban, V. et al. E-cadherin is required for metastasis in multiple models of breast cancer. Nature 573, 439-444 (2019).
41. Chang, Y. H. et al. New mass-spectrometry-compatible degradable surfactant for tissue proteomics. J Proteome Res 14, 1587-1599 (2015).
42. Masuda, T., Tomita, M. & Ishihama, Y. Phase transfer surfactant-aided trypsin digestion for membrane proteome analysis. J Proteome Res 7, 731-740 (2008).
43. Zhang, X. Less is More: Membrane Protein Digestion Beyond Urea-Trypsin Solution for Next-level Proteomics. Molecular & Cellular Proteomics 14, 2441-2453 (2015).
44. Zhang, X. Instant Integrated Ultradeep Quantitative-structural Membrane Proteomics Discovered Post-translational Modification Signatures for Human Cys-loop Receptor Subunit Bias. Molecular & Cellular Proteomics 15, 3665-3684 (2016).
45. Zhu, Y. et al. Spatially Resolved Proteome Mapping of Laser Capture Microdissected Tissue with Automated Sample Transfer to Nanodroplets. Mol Cell Proteomics 17, 1864-1874 (2018).
46. TruongVo, T. N. et al. Microfluidic channel for characterizing normal and breast cancer cells. J Micromech Microeng 27, article id. 035017 (2017).
47. Crapo, J. D., Barry, B. E., Gehr, P., Bachofen, M. & Weibel, E. R. Cell number and cell characteristics of the normal human lung. Am Rev Respir Dis 126, 332-337 (1982).
48. Nonaka, D., Chiriboga, L. & Rubin, B. P. Differential expression of S100 protein subtypes in malignant melanoma, and benign and malignant peripheral nerve sheath tumors. J Cutan Pathol 35, 1014-1019 (2008).
49. Skliris, G. P. et al. Lesson of the Month—Expression of small breast epithelial mucin (SBEM) protein in tissue microarrays (TMAs) of primary invasive breast cancers. Histopathology 52, 355-369 (2008).
50. Johnson, J. R. et al. IL-22 contributes to TGF-beta1-mediated epithelial-mesenchymal transition in asthmatic bronchial epithelial cells. Respir Res 14, 118 (2013).
51. Ai, J. et al. The role of polymeric immunoglobulin receptor in inflammation-induced tumor metastasis of human hepatocellular carcinoma. J Natl Cancer Inst 103, 1696-1712 (2011).
52. Shiota, M. et al. Hsp27 regulates epithelial mesenchymal transition, metastasis, and circulating tumor cells in prostate cancer. Cancer Res 73, 3109-3119 (2013).
53. Han, L., Jiang, Y., Han, D. & Tan, W. Hsp27 regulates epithelial mesenchymal transition, metastasis and proliferation in colorectal carcinoma. Oncol Lett 16, 5309-5316 (2018).
54. Ohata, T. et al. Fatty acid-binding protein 5 function in hepatocellular carcinoma through induction of epithelial-mesenchymal transition. Cancer Med 6, 1049-1061 (2017).
55. Zhu, Y. et al. Proteomic Analysis of Single Mammalian Cells Enabled by Microfluidic Nanodroplet Sample Preparation and Ultrasensitive NanoLC-MS. Angew Chem Int Ed Engl 57, 12370-12374 (2018).
56. Williams, S. M. et al. Automated Coupling of Nanodroplet Sample Preparation with Liquid Chromatography-Mass Spectrometry for High-Throughput Single-Cell Proteomics. Anal Chem 92, 10588-10596 (2020).
57. Cong, Y. Z. et al. Improved Single-Cell Proteome Coverage Using Narrow-Bore Packed NanoLC Columns and Ultrasensitive Mass Spectrometry. Analytical Chemistry 92, 2665-2671 (2020).
58. Yi, L. et al. Boosting to Amplify Signal with Isobaric Labeling (BASIL) Strategy for Comprehensive Quantitative Phosphoproteomic Characterization of Small Populations of Cells. Anal Chem 91, 5794-5801 (2019).
59. Ibrahim, Y. M. et al. New frontiers for mass spectrometry based upon structures for lossless ion manipulations. Analyst 142, 1010-1021 (2017).
60. Hebert, A. S. et al. Comprehensive Single-Shot Proteomics with FAIMS on a Hybrid Orbitrap Mass Spectrometer. Analytical Chemistry 90, 9529-9537 (2018).
61. Foell, D., Wittkowski, H., Vogl, T. & Roth, J. 5100 proteins expressed in phagocytes: a novel group of damage-associated molecular pattern molecules. J Leukoc Biol 81, 28-37 (2007).
62. Gorges, T. M. et al. Accession of Tumor Heterogeneity by Multiplex Transcriptome Profiling of Single Circulating Tumor Cells. Clin Chem 62, 1504-1515 (2016).
63. Shi, T. et al. Conservation of protein abundance patterns reveals the regulatory architecture of the EGFR-MAPK pathway. Sci Signal 9, rs6 (2016).
64. Liu, H. et al. Cancer stem cells from human breast tumors are involved in spontaneous metastases in orthotopic mouse models. Proc Natl Acad Sci USA 107, 18115-18120 (2010).
65. Rappsilber, J., Mann, M. & Ishihama, Y. Protocol for micro-purification, enrichment, pre-fractionation and storage of peptides for proteomics using StageTips. Nat Protoc 2, 1896-1906 (2007).
66. Cox, J. & Mann, M. MaxQuant enables high peptide identification rates, individualized p.p.b.-range mass accuracies and proteome-wide protein quantification. Nat Biotechnol 26, 1367-1372 (2008).
67. Tyanova, S., Temu, T. & Cox, J. The MaxQuant computational platform for mass spectrometry-based shotgun proteomics. Nat Protoc 11, 2301-2319 (2016).
68. Huang da, W., Sherman, B. T. & Lempicki, R. A. Systematic and integrative analysis of large gene lists using DAVID bioinformatics resources. Nature protocols 4, 44-57 (2009).
69. Tyanova, S. et al. The Perseus computational platform for comprehensive analysis of (prote)omics data. Nature methods 13, 731-740 (2016).
70. Szklarczyk, D. et al. STRING v11: protein-protein association networks with increased coverage, supporting functional discovery in genome-wide experimental datasets. Nucleic Acids Res 47, D607-D613 (2019).
71. Okuda, S. et al. jPOSTrepo: an international standard data repository for proteomes. Nucleic Acids Res 45, D1107-D1111 (2017).

### Supplementary Materials and Methods

Stable Isotope-Labeled Phosphopeptides.

Crude stable isotope-labeled (SIL) phosphopeptides were synthesized with ¹³C/¹⁵N on C-terminal lysine or arginine (New England Peptide, Gardner, Mass.). The peptides were dissolved individually in 15% acetonitrile (ACN) and 0.1% formic acid (FA) at a concentration of 1.5 mM and stored at −80° C. A mixture of these peptides was made with a final concentration of 10 pmol/μL for each peptide.

LC-SRM Analysis.

The SIL phosphopeptides were diluted by ddH₂O into 250 fmol/μL and analyzed using an Altis triple quadruple mass spectrometer (Thermo Fisher Scientific) equipped with a nanoACQUITY UPLC system (Waters, Milford, Mass.) for generating the data of FIG. 6. Peptide samples were loaded onto an ACQUITY UPLC BEH 1.7-μm C18 column (100 μm i.d.×10 cm). The mobile phases were (A) 0.1% FA in water and (B) 0.1% FA in ACN. 2 μL of the sample was loaded onto the column and separated at a flow rate of 400 nL/min using a 72-min gradient as followed (min:% B): 11:0.5, 13.5:10, 17:15, 38:25, 49:38, 50:95, 59:10, 60:95, 64:0.5. The LC column is operated at a temperature of 45° C. The parameters of the instrument were set as follows: Q1 and Q3 resolution were 0.7 fwhm, with 1 s cycle time. Data were acquired in scheduled SRM mode.

The selection of surrogate peptides for epidermal growth factor receptor (EGFR) pathway proteins and the SRM assays were described previously¹. High-purity light peptides (>95%) were used to calibrate crude heavy peptide concentrations. Crude heavy isotope-labeled EGFR pathway peptide standards at a total amount of 30 fmol for each peptide were used for evaluation of peptide recovery with and without DDM (Table 1). Samples were analyzed using a nanoACQUITY UPLC (Waters Corporation, Milford, Mass.) coupled to a TSQ Vantage triple quadrupole mass spectrometer (Thermo Scientific, San Jose, Calif.). The UPLC's nanoACQUITY UPLC BEH 1.7 μm C18 column (75 μm i.d.×20 cm) was connected to a chemically etched 20 μm i.d. fused-silica electrospray emitter via a stainless metal union. Solvents used were 0.1% formic acid in water (mobile phase A) and 0.1% formic acid in 90% acetonitrile (mobile phase B). An amount of ˜12 μL out of the total ˜15 μL peptide sample was directly loaded onto the BEH C18 column from the PCR tube without using a trapping column. Sample loading and separation were performed at a flow rate of 350 and 300 nL/min, respectively. The binary LC gradient was used: 5-20% B in 26 min, 20-25% B in 10 min, 25-40% B in 8 min, 40-95% B in 1 min and at 95% B for 7 min for a total of 52 min, and the analytical column was re-equilibrated at 99.5% A for 8 min. The TSQ Vantage mass spectrometer was operated with ion spray voltages of 2400±100 V, a capillary offset voltage of 35 V, a skimmer offset voltage of −5 V, and a capillary inlet temperature of 220° C. The tube lens voltages were obtained from automatic tuning and calibration without further optimization. The retention time scheduled SRM mode was applied for SRM data collection with the scan window of ≥6 min. The cycle time was set to 1 s, and the dwell time for each transition was automatically adjusted depending on the number of transitions scanned at different retention time windows. A minimal dwell time 10 ms was used for each SRM transition. All the EGFR pathway proteins were simultaneously monitored in a single LC-SRM analysis.

Data analysis. Skyline software was used for all SRM data analysis². The raw data were initially imported into Skyline software for visualization of chromatograms of target peptides to determine the detectability of target peptides. For each peptide the best transition without matrix interference was used for precise quantification. Two criteria were used to determine the peak detection and integration: (1) same retention time and (2) approximately the same relative SRM peak intensity ratios across multiple transitions between endogenous (light) peptide and heavy peptide internal standards. All the data were manually inspected to ensure correct peak detection and accurate integration. The RAW data from TSQ Vantage were loaded into Skyline software to display graphs of extracted ion chromatograms (XICs) of multiple transitions of target proteins monitored.

Background of a PCDX Model.

In the dissemination of metastatic tumors, cancer cells from the primary tumor are shed into the peripheral blood vasculature. These circulating tumor cells (CTCs) serve as the vehicle by which primary tumors can seed distant metastases. In order to become a CTC, cancer cells from the primary tumor must undergo several steps to reach the bloodstream. Initially, tumor cells may undergo an epithelial to mesenchymal transition (EMT) and begin invading the surrounding extracellular matrix and basement membrane³⁻⁵. Eventually tumor cells will reach a local blood vessel and intravasate⁶. CTCs remain in the blood stream for up to several hours as single cells or clusters, sometimes associating with various other cell types, until they extravasate at a potential site of metastasis⁷⁻¹⁰. However, even in patients with advanced metastatic cancers, CTCs are a rare population (normally less than 0.1%) compared to peripheral blood mononuclear cells (PBMCs) within the blood. CTCs are commonly distinguished from other cell populations in the blood by negative expression of CD45, a leukocyte marker, and the positive expression of epithelial markers including EpCAM, cytokeratin, and/or other tumor associated antigens¹¹, which might be heterogeneous and not expressed in all CTCs. There remains understudied concerning the dynamic changes CTCs may undergo compared to tumor cells within the primary tumor and distant metastases. Most notably, CTCs may exhibit cellular junction proteins and properties of cancer stem cells, which promote their ability to cluster and survive in the blood stream and seed distant metastases¹²⁻¹⁶. The detection of CTCs in singles and clusters in patient samples has shown important prognostic value^{7, 14-17}. The characterization of CTC heterogeneity has been impeded due to the difficult sampling and maintenance of this rare population of tumor cells.

The development of patient derived xenografts (PDXs) that develop spontaneous metastases in mice has afforded researchers a representative model system to investigate the molecular and cellular basis of metastasis in vivo^{13, 18}. In this study, the inventors further established patient CTC-derived xenografts (PCDXs) which developed spontaneous lung metastasis, first creation to our knowledge, for single cell proteomic profiling of primary tumor cells as well as spontaneous lung metastases. Lentiviral labeling of this PCDX with the luciferase 2-tdTomato (L2T) dual fusion gene reporter enabled a convenient isolation and FACS-based single cell sorting of L2T⁺ tumor cells from both primary tumor and lung metastasis after dissociation. The single cell proteomic profiling of PCDX model with metastasis not only allowed for the identification of new markers that can be leveraged for CTC isolation, but also facilitated elucidating the heterogeneous alterations of metastatic tumor cells upon colonization of the lungs.

Procedure for Prioritization of the 18 Differentially Expressed Proteins and Generation of the Heatmap:

1) After label-free quantification with MaxQuant MBR, the extracted ion chromatogram (XIC) areas of the identified protein groups were log 2 transformed, and then normalized by the median value of each column; 2) The proteins containing at least 50% valid values in one group were kept in the data matrix, and the missing values were imputed by the normal distribution in each column with a width of 0.3 and a downshift of 1.8 by using Perseus (Version 1.6.2.1); 3) The non-supervised PCA analysis was then used to generate PCA plot; 4) The inventors further used Anova t-test to prioritize significantly differentiated proteins between lung metastatic and primary tumor cells (p<0.05, FDR<0.2) for the heatmap generation.

### SUPPLEMENTARY REFERENCES

1. Shi, T. et al. Conservation of protein abundance patterns reveals the regulatory architecture of the EGFR-MAPK pathway. Sci Signal 9, rs6 (2016).
2. MacLean, B. et al. Skyline: an open source document editor for creating and analyzing targeted proteomics experiments. Bioinformatics 26, 966-968 (2010).
3. Mani, S. A. et al. The epithelial-mesenchymal transition generates cells with properties of stem cells. Cell 133, 704-715 (2008).
4. Wang, Y. et al. Vimentin expression in circulating tumor cells (CTCs) associated with liver metastases predicts poor progression-free survival in patients with advanced lung cancer. Journal of Cancer Research and Clinical Oncology (2019).
5. Hanahan, D. & Weinberg, R. A. Hallmarks of cancer: the next generation. Cell 144, 646-674 (2011).
6. Pantel, K. & Speicher, M. R. The biology of circulating tumor cells. Oncogene 35, 1216-1224 (2016).
7. Cristofanilli, M. et al. Circulating Tumor Cells, Disease Progression, and Survival in Metastatic Breast Cancer. New England Journal of Medicine 351, 781-791 (2004).
8. Mu, Z. et al. Prospective assessment of the prognostic value of circulating tumor cells and their clusters in patients with advanced-stage breast cancer. Breast Cancer Research and Treatment 154, 563-571 (2015).
9. Meng, S. et al. Circulating Tumor Cells in Patients with Breast Cancer Dormancy. Clinical Cancer Research 10, 8152-8162 (2004).
10. Hong, Y., Fang, F. & Zhang, Q. Circulating tumor cell clusters: What we know and what we expect (Review). Int J Oncol 49, 2206-2216 (2016).
11. Paoletti, C. & Hayes, D. F. in Novel Biomarkers in the Continuum of Breast Cancer. (ed. V. Stearns) 235-258 (Springer International Publishing, Cham; 2016).
12. Kreso, A. & Dick, John E. Evolution of the Cancer Stem Cell Model. Cell Stem Cell 14, 275-291 (2014).
13. Liu, H. et al. Cancer stem cells from human breast tumors are involved in spontaneous metastases in orthotopic mouse models. Proc Natl Acad Sci USA 107, 18115-18120 (2010).
14. Liu, X. et al. Homophilic CD44 Interactions Mediate Tumor Cell Aggregation and Polyclonal Metastasis in Patient-Derived Breast Cancer Models. Cancer Discov 9, 96-113 (2019).
15. Aceto, N. et al. Circulating tumor cell clusters are oligoclonal precursors of breast cancer metastasis. Cell 158, 1110-1122 (2014).
16. Gkountela, S. et al. Circulating Tumor Cell Clustering Shapes DNA Methylation to Enable Metastasis Seeding. Cell 176, 98-112 e114 (2019).
17. Alix-Panabiéres, C. & Pantel, K. Clinical Applications of Circulating Tumor Cells and Circulating Tumor DNA as Liquid Biopsy. Cancer Discovery 6, 479-491 (2016).
18. Liu, H. et al. Cancer stem cells from human breast tumors are involved in spontaneous metastases in orthotopic mouse models. Proc Natl Acad Sci USA 107, 18115-18120 (2010).

### Example 3—Experimental Data on Targeted Detection of Wildtype and Mutated Peptides in Cancer Cells Via Mass Spectrometry Using Residual or Minimal Samples

Residual or minimal samples of small numbers of cells from PANC-1 and prostate cancer cell lines were prepared for mass spectrometry and treated with 0.015% DDM. Heavy isotope-labelled standards for peptides of interest were synthesized and used as standards. The inventors demonstrated that the disclosed methods are capable of detecting peptides derived from oncogenes, and single amino acid variants (SAAVs) of said peptides, e.g., SEQ ID NO: 1 (FIG. 13a), SEQ ID NO: 3 (FIG. 13b), SEQ ID NO: 5 (FIG. 14a), and SEQ ID NO: 7 (FIG. 14b). Therefore, the inventors concluded that the disclosed methods are suitable for single-cell proteome analysis, and, further, the methods are suitable for the detection of clinically relevant variant peptides from small or single-cell tissue samples. The detection and quantification of SAVVs from small numbers of cells or single cells may serve as the basis for future translational research in cancer precision medicine and personal immunotherapy and treatment as well as potential target sites for drug design.

TABLE 2
Final BC panel-revised

| Original | | | Mutation | Mutation peptide | Wild type peptide |
|---|---|---|---|---|---|
| No | Gene | Accession | Site | position | position |
| 1 | AR | P10275 | AR- | QLVHM716VK | QLVHV716VK |
| | | | V716M | (SEQ ID NO: 20) | (SEQ ID NO: 21) |
| 2 | BRCA1 | P38398 | BRCA1- | TDAEFVCEW1699TLK | TDAEFVCERI699 |
| | | | R1699W | (SEQ ID NO: 22) | (SEQ ID NO: 23) |
| 3 | BRCA2 | P51587 | BRCA2- | C2660DTEID R | Y2660DTEID R |
| | | | Y2660C | (SEQ ID NO: 24) | (SEQ ID NO: 25) |
| 4 | BRCA2 | P51587 | BRCA2- | TSSGLYIFC2842NER | TSSGLYIFR2842 |
| | | | R2842C | (SEQ ID NO: 26) | (SEQ ID NO: 27) |
| 5 | BRCA2 | P51587 | BRCA2- | LTVD2748QK | LTVG2748QK |
| | | | G2748D | (SEQ ID NO: 28) | (SEQ ID NO: 29) |
| 6 | CCNE1 | P24864 | CCNE1- | L330MVPFAMVIR | W330MVPFAMVIR |
| | | | W330L | (SEQ ID NO: 30) | (SEQ ID NO: 31) |
| 8 | CDK6 | Q00534 | CDK6- | ADQQYECG16AEIGE | ADQQYECV16AEIGEGA |
| | | | V16G | GAYGK | YGK |
| | | | | (SEQ ID NO: 32) | (SEQ ID NO: 33) |
| 9 | FGFR1 | PI 1362 | FGFR1- | NVSFEDAGK338 | NVSFEDAGE338YTCLAGN |
| | | | E338K | (SEQ ID NO: 34) | SIGLSHHSAVVLTVLEALE |
| | | | | | ER |
| | | | | | (SEQ ID NO: 35) |
| 10 | GATA3 | P23771 | GATA3- | NSL390FNPAALSR | NSS390FNPAALSR |
| | | | S390L | (SEQ ID NO: 36) | (SEQ ID NO: 37) |
| 11 | GATA3 | P23771 | GATA3- | VHDSLK383 | VHDSLE383DFPK |
| | | | E383K | (SEQ ID NO: 38) | (SEQ ID NO: 39) |
| 12 | PTEN | P60484 | PTEN- | VAQYPFEN92HNPPQL | VAQYPFED92HNPPQLELIK |
| | | | D92N | ELIK | (SEQ ID NO: 41) |
| | | | | (SEQ ID NO: 40) | |
| 13 | PTEN | P60484 | PTEN- | PFCEDLDQWLSEDDNHV | PFCEDLDQWLSEDDNHVAAI |
| | | | H123Y | AAIY123CK | H123CK |
| | | | | (SEQ ID NO: 42) | (SEQ ID NO: 43) |
| 14 | RB1 | P06400 | RB1- | IMESLAWLSDSPLC570 | IMESLAWLSDSPLF570DLIK |
| | | | F570C | DLIK | (SEQ ID NO: 45) |
| | | | | (SEQ ID NO: 44) | |
| 15 | RB1 | P06400 | RB1- | LP876FDIEGSDEADGSK | |
| | | | R876P | (SEQ ID NO: 46) | |
| 16 | STK11 | Q15831 | STK11- | LV264ENIGK | LF264ENIGK |
| | | | F264V | (SEQ ID NO: 47) | (SEQ ID NO: 48) |
| 17 | ERBB2 | P04626 | ERBB2- | CWGESSEDCQSLTH217TVCA | CWGESSEDCQSLTR217 |
| | | | R217H | GGCAR | (SEQ ID NO: 50) |
| | | | | (SEQ ID NO: 49) | |
| 18 | ERBB2 | P04626 | ERBB2- | CWGESSEDCQSLTC217TVCA | CWGESSEDCQSLTR217 |
| | | | R217C | GGCAR | (SEQ ID NO: 52) |
| | | | | (SEQ ID NO: 51) | |
| 19 | ERBB2 | P04626 | ERBB2- | YTFGASCVTACPYNYLSTDVG | YTFGASCVTACPYNYLSTD |
| | | | S310F | F310CTLVCPLHNQEVTAEDG | VGS310CTLVCPLHNQEVT |
| | | | | TQR | AEDGTQR |
| | | | | (SEQ ID NO: 53) | (SEQ ID NO:54) |
| 20 | ERBB2 | P04626 | ERBB2- | YTFGASCVTACPYNYLSTDVG | YTFGASCVTACPYNYLSTD |
| | | | S310Y | Y310CTLVCPLHNQEVTAEDG | VGS310CTLVCPLHNQEVT |
| | | | | TQR | AEDGTQR |
| | | | | (SEQ ID NO: 55) | (SEQ ID NO: 54) |
| 22 | ERBB2 | P04626 | ERBB2- | LLQETELVEPLTPSGAMPN | LLQETELVEPLTPSGAMPN |
| | | | Q709L | L709AQMR | Q709AQMR |
| | | | | (SEQ ID NO: 56) | (SEQ ID NO: 57) |
| 24 | ERBB2 | P04626 | ERBB2- | | |
| | | | L755S | | |
| 25 | ERBB2 | P04626 | ERBB2- | EM767LDEAYVMAGVGSP | EI767LDEAYVMAGVGSPYVSR |
| | | | I767M | YVSR (SEQ ID NO: 58) | (SEQ ID NO: 59) |
| 26 | ERBB2 | P04626 | ERBB2- | EILH769EAYVMAGVGSPYVSR | EILD769EAYVMAGVGSPYVSR |
| | | | D769H | (SEQ ID NO: 158) | (SEQ ID NO: 59) |
| 27 | ERBB2 | P04626 | ERBB2- | EILY769EAYVMAGVGSPYVSR | EILD769EAYVMAGVGSPYVSR |
| | | | D769Y | (SEQ ID NO: 159) | (SEQ ID NO: 59) |
| 28 | ERBB2 | P04626 | ERBB2- | EILDEAYVMAGL777GSPYVSR | EILDEAYVMAGV777GSPYVSR |
| | | | V777L | (SEQ ID NO: 160) | (SEQ ID NO: 59) |
| 30 | ESR1 | P03372 | ESR1- | VPGFVDLTLHDQVHLLQ380C | VPGFVDLTLHDQVHLLE380C |
| | | | E380Q | AWLEILMIGLVWR | AWLEILMIGLVWR |
| | | | | (SEQ ID NO: 60) | (SEQ ID NO: 61) |
| 31 | ESR1 | P03372 | ESR1- | SIILLNSGVYTFLP463STLK | SIILLNSGVYTFLS463STLK |
| | | | S463P | (SEQ ID NO: 62) | (SEQ ID NO: 63) |
| 32 | ESR1 | P03372 | ESR1- | ITDTLM487HLMAK | ITDTLI487HLMAK |
| | | | I487M | (SEQ ID NO: 64) | (SEQ ID NO: 65) |
| 33 | ESR1 | P03372 | ESR1- | NWPP536YDLLLEMLDAHR | NWPL536YDLLLEMLDAHR |
| | | | L536P | (SEQ ID NO: 66) | (SEQ ID NO: 67) |
| 34 | ESR1 | P03372 | ESR1- | NVVPR536 | NVVPL536YDLLLEMLDAHR |
| | | | L536R | (SEQ ID NO: 68) | (SEQ ID NO: 67) |
| 35 | ESR1 | P03372 | ESR1- | NVVPH536YDLLLEMLDAHR | NVVPL536YDLLLEMLDAHR |
| | | | L536H | (SEQ ID NO: 69) | (SEQ ID NO: 67) |
| 36 | ESR1 | P03372 | ESR1- | NWPLS537DLLLEMLDAHR | NWPLY537DLLLEMLDAHR |
| | | | Y537S | (SEQ ID NO: 70) | (SEQ ID NO: 67) |
| 37 | ESR1 | P03372 | ESR1- | NVVPLN537DLLLEMLDAHR | NVVPLY537DLLLEMLDAHR |
| | | | Y537N | (SEQ ID NO: 71) | (SEQ ID NO: 67) |
| 38 | ESR1 | P03372 | ESR1- | NVVPLC537DLLLEMLDAHR | NVVPLY537DLLLEMLDAHR |
| | | | Y537C | (SEQ ID NO: 72) | (SEQ ID NO: 67) |
| 39 | ESR1 | P03372 | ESR1- | NWPLD537DLLLEMLDAHR | NWPLY537DLLLEMLDAHR |
| | | | Y537D | (SEQ ID NO: 73) | (SEQ ID NO: 67) |
| 40 | ESR1 | P03372 | ESR1- | NVVPLG537DLLLEMLDAHR | N WPLY537DLLLEMLDAHR |
| | | | Y537G | (SEQ ID NO: 74) | (SEQ ID NO: 67) |
| 41 | ESR1 | P03372 | ESR1- | NWPLH537DLLLEMLDAHR | NWPLY537DLLLEMLDAHR |
| | | | Y537H | (SEQ ID NO: 75) | (SEQ ID NO: 67) |
| 42 | ESR1 | P03372 | ESR1- | NWPLYG538LLLEMLDAHR | NWPLYD538LLLEMLDAHR |
| | | | D538G | (SEQ ID NO: 76) | (SEQ ID NO: 67) |
| 45 | PIK33CA | P42336 | PIK33CA- | EA81FFDETR | EE81FFDETR |
| | | | E81A | (SEQ ID NO: 77) | (SEQ ID NO: 78) |
| 46 | PIK3CA | P42336 | PIK3CA- | Q88LCDLR | 88LCDLR |
| | | | R88Q | (SEQ ID NO: 79) | (SEQ ID NO: 80) |
| 47 | PIK3CA | P42336 | PIK3CA- | LCDLQ93LFQPFLK | LCDLR93 |
| | | | R93Q | (SEQ ID NO: 81) | (SEQ ID NO: 80) |
| 48 | PIK3CA | P42336 | PIK3CA- | VIEPVV106NR | VIEPVG106NR |
| | | | G106V | (SEQ ID NO: 82) | (SEQ ID NO: 83) |
| 49 | PIK3CA | P42336 | PIK3CA- | VIEPVGT107R | VIEPVGN107R |
| | | | N107T | (SEQ ID NO: 84) | (SEQ ID NO: 83) |
| 50 | PIK3CA | P42336 | PIK3CA- | VIEPVGNH108EEK | VIEPVGNR108 |
| | | | R108H | (SEQ ID NO: 85) | (SEQ ID NO: 83) |
| 51 | PIK3CA | P42336 | PIK3CA- | EID118FAIGMPVCEFDMVK | EIG118FAIGMPVCEFD |
| | | | G118D | (SEQ ID NO: 86) | MVK |
| | | | | | (SEQ ID NO: 87) |
| 52 | PIK33CA | P42336 | PIK3CA- | ILCATYVK345 | ILCATYVN345VNIR |
| | | | N345K | (SEQ ID NO: 88) | (SEQ ID NO: 89) |
| 52 | PIK3CA | P42336 | PIK3CA- | TGIYHGGK365 | TGIYHGGE365PLCDNVNTQR |
| | | | E365K | (SEQ ID NO: 90) | (SEQ ID NO: 91) |
| 54 | PIK3CA | P42336 | PIK3CA- | EEHR420 | EEHC420PLAWGNINLFDYT |
| | | | C420R | (SEQ ID NO: 92) | DTLVSGK |
| | | | | | (SEQ ID NO: 93) |
| 56 | PIK3CA | P42336 | PIK3CA- | MALNLWPVPHGLK453 | MALNLWPVPHGLE453DLLNP |
| | | | E453K | (SEQ ID NO: 94) | IGVTGSNPNK |
| | | | | | (SEQ ID NO: 95) |
| 60 | PIK3CA | P42336 | PIK3CA- | DPLSK542 | DPLSE542ITEQEK |
| | | | E542K | (SEQ ID NO: 96) | (SEQ ID NO: 97) |
| 61 | PIK3CA | P42336 | PIK3CA- | DPLSQ542ITEQEK | DPLSE542ITEQEK |
| | | | E542Q | (SEQ ID NO: 98) | (SEQ ID NO: 97) |
| 62 | PIK3CA | P42336 | PIK3CA- | DPLSEITK545 | DPLSEITE545QEK |
| | | | E545K | (SEQ ID NO: 99) | (SEQ ID NO: 97) |
| 63 | PIK3CA | P42336 | PIK3CA- | DPLSEITQ545QEK | DPLSEITE545QEK |
| | | | E5450 | (SEQ ID NO: 100) | (SEQ ID NO: 97) |
| 64 | PIK33CA | P42336 | PIK3CA- | DPLSEITG545QEK | DPLSEITE545QEK |
| | | | E545G | (SEQ ID NO: 101) | (SEQ ID NO: 97) |
| 65 | PIK3CA | P42336 | PIK3CA- | DPLSEITER546 | DPLSEITEQ546EK |
| | | | Q546R | (SEQ ID NO: 102) | (SEQ ID NO: 97) |
| 66 | PIK3CA | P42336 | PIK3CA- | DPLSEITEK546 | DPLSEITEQ546EK |
| | | | Q546K | (SEQ ID NO: 103) | (SEQ ID NO: 97) |
| 67 | PIK3CA | P42336 | PIK3CA- | DK726 | DE726TQK |
| | | | E726K | | (SEQ ID NO: 104) |
| 68 | PIK3CA | P42336 | PIK3CA- | SCAGYCVATFILGIE914 | SCAGYCVATFILGIG914DR |
| | | | G914E | DR | (SEQ ID NO: 106) |
| | | | | (SEQ ID NO: 105) | |
| 70 | PIK3CA | P42336 | PIK33CA- | S1025LALDK | T1025LALDK |
| | | | T1025S | (SEQ ID NO: 107) | (SEQ ID NO: 108) |
| 71 | PIK3CA | P42336 | PIK3CA- | TLALN1029K | TLALD1029K |
| | | | D1029N | (SEQ ID NO: 109) | (SEQ ID NO: 108) |
| 72 | PIK3CA | P42336 | PIK3CA- | TEQEALK1037 | TEQEALE1037YFMK |
| | | | E1037K | (SEQ ID NO: 110) | (SEQ ID NO: 111) |
| 74 | PIK3CA | P42336 | PIK3CA- | QMNDAR1047 | QMNDAH1047HGGWTTK |
| | | | H1047R | (SEQ ID NO: 112) | (SEQ ID NO: 113) |
| 75 | PIK3CA | P42336 | PIK3CA- | QMNDAL1047HGGWTTK | QMNDAH1047HGGWTTK |
| | | | H1047L | (SEQ ID NO: 114) | (SEQ ID NO: 113) |
| 76 | PIK3CA | P42336 | PIK3CA- | QMNDAQ1047HGGWTTK | QMNDAH1047HGGWTTK |
| | | | H10470 | (SEQ ID NO: 115) | (SEQ ID NO: 113) |
| 77 | PIK3CA | P42336 | PIK3CA- | QMNDAHHR1049 | QMNDAHHG1049GWTTK |
| | | | GI049R | (SEQ ID NO: 116) | (SEQ ID NO: 113) |
| 78 | TP53 | P04637 | TP53- | SVTCTN126SPALNK | SVTCTY126SPALNK |
| | | | Y126N | (SEQ ID NO: 117) | (SEQ ID NO: 118) |
| 79 | TP53 | P04637 | TP53- | SVTCTYSPALNR132 | SVTCTYSPALNK132 |
| | | | KI32R | (SEQ ID NO: 119) | (SEQ ID NO: 118) |
| 80 | TP53 | P04637 | TP53- | SVTCTYSPALNE132MFCQ | SVTCTYSPALNK132 |
| | | | K132E | LAK | (SEQ ID NO: 118) |
| | | | | (SEQ ID NO: 120) | |
| 81 | TP 5 3 | P04637 | TP53- | MFY135QLAK | MFC1350LAK |
| | | | C135Y | (SEQ ID NO: 121) | (SEQ ID NO: 122) |
| 82 | TP53 | P04637 | TP53- | TCPM143QLWVDSTPPPGTR | TCPV143QLWVDSTPPPGTR |
| | | | V143M | (SEQ ID NO: 123) | (SEQ ID NO: 124) |
| 83 | TP53 | P04637 | TP53- | TCPVQLWVDSTS151PPGTR | TCPVQLWVDSTP151PPGTR |
| | | | P151S | (SEQ ID NO: 173) | (SEQ ID NO: 124) |
| 84 | TP53 | P04637 | TP53- | TCPVQLWVDSTH151PPGTR | TCPVQLWVDSTP151PPGTR |
| | | | P151H | (SEQ ID NO: 174) | (SEQ ID NO: 124) |
| 85 | TP53 | P04637 | TP53- | TCPVQLWVDSTPL152PGTR | TCPVQLWVDSTPP152PGTR |
| | | | P152L | (SEQ ID NO: 175) | (SEQ ID NO: 124) |
| 86 | TP53 | P04637 | TP53- | TCPVQLWVDSTPPPGI155R | TCPVQLWVDSTPPPGT155R |
| | | | T155I | (SEQ ID NO: 176) | (SEQ ID NO: 124) |
| 87 | TP53 | P04637 | TP53- | TCPVQLWVDSTPPPGTP156 | TCPVQLWVDSTPPPGTR156 |
| | | | RI56P | VR | (SEQ ID NO: 124) |
| | | | | (SEQ ID NO: 177) | |
| 88 | TP53 | P04637 | TP53- | QSQHMTEF172VR | QSQHMTEV172VR |
| | | | V172F | (SEQ ID NO: 125) | (SEQ ID NO: 126) |
| 89 | TP53 | P04637 | TP53- | QSQHMTEVM173R | QSQHMTEW173R |
| | | | V173M | (SEQ ID NO: 127) | (SEQ ID NO: 126) |
| 93 | TP53 | P04637 | TP53- | CSDSDGLAPPQL193LIR | CSDSDGLAPPQH193LIR |
| | | | H193L | (SEQ ID NO: 128) | (SEQ ID NO: 129) |
| 106 | TP53 | P04637 | TP53- | S249PILTIITLEDSS | (R)249PILTIITLED |
| | | | R249S | GNLLGR | SSGNLLGR |
| | | | | (SEQ ID NO: 130) | (SEQ ID NO: 131 |
| 107 | TP53 | P04637 | TP53- | NSL270EVR | NSF270EVR |
| | | | F270L | (SEQ ID NO: 132) | (SEQ ID NO: 133) |
| 108 | TP53 | P04637 | TP53- | NSFEVH273VCACPGR | NSFEVR273 |
| | | | R273H | (SEQ ID NO: 134) | (SEQ ID NO: 133) |
| 109 | TP53 | P04637 | TP53- | NSFEVC273VCACPGR | NSFEVR273 |
| | | | R273C | (SEQ ID NO: 135) | (SEQ ID NO: 133) |
| 111 | TP53 | P04637 | TP53- | VCACPGG280DR | VCACPGR280 |
| | | | R280G | (SEQ ID NO: 136) | (SEQ ID NO: 137) |
| 113 | TP53 | P04637 | TP53- | TEV286ENLR | TEE286ENLR |
| | | | E286V | (SEQ ID NO: 138) | (SEQ ID NO: 139) |
| | KRAS | P01116 | KRAS- | LVVVGAG12GVGK | LVVVGAG12GVGK |
| | | | G12D | (SEQ ID NO: 140) | (SEQ ID NO: 140) |
| | KRAS | P01116 | KRAS- | LVWGAV12GVGK | LVWGAG12GVGK |
| | | | G12V | (SEQ ID NO: 141) | (SEQ ID NO: 140) |
| | KRAS | P01116 | KRAS- | LVWGAC12GVGK | LVWGAG12GVGK |
| | | | G12C | (SEQ ID NO: 142) | (SEQ ID NO: 140) |
| | KRAS | P01116 | KRAS- | LVVVGAA12GVGK | LVVVGAG12GVGK |
| | | | G12A | (SEQ ID NO: 143) | (SEQ ID NO: 140) |
| | KRAS | P01116 | KRAS- | LVWGAR12 | LVWGAG12GVGK |
| | | | G12R | (SEQ ID NO: 144) | (SEQ ID NO: 140) |
| | KRAS | PI) 1116 | KRAS- | LVVVGAGV13VGK | LVVVGAGG13VGK |
| | | | G13D | (SEQ ID NO: 145) | (SEQ ID NO: 146) |
| | KRAS | P01116 | KRAS- | LVVVGAGGI14GK | LVVVGAGGV14GK |
| | | | V14I | (SEQ ID NO: 147) | (SEQ ID NO: 147) |
| | KRAS | P01116 | KRAS- | QVVIDGETCLLDILDT | QVVIDGETCLLDILDTA |
| | | | 061H | AGH61EEYSAMR | GQ61EEYSAMR |
| | | | | (SEQ ID NO: 148) | (SEQ ID NO: 149) |
| | KRAS | P01116 | KRAS- | DSEDVPMVLVGNN117C | DSEDVPMVLVGNK117 |
| | | | K117N | DLPSR | (SEQ ID NO: 161) |
| | | | | (SEQ ID NO: 150) | |
| | KRAS | P01116 | KRAS- | SYGIPFIETST146K | SYGIPFIETSA146K |
| | | | A146T | (SEQ ID NO: 152) | (SEQ ID NO: 153) |
| | EGFR | P00533 | EGFR- | LLGICLTSTVQLIM790Q | LLGICLTSTVQLIT790Q |
| | | | T790M | LMPFGCLLDYVR | LMPFGCLLDYVR |
| | | | | (SEQ ID NO: 154) | (SEQ ID NO: 155) |
| | EGFR | P00533 | EGFR- | ITDFGR858 | ITDFGL858AK |
| | | | L858R | (SEQ ID NO: 156) | (SEQ ID NO: 157) |

TABLE 3
Final BC panel

| Remark | |
|---|---|
| (Mutation | |
| peptide) | Wild type peptide position |
| M | QLVHV716VK |
| | (SEQ ID NO: 21) |
| C | TDAEFVCER1699 |
| | (SEQ ID NO: 23) |
| c | Y2660DTEIDR |
| | (SEQ ID NO: 25) |
| c | TSSGLYIFR2842 |
| | (SEQ ID NO: 27) |
| short (6 | LTVG2748QK |
| aa) | (SEQ ID NO: 29) |
| 2M | W330MVPFAMVIR |
| | (SEQ ID NO: 31) |
| C | ADQQYECV16AEIGEGAYGK |
| | (SEQ ID NO: 33) |
| | NVSFEDAGE338YTCLAGNS |
| | IGLSHHSAWLTVLEALEER |
| | (SEQ ID NO: 35) |
| | NSS390FNPAALSR |
| | (SEQ ID NO: 37) |
| Short | VHDSLE383DFPK |
| (6 aa) | (SEQ ID NO: 39) |
| | VAQYPFED92HNPPQLELIK |
| | (SEQ ID NO: 41) |
| 2C | PFCEDLDQWLSEDDNHVAAIH123CK |
| | (SEQ ID NO: 43) |
| M, C | IMESLAWLSDSPLF570DLIK |
| | (SEQ ID NO: 45) |
| | LF264ENIGK |
| | (SEQ ID NO: 48) |
| 4C | CWGESSEDCQSLTR217 |
| | (SEQ ID NO: 50) |
| 5C | CWGESSEDCQSLTR217 |
| | (SEQ ID NO: 50) |
| long (42 | YTFGASCVTACPYNYLSTDVGS310C |
| aa) | TLVCPLHNQEVTAEDGTQR |
| | (SEQ ID NO: 54) |
| long (42 | YTFGASCVTACPYNYLSTDVGS310C |
| aa) | TLVCPLHNQEVTAEDGTQR |
| | (SEQ ID NO: 54) |
| M | LLQETELVEPLTPSGAMPNQ709AQMR |
| | (SEQ ID NO: 57) |
| short (3 | VL755R |
| aa) | |
| M | EI767LDEAYVMAGVGSPYVSR |
| | (SEQ ID NO: 59) |
| M | EILD769EAYVMAGVGSPYVSR |
| | (SEQ ID NO: 59) |
| M | EILD769EAYVMAGVGSPYVSR |
| | (SEQ ID NO: 59) |
| M | EILDEAYVMAGV777GSPYVSR |
| | (SEQ ID NO: 59) |
| long (31 | VPGFVDLTLHDQVHLLE380C |
| aa) | AWLEILMIGLVWR |
| | (SEQ ID NO: 61) |
| | SIILLNSGVYTFLS463STLK |
| | (SEQ ID NO: 63) |
| M | ITDTLI487HLMAK |
| | (SEQ ID NO: 65) |
| M | NWPL536YDLLLEMLDAHR |
| | (SEQ ID NO: 67) |
| short (5 | NWPL536YDLLLEMLDAHR |
| aa) | (SEQ ID NO: 67) |
| M | NWPL536YDLLLEMLDAHR |
| | (SEQ ID NO: 67) |
| M | NWPLY537DLLLEMLDAHR |
| | (SEQ ID NO: 67) |
| M | NWPLY537DLLLEMLDAHR |
| | (SEQ ID NO: 67) |
| M | NWPLY537DLLLEMLDAHR |
| | (SEQ ID NO: 67) |
| M | NVVPLY537DLLLEMLDAHR |
| | (SEQ ID NO: 67) |
| M | NWPLY537DLLLEMLDAHR |
| | (SEQ ID NO: 67) |
| M | NWPLY537DLLLEMLDAHR |
| | (SEQ ID NO: 67) |
| M | NWPLYD538LLLEMLDAHR |
| | (SEQ ID NO: 67) |
| | EE81FFDETR |
| | (SEQ ID NO: 78) |
| short (6 | 88LCDLR |
| aa) | (SEQ ID NO: 80) |
| C | LCDLR93 |
| | (SEQ ID NO: 80) |
| | VIEPVG106NR |
| | (SEQ ID NO: 83) |
| | VIEPVGN107R |
| | (SEQ ID NO: 83) |
| | VIEPVGNR108 |
| | (SEQ ID NO: 83) |
| C | EIG118FAIGMPVCEFDMVK |
| | (SEQ ID NO: 87) |
| C | ILCATYVN345VNIR |
| | (SEQ ID NO: 89) |
| OK | TGIYHGGE365PLCDNVNTOR |
| | (SEQ ID NO: 91) |
| short (4 | EEHC420PLAWGNINLFDYT |
| aa) | DTLVSGK |
| | (SEQ ID NO: 93) |
| M | MALNLWPVPHGLE453DLLNP |
| | IGVTGSNPNK |
| | (SEQ ID NO: 95) |
| short (5 | DPLSE542ITEQEK |
| aa) | (SEQ ID NO: 97) |
| | DPLSE542ITEQEK |
| | (SEQ ID NO: 97) |
| | DPLSEITE545QEK |
| | (SEQ ID NO: 97) |
| | DPLSEITE545QEK |
| | (SEQ ID NO: 97) |
| | DPLSEITE545QEK |
| | (SEQ ID NO: 97) |
| | DPLSEITEQ546EK |
| | (SEQ ID NO: 97) |
| | DPLSEITE0546EK |
| | (SEQ ID NO: 97) |
| short (2 | DE726TQK |
| aa) | (SEQ ID NO: 104) |
| 2C | SCAGYCVATFILGIG914DR |
| | (SEQ ID NO: 106) |
| short (6 | T1025LALDK |
| aa) | (SEQ ID NO: 108) |
| short (6 | TLALD1029K |
| aa) | (SEQ ID NO: 108) |
| OK | TEOEALE1037YFMK |
| | (SEQ ID NO: 111) |
| short (6 | OMNDAH1047HGGWTTK |
| aa) | (SEQ ID NO: 113) |
| M | QMNDAH1047HGGWTTK |
| | (SEQ ID NO: 113) |
| M | QMNDAH1047HGGWTTK |
| | (SEQ ID NO: 113) |
| M | OMNDAHHG1049GWTTK |
| | (SEQ ID NO: 113) |
| C | SVTCTY126SPALNK |
| | (SEQ ID NO: 118) |
| C | SVTCTYSPALNK132 |
| | (SEQ ID NO: 118) |
| 2C, M | SVTCTYSPALNK132 |
| | (SEQ ID NO: 118) |
| M | MFC135QLAK |
| | (SEQ ID NO: 122) |
| C, M | TCPV143QLWVDSTPPPGTR |
| C | TCPVQLWVDSTP151PPGTR |
| c | TCPVQEWVDSTP151PPGTR |
| c | TCPVQLWVDSTPP152PGTR |
| c | TCPVQLWVDSTPPPGT155R |
| c | TCPVQLWVDSTPPPGTR156 |
| M | QSQHMTEV172VR |
| | (SEQ ID NO: 126) |
| M | QSQHMTEW173R |
| | (SEQ ID NO: 126) |
| C | CSDSDGLAPPQH193LIR |
| | (SEQ ID NO: 129) |
| | (R)249PILTIITLEDSSGNLLGR |
| | (SEQ ID NO: 131) |
| short (6 | NSF270EVR |
| aa) | (SEQ ID NO: 133) |
| 2C | NSFEVR273 |
| | (SEQ ID NO: 133) |
| 3C | NSFEVR273 |
| | (SEQ ID NO: 133) |
| 2C | VCACPGR280 |
| | (SEQ ID NO: 137) |
| | TEE286ENLR |
| | (SEQ ID NO: 139) |

TABLE 4
Panel-selected 44 sites

| Mutation peptide | Wild type peptide | |
|---|---|---|
| sequence Filtered | sequence Filtered | Remark |
| NSLFNPAALSR | NSSFNPAALSR | |
| (SEQ ID NO: 36) | (SEQ ID NO: 37) | |
| VAQYPFENHNPPQLE | VAQYPFEDHNPPQLE | Wild type |
| LIK | LIK | detected in |
| (SEQ ID NO: 40) | (SEQ ID NO: 41) | lung cancer |
| | | in-house |
| | | data |
| LVENIGK | LFENIGK | Wild type |
| (SEQ ID NO: 47) | (SEQ ID NO: 48) | detected in |
| | | lung cancer |
| | | in-house |
| | | data |
| SIILLNSGVYTFLPS | SIILLNSGVYTFLSS | |
| TLK | TLK | |
| (SEQ ID NO: 62) | (SEQ ID NO: 63) | |
| EAFFDETR | EEFFDETR | |
| (SEQ ID NO: 77) | (SEQ ID NO: 78) | |
| VIEPVVNR | VIEPVGNR | |
| (SEQ ID NO: 82) | (SEQ ID NO: 83) | |
| VIEPVGTR | VIEPVGNR | |
| (SEQ ID NO: 84) | (SEQ ID NO: 83) | |
| VIEPVGNHEEK | VIEPVGNR | |
| (SEQ ID NO: 85) | (SEQ ID NO: 83) | |
| DPLSQITEQEK | DPLSEITEQEK | Wild type |
| (SEQ ID NO: 96) | (SEQ ID NO: 97) | detected in |
| | | lung cancer |
| | | in-house |
| | | data |
| DPLSEITK | DPLSEITEQEK | Wild type |
| (SEQ ID NO: 99) | (SEQ ID NO: 97) | detected in |
| | | lung cancer |
| | | in-house |
| | | data |
| DPLSEITQQEK | DPLSEITEQEK | Wild type |
| (SEQ ID NO: 100) | (SEQ ID NO: 97) | detected in |
| | | lung cancer |
| | | in-house |
| | | data |
| DPLSEITGQEK | DPLSEITEQEK | Wild type |
| (SEQ ID NO: 101) | (SEQ ID NO: 97) | detected in |
| | | lung cancer |
| | | in-house |
| | | data |
| DPLSEITER | DPLSEITEQEK | Wild type |
| (SEQ ID NO: 102) | (SEQ ID NO: 97) | detected in |
| | | lung cancer |
| | | in-house |
| | | data |
| DPLSEITEK | DPLSEITEQEK | Wild type |
| (SEQ ID NO: 103) | (SEQ ID NO: 97) | detected in |
| | | lung cancer |
| | | in-house |
| | | data |
| SPILTIITLEDSSGNLLGR | PILTIITLEDSSGNLLGR | Wild type |
| (SEQ ID NO: 130) | (SEQ ID NO: 131) | detected in |
| | | lung cancer |
| | | in-house |
| | | data |
| TEVENLR | TEEENLR | |
| (SEQ ID NO: 138) | (SEQ ID NO: 139) | |
| QLVHMVK | QLVHVVK | |
| (SEQ ID NO: 20) | (SEQ ID NO: 21) | |
| LMVPFAMV1R | WMVPFAMVIR | |
| (SEQ ID NO: 30) | (SEQ ID NO: 31) | |
| LLQETELVEPLTPSG | LLQETELVEPLTPSG | Wild type |
| AMPNLAQMR | AMPNQAQMR | detected in |
| (SEQ ID NO: 56) | (SEQ ID NO: 57) | lung cancer |
| | | in-house |
| | | data |
| EMLDEAYVMAGVGSP | EILDEAYVMAGVGSP | Wild type |
| YVSR | YVSR | detected in |
| (SEQ ID NO: 66) | (SEQ ID NO: 67) | lung cancer |
| | | in-house |
| | | data |
| EILHEAYVMAGVGSP | EILDEAYVMAGVGSP | Wild type |
| YVSR | YVSR | detected in |
| (SEQ ID NO: 158) | (SEQ ID NO: 67) | lung cancer |
| | | in-house |
| | | data |
| EILYEAYVMAGVGSP | EILDEAYVMAGVGSP | Wild type |
| YVSR | YVSR | detected in |
| (SEQ ID NO: 159) | (SEQ ID NO: 67) | lung cancer |
| | | in-house |
| | | data |
| EILDEAYVMAGLGSP | EILDEAYVMAGVGSP | Wild type |
| YVSR | YVSR | detected in |
| (SEQ ID NO: 160) | (SEQ ID NO: 67) | lung cancer |
| | | in-house |
| | | data |
| ITDTLMHLMAK | ITDTLIHLMAK | |
| (SEQ ID NO: 64) | (SEQ ID NO: 65) | |
| NVVPPYDLLLEMLDAHR | NVVPLYDLLLEMLDAHR | |
| (SEQ ID NO: 66) | (SEQ ID NO: 67) | |
| NVVPHYDLLLEMLDAHR | NVVPLYDLLLEMLDAHR | |
| (SEQ ID NO: 69) | (SEQ ID NO: 67) | |
| NVVPLSDLLLEMLDAHR | NVVPLYDLLLEMLDAHR | |
| (SEQ ID NO: 70) | (SEQ ID NO: 67) | |
| NVVPLNDLLLEMLDAHR | NVVPLYDLLLEMLDAHR | |
| (SEQ ID NO: 71) | (SEQ ID NO: 67) | |
| NVVPLCDLLLEMLDAHR | NVVPLYDLLLEMLDAHR | |
| (SEQ ID NO: 72) | (SEQ ID NO: 67) | |
| NVVPLDDLLLEMLDAHR | NVVPLYDLLLEMLDAHR | |
| (SEQ ID NO: 73) | (SEQ ID NO: 67) | |
| NVVPLGDLLLEMLDAHR | NVVPLYDLLLEMLDAHR | |
| (SEQ ID NO: 74) | (SEQ ID NO: 67) | |
| NVVPLHDLLLEMLDAHR | NVVPLYDLLLEMLDAHR | |
| (SEQ ID NO: 75) | (SEQ ID NO: 67) | |
| NVVPLYGLLLEMLDAHR | NVVPLYDLLLEMLDAHR | |
| (SEQ ID NO: 76) | (SEQ ID NO: 67) | |
| MALNLWPVPHVLEDLL | MALNLWPVPHGLEDLL | Wild type |
| NPIGVTGSNPNK | NPIGVTGSNPNK | detected in |
| (SEQ ID NO: 161) | (SEQ ID NO: 95) | lung cancer |
| | | in-house |
| | | data |
| MALNLWPVPHGLK | MALNLWPVPHGLEDLL | Wild type |
| (SEQ ID NO: 94) | NPIGVTGSNPNK | detected in |
| | (SEQ ID NO: 95) | lung cancer |
| | | in-house |
| | | data |
| MALNLWPVPHGLQDL | MALNLWPVPHGLEDLLN | Wild type |
| LNPIGVIGSNPNK | PIGVTGSNPNK | detected in |
| (SEQ ID NO: 162) | (SEQ ID NO: 95) | lung cancer |
| | | in-house |
| | | data |
| MALNLWPVPHGLGDL | MALNLWPVPHGLEDLLN | Wild type |
| LNPIGVTGSNPNK | PIGVTGSNPNK | detected in |
| (SEQ ID NO: 163) | (SEQ ID NO: 95) | lung cancer |
| | | in-house |
| | | data |
| QHANLFINLFSIMLG | QHANLFINLFSMMLGSG | |
| SGMPELQSFDDIAYIR | MPELQSFDDIAYIR | |
| (SEQ ID NO: 164) | (SEQ ID NO: 165) | |
| TEQEALK | TEQEALEYFMK | Wild type |
| (SEQ ID NO: 110) | (SEQ ID NO: 111) | detected in |
| | | lung cancer |
| | | in-house |
| | | data |
| QMNDALHGGWTTK | QMNDAHHGGWTTK | |
| (SEQ ID NO: 114) | (SEQ ID NO: 113) | |
| QMNDAQHGGWTTK | QMNDAHHGGWTTK | |
| (SEQ ID NO: 115) | (SEQ ID NO: 113) | |
| QMNDAHHR | QMNDAHHGGWTTK | |
| (SEQ ID NO: 116) | (SEQ ID NO: 113) | |
| QSQHMTEFVR | QSQHMTEVVR | Wild type |
| (SEQ ID NO: 125) | (SEQ ID NO: 126) | detected in |
| | | lung cancer |
| | | in-house |
| | | data |
| QSQHMTEVMR | QSQHMTEVVR | Wild type |
| (SEQ ID NO: 127) | (SEQ ID NO: 126) | detected in |
| | | lung cancer |
| | | in-house |
| | | data |

Note:
More stringent criteria from Reta

TABLE 5
Panel_peptides (all detail)

| | | | | | | | Remark |
|---|---|---|---|---|---|---|---|
| | | | | Mutation | Remark | Wild type | (Wild |
| Original | | | Mutation | peptide | (Mutation | Peptide | type |
| No | Gene | Accession | Site | position | peptide) | position | peptide) |
| 1 | AR | P10275 | AR-V716M | QLVHM716VK | M | QLVHV716VK | |
| | | | | (SEQ ID NO: 20) | | (SEQ ID NO: 21) | |
| 2 | BRCA1 | P38398 | BRCA1- | TDAEFVCEW1699TLK | C | TDAEFVCER1699 | C |
| | | | R1699W | (SEQ ID NO: 22) | | (SEQ ID NO: 23) | |
| 3 | BRCA2 | P51587 | BRCA2- | C2660DTEIDR | c | Y2660DTEIDR | |
| | | | Y2660C | (SEQ ID NO: 24) | | (SEQ ID NO: 25) | |
| 4 | BRCA2 | P51587 | BRCA2- | TSSGLYIFC2842N | c | TSSGLYIFR2842 | |
| | | | R2842C | ER | | (SEQ ID NO: 27) | |
| | | | | (SEQ ID NO: 25) | | | |
| 5 | BRCA2 | P51587 | BRCA2- | LTVD2748QK | short | LTVG2748QK | short (6 |
| | | | G2748D | (SEQ ID NO: 28) | (6 aa) | (SEQ ID NO: 29) | aa) |
| 6 | CCNE1 | P24864 | CCNEI-W330L | L330MVPFAMVIR | 2M | W330MVPFAMVIR | 2M |
| | | | | (SEQ ID NO: 30) | | (SEQ ID NO: 31) | |
| 7 | CCNE1 | P24864 | CCNE1-Y296H | LSPLTIVSWLNVY | long | LSPLTIVSWLNVYM | long (76 |
| | | | | MQVAYLNDLHEVL | (76 aa) | QVAYLNDLHEVLLP | aa) |
| | | | | LPQYPQQIFIQIA | | QYPQQIFIQ1AELL | |
| | | | | ELLDLCVLDVDCL | | DLCVLDVDCLEFP | |
| | | | | EFPH296GILAAS | | Y296GILAASALY | |
| | | | | ALYHFSSSELMQK | | HFSSSELMQK | |
| | | | | (SEQ ID NO: 166) | | (SEQ ID NO: 167) | |
| 8 | CDK6 | 000534 | CDK6-V16G | ADQQYECG16AEIG | C | ADQQYECV16AEIG | C |
| | | | | EGAYGK | | EGAYGK | |
| | | | | (SEQ ID NO: 32) | | (SEQ ID NO: 33) | |
| 9 | FGFR1 | P11362 | FGFR1-E338K | NVSFEDAGK338 | | NVSFEDAGE338YTC | long (36 |
| | | | | (SEQ ID NO: 34) | | LAGNSIGLSHHSAWL | aa) |
| | | | | | | TVLEALEER | |
| | | | | | | (SEQ ID NO: 35) | |
| 10 | GATA3 | P23771 | GATA3-S390L | NSL390FNPAALSR | | NSS390FNPAALSR | |
| | | | | (SEQ ID NO: 36) | | (SEQ ID NO: 37) | |
| 11 | GATA3 | P23771 | GATA3-E383K | VHDSLK383 | short | VHDSLE383DFPK | |
| | | | | (SEQ ID NO: 38) | (6 aa) | (SEQ ID NO: 39) | |
| 12 | PTEN | P60484 | PTEN-D92N | VAQYPFEN92H | | VAQYPFED92H | |
| | | | | NPPQLELIK | | NPPQLEL1K | |
| | | | | (SEQ ID NO: 40) | | (SEQ ID NO: 41) | |
| 13 | PTEN | P60484 | PTEN-H123Y | PFCEDLDQWLS | 2C | PFCEDLDQWLS | 2C |
| | | | | EDDNHVAAI | | EDDNHVAAI | |
| | | | | Y123CK | | H123CK | |
| | | | | (SEQ ID NO: 42) | | (SEQ ID NO: 43) | |
| 14 | RBI | P06400 | RB1-F570C | IMESLAWLSDS | M, C | IMESLAWLSDS | M |
| | | | | PLC570DLIK | | PLF570DLIK | |
| | | | | (SEQ ID NO: 44) | | (SEQ ID NO: 45) | |
| 15 | RBI | P06400 | RB1-R876P | LP876FDIEGS | | | Short |
| | | | | DEADGSK | | | {2 aa) |
| | | | | (SEQ ID NO: 46) | | | |
| 16 | STKI1 | Q15831 | STKI1-F264V | LV264ENIGK | | LF264ENIGK | |
| | | | | (SEQ ID NO: 47) | | (SEQ ID NO: 48) | |
| 17 | ERBB2 | P04626 | ERBB2-R217H | CWGESSEDCQSL | 4C | CWGESSEDCQS | 2C |
| | | | | TH217TVC | | LTR217 (SEQ | |
| | | | | AGGCAR | | ID NO: 50) | |
| | | | | (SEQ ID NO: 49) | | | |
| 18 | ERBB2 | P04626 | ERBB2-R217C | CWGESSEDCQ | 5C | CWGESSEDCQS | 2C |
| | | | | SLTC217TVC | | LTR217 (SEQ | |
| | | | | AGGCAR | | ID NO: 50) | |
| | | | | (SEQ ID NO: 51) | | | |
| 19 | ERBB2 | P04626 | ERBB2-S310F | YTFGASCVTAC | Long | YTFGASCVTA | Long |
| | | | | PYNYLSTDV | (42 aa) | CPYNYLSTDV | (41 |
| | | | | GF310CTLVC | | GS310CTLVC | aa) |
| | | | | PLHNQEVTAE | | PLHNQEVTAED | |
| | | | | DGTQR | | GTQR | |
| | | | | (SEQ ID NO: 53) | | (SEQ ID NO: 54) | |
| 20 | ERBB2 | P04626 | ERBB2-S310Y | YTFGASCVTAC | Long | YTFGASCVTA | Long |
| | | | | PYNYLSTDV | (42 aa) | CPYNYLSTDV | (41 |
| | | | | GY310CTLVC | | GS310CTLVC | aa) |
| | | | | PLHNQEVTAE | | PLHNQEVTAED | |
| | | | | DGTQR | | GTQR | |
| | | | | (SEQ ID NO: 55) | | (SEQ ID NO: 54) | |
| 21 | ERBB2 | P04626 | ERBB2-R683Q | | | | Short |
| | | | | | | | (2 aa) |
| 22 | ERBB2 | P04626 | ERBB2-Q709L | LLQETELVE | M | LLQETELVEPL | M |
| | | | | PLTPSGAMP | | TPSGAMPN | |
| | | | | NL709AQMR | | Q709AQMR | |
| | | | | (SEQ ID NO: 56) | | (SEQ ID NO: 57) | |
| 23 | ERBB2 | P04626 | ERBB2-E717D | D717TELR | short (5 aa) | E717TELR | Short |
| | | | | (SEQ ID NO: 168) | | (SEQ ID NO: 168) | (4 aa) |
| 24 | ERBB2 | P04626 | ERBB2-L755S | VS755R | short (3 aa) | VL755R | short |
| | | | | | | | (3aa) |
| 25 | ERBB2 | P04626 | ERBB2-I767M | EM767LDEAY | M | EI767LDEAYV | M |
| | | | | VMAGVGSPY | | MAGVGSPYVS | |
| | | | | VSR | | R | |
| | | | | (SEQ ID NO: 58) | | (SEQ ID NO: 59) | |
| 26 | ERBB2 | P04626 | ERBB2-D769H | EILH769EAY | M | EILD769EAYVM | M |
| | | | | VMAGVGSPYV | | AGVGSPYVSR | |
| | | | | SR | | (SEQ ID NO: 59) | |
| | | | | (SEQ ID NO: 158) | | | |
| 27 | ERBB2 | P04626 | ERBB2-D769Y | EILY769EAYV | M | EILD769EAYVM | M |
| | | | | MAGVGSPYV | | AGVGSPYVS | |
| | | | | SR | | R | |
| | | | | (SEQ ID NO: 159) | | (SEQ ID NO: 59) | |
| 28 | ERBB2 | P04626 | ERBB2-V777L | EILDEAYVMAG | M | EILDEAYVMAG | M |
| | | | | L777GSPYVS | | V777GSPYVS | |
| | | | | R | | R | |
| | | | | (SEQ ID NO: 160) | | (SEQ ID NO: 59) | |
| 29 | ERBB2 | P04626 | ERBB2-L869R | R869 | short | L869LDIDETE | |
| | | | | | (1 aa) | YHADGGK | |
| | | | | | | (SEQ ID NO: 169) | |
| 30 | ESR1 | P03372 | ESR1-E380Q | VPGFVDLTLHD | Long | VPGFVDLTLHDQ | long (31 |
| | | | | QVHLLQ380 | (31 aa) | VHLLE380CAWL | aa) |
| | | | | CAWLEILMIG | | EILMIGLVWR | |
| | | | | LVWR (SEQ ID | | (SEQ ID | |
| | | | | NO: 60) | | NO: 61) | |
| 31 | ESR1 | P03372 | ESR1-S463P | SIILLNSGVYT | | SIILLNSGVYTF | |
| | | | | FLP463STLK | | LS463STLK | |
| | | | | (SEQ ID NO: 62) | | (SEQ ID NO: 63) | |
| 32 | ESR1 | P03372 | ESR1-I487M | ITDTLM487HL | M | ITDTLI487HL | M |
| | | | | MAK (SEQ ID | | MAK (SEQ ID | |
| | | | | NO: 64) | | NO: 65) | |
| 33 | ESR1 | P03372 | ESR1-L536P | NVVPP536YDL | M | NVVPL536YDLL | M |
| | | | | LLEMLDAHR | | LEMLDAHR | |
| | | | | (SEQ ID NO: 66) | | (SEQ ID NO: 67) | |
| 34 | ESR1 | P03372 | ESR1-L536R | NVVPR536 | short | NVVPL536YDLL | M |
| | | | | (SEQ ID NO: 68) | (5 aa) | LEMLDAHR | |
| | | | | | | (SEQ ID NO: 67) | |
| 35 | ESR1 | P03372 | ESR1-L536H | NVVPH536YDL | M | NVVPL536YDLL | M |
| | | | | LLEMLDAHR | | LEMLDAHR | |
| | | | | (SEQ ID NO: 69) | | (SEQ ID NO: 67) | |
| 36 | ESR1 | P03372 | ESR1-Y537S | NVVPLS537DL | M | NVVPLY537DLL | M |
| | | | | LLEMLDAHR | | LEMLDAHR | |
| | | | | (SEQ ID NO: 70) | | (SEQ ID NO: 67) | |
| 37 | ESR1 | P03372 | ESR1-Y537N | NVVPLN537DL | M | NVVPLY537DLL | M |
| | | | | LLEMLDAHR | | LEMLDAHR | |
| | | | | (SEQ ID NO: 71) | | (SEQ ID NO: 67) | |
| 38 | ESR1 | P03372 | ESR1-Y537C | NVVPLC537DLL | M | NVVPLY537DLL | M |
| | | | | LEMLDAHR | | LEMLDAHR | |
| | | | | (SEQ ID NO: 72) | | (SEQ ID NO: 67) | |
| 39 | ESR1 | P03372 | ESR1-Y537D | NVVPLD537DL | M | NVVPLY537DLLL | M |
| | | | | LLEMLDAHR | | EMLDAHR | |
| | | | | (SEQ ID NO: 73) | | (SEQ ID NO: 67) | |
| 40 | ESR1 | P03372 | ESR1-Y537G | NVVPLG537DL | M | NVVPLY537DLLL | M |
| | | | | LLEMLDAHR | | EMLDAHR | |
| | | | | (SEQ ID NO: 74) | | (SEQ ID NO: 67) | |
| 41 | ESR1 | P03372 | ESR1-Y537H | NVVPLH537DLL | M | NVVPLY537DLL | M |
| | | | | LEMLDAHR | | LEMLDAHR | |
| | | | | (SEQ ID NO: 75) | | (SEQ ID NO: 67) | |
| 42 | ESR1 | P03372 | ESR1-D538G | NVVPLYG538LL | M | NVVPLYD538LL | M |
| | | | | LEMLDAHR | | LEMLDAHR | |
| | | | | (SEQ ID NO: 76) | | (SEQ ID NO: 67) | |
| 43 | PIK3CA | P42336 | P1K3CA-R38H | ILVECLLPNGMI | 2C | ILVECLLPNGMIV | M, 2C |
| | | | | VTLECLH38 | | TLECLR38 | |
| | | | | EATLIT1K | | (SEQ ID NO: 171) | |
| | | | | (SEQ ID NO: 170) | | | |
| 44 | PIK3CA | P42336 | PIK3CA-E81K | EK81 | Short | EE81FFDETR | |
| | | | | | (2 aa) | (SEQ ID NO: 78) | |
| 45 | PIK3CA | P42336 | PIK3CA- | EA81FFDETR | | EE81FFDETR | |
| | | | E81A | (SEQ ID NO: 77) | | (SEQ ID NO: 78) | |
| 46 | PIK3CA | P42336 | PIK3CA- | Q88LCDLR | short | 88LCDLR | short (5 |
| | | | R88Q | (SEQ ID NO: 79) | (6 aa) | (SEQ ID NO: 80) | aa). C |
| 47 | PIK3CA | P42336 | PIK3CA- | LCDLQ93LFQ | C | LCDLR93 | short (5 |
| | | | R930 | PFLK (SEQ ID | | (SEQ ID NO: 80) | aa). C |
| | | | | NO: 81) | | | |
| 48 | PIK3CA | P42336 | PIK3CA- | VIEPVV106NR | | VIEPVG106NR | |
| | | | G106V | (SEQ ID NO: | | (SEQ ID NO: | |
| | | | | 82) | | 83) | |
| 49 | PIK3CA | P42336 | PIK3CA- | V1EPVGT107R | | VIEPVGN107R | |
| | | | N107T | (SEQ ID NO: | | (SEQ ID NO: | |
| | | | | 84) | | 83) | |
| 50 | PIK3CA | P42336 | PIK3CA- | VIEPVGNH108E | | VIEPVGNR108 | |
| | | | R108H | EK (SEQ ID | | (SEQ ID NO: | |
| | | | | NO: 85) | | 83) | |
| 51 | PIK3CA | P42336 | PIK3CA- | EIDII8FAIGMP | C | EIGI18FAIGMP | 2M.C |
| | | | G118D | VCEFDMVK | | VCEFDM VK | |
| | | | | (SEQ ID NO: 86) | | (SEQ ID NO: 87) | |
| 52 | PIK3CA | P42336 | PIK3CA- | ILCATYVK345 | c | ILCATYVN345VNIR | C |
| | | | N345K | (SEQ ID NO: | | (SEQ ID | |
| | | | | 88) | | NO: 89) | |
| 53 | PIK3CA | P42336 | PIK3CA- | TGIYHGGK365 | OK | TGIYHGGE365PL | C |
| | | | E365K | (SEQ ID NO: | | CDNVNTQR | |
| | | | | 90) | | (SEQ ID NO: 91) | |
| 54 | PIK3CA | P42336 | PIK3CA- | EEHR420 | short | EEHC420PLAWG | C |
| | | | C420R | (SEQ ID NO: 92) | (4 aa) | NINLFDYTD | |
| | | | | | | TLVSGK | |
| | | | | | | (SEQ ID NO: 93) | |
| 55 | PIK3CA | P42336 | PIK3CA- | MALNLWPVPHV451L | M | MALNLWPVPH | M |
| | | | G45IV | EDLLNPIGVTGSNP | | G451LEDLLN | |
| | | | | NK (SEQ ID NO: | | PIGVTGSNPNK | |
| | | | | 161) | | (SEQ ID NO: | |
| | | | | | | 95) | |
| 56 | PIK3CA | P42336 | PIK3CA- | MALNLWPVPHGL | M | MALNLWPVPHG | M |
| | | | E453K | K453 (SEQ | | LE453DLLN | |
| | | | | ID NO: 94) | | PIGVTGSNPNK | |
| | | | | | | (SEQ ID NO: | |
| | | | | | | 95) | |
| 57 | PIK3CA | P42336 | PIK3CA- | MALNLWPVPHGL | M | MALNLWPVPHG | M |
| | | | E453Q | Q453DLLN | | LE453DLLN | |
| | | | | PIGVTGSNPNK | | PIGVTGSNPNK | |
| | | | | (SEQ ID NO: | | (SEQ ID NO: | |
| | | | | 162) | | 95) | |
| 58 | PIK3CA | P42336 | PIK3CA- | MALNLWPVPHGL | M | MALNLWPVPHG | M |
| | | | E453G | G453DLLN | | LE453DLLN | |
| | | | | PIGVTGSNPNK | | PIGVTGSNPNK | |
| | | | | (SEQ ID NO: | | (SEQ ID NO: 95) | |
| | | | | 163) | | | |
| 59 | PIK3CA | P42336 | PIK3CA- | DR539 | short | DP539LSEITE | |
| | | | P539R | | (2 aa) | QEK (SEQ ID | |
| | | | | | | NO: 97) | |
| 60 | PIK3CA | P42336 | PIK3CA- | DPLSK542 | short | DPLSE542IT | |
| | | | E542K | (SEQ ID NO: 96) | (5 aa) | EQEK (SEQ ID | |
| | | | | | | NO: 97) | |
| 61 | PIK3CA | P42336 | P1K3CA-E542Q | DPLSQ5421TEQEK | | DPLSE542ITEQEK | |
| | | | | (SEQ ID NO: 98) | | (SEQ ID NO: 97) | |
| 62 | PIK3CA | P42336 | PIK3CA-E545K | DPLSEITK545 | | DPLSEITE545QEK | |
| | | | | (SEQ ID NO: 99) | | (SEQ ID NO: 97) | |
| 63 | P1K3CA | P42336 | PIK3CA-E545Q | DPLSEITQ545QEK | | DPLSEITE545QEK | |
| | | | | (SEQ ID NO: 100) | | (SEQ ID NO: 97) | |
| 64 | PIK3CA | P42336 | P1K3CA-E545G | DPLSEITG545QEK | | DPLSEITE545QEK | |
| | | | | (SEQ ID NO: 101) | | (SEQ ID NO: 97) | |
| 65 | PIK3CA | P42336 | PIK3CA- | DPLSEITER546 | | DPLSEITEQ546EK | |
| | | | 0546R | (SEQ ID NO: 102) | | (SEQ ID NO: 97) | |
| 66 | PIK3CA | P42336 | PIK3CA- | DPLSEITEK546 | | DPLSEITEQ546EK | |
| | | | Q546K | (SEQ ID NO: 103) | | (SEQ ID NO: 97) | |
| 67 | PIK3CA | P42336 | PIK3CA-E726K | DK726 | short | DE726TQK | short (5 |
| | | | | | (2 aa) | (SEQ ID NO: 104) | aa) |
| 68 | PIK3CA | P42336 | PIK3CA-G914E | SCAGYCVATFILGI | 2C | SCAGYCVATFILGI | 2C |
| | | | | E914DR | | G914DR | |
| | | | | (SEQ ID NO: 105) | | (SEQ ID NO: 106) | |
| 69 | PIK3CA | P42336 | PIK3CA- | QHANLFINLFSI1004 | 2M | QHANLFINLFSM1004 | 3M |
| | | | M10041 | MLGSGMPELQSFDD | | MLGSG | |
| | | | | IAYIR | | MPELQSFDDIAYIR | |
| | | | | (SEQ ID NO: 164) | | (SEQ ID NO: 172) | |
| 70 | PIK3CA | P42336 | PIK3CA- | S1025LALDK | short | T1025LALDK | short (6 |
| | | | T1025S | (SEQ ID NO: 107) | (6 aa) | (SEQ ID NO: 108) | aa) |
| 71 | PIK3CA | P42336 | PIK3 CA- | TLALN1029K | short | TLALD1029K | short (6 |
| | | | DI 029N | (SEQ ID NO: 109) | (6 aa) | (SEQ ID NO: 108) | aa) |
| 72 | PIK3CA | P42336 | PIK3CA- | TEQEALK1037 | OK | TEQEALE1037YFMK | M |
| | | | E1037K | (SEQ ID NO: 110) | | (SEQ ID NO: 111) | |
| 73 | PIK3CA | P42336 | PIK3CA- | QMK1044 | short | QMN1044DAHHGGWTTK | M |
| | | | N1044K | | (3 aa) | (SEQ ID NO: 113) | |
| 74 | PIK3CA | P42336 | PIK3CA- | QMNDAR1047 | short | QMNDAH1047HGGWTTK | M |
| | | | H1047R | (SEQ ID NO: 112) | (6 aa) | (SEQ ID NO: 113) | |
| 75 | PIK3CA | P42336 | PIK3CA- | QMNDAL1047HGGWTTK | M | QMNDAH1047HGGWTTK | M |
| | | | H1047L | (SEQ ID NO: 114) | | (SEQ ID NO: 113) | |
| 76 | PIK3CA | P42336 | PIK3CA- | QMNDAQ1047HGGWTTK | M | QMNDAH1047HGGWTTK | M |
| | | | H1047Q | (SEQ ID NO: 115) | | (SEQ ID NO: 113) | |
| 77 | PIK3CA | P42336 | PIK3CA- | QMNDAHHR1049 | M | QMNDAHHG1049GWTTK | M |
| | | | G1049R | (SEQ ID NO: 116) | | (SEQ ID NO: 113) | |
| 78 | TP53 | P04637 | TP53-Y126N | SVTCTN126SPALNK | C | SVTCTY126SPALNK | C |
| | | | | (SEQ ID NO: 117) | | (SEQ ID NO: 118) | |
| 79 | TP53 | P04637 | TP53-K132R | SVTCTYSPALNR132 | C | SVTCTYSPALNK132 | C |
| | | | | (SEQ ID NO: 119) | | (SEQ ID NO: 118) | |
| 80 | TP53 | P04637 | TP53-K132E | SVTCTYSPALNE132M | 2C, M | SVTCTYSPALNK132 | c |
| | | | | FCQLAK | | (SEQ ID NO: 118) | |
| | | | | (SEQ ID NO: 120) | | | |
| 81 | TP53 | P04637 | TP53-C135Y | MFY135QLAK | M | MFC135QLAK | c |
| | | | | (SEQ ID NO: 121) | | (SEQ ID NO: 122) | |
| 82 | TP53 | P04637 | TP53-V143M | TCPM143QLWVDSTP | C, M | TCPV143QLWVDSTPP | c |
| | | | | PPGTR | | PGTR | |
| | | | | (SEQ ID NO: 123) | | (SEQ ID NO: 124) | |
| 83 | TP53 | P04637 | TP53-P151S | TCPVQLWVDSTS151 | C | TCPVQLWVDSTP151P | c |
| | | | | PPGTR | | PGTR | |
| | | | | (SEQ ID NO: 173) | | (SEQ ID NO: 124) | |
| 84 | TP53 | P04637 | TP53-P151H | TCPVQLWVDSTH151 | C | TCPVQLWVDSTP151P | c |
| | | | | PPGTR | | PGTR | |
| | | | | (SEQ ID NO: 174) | | (SEQ ID NO: 124) | |
| 85 | TP53 | P04637 | TP53-P152L | TCPVQLWVDSTPL152 | C | TCPVQLWVDSTPP152 | c |
| | | | | PGTR | | PGTR | |
| | | | | (SEQ ID NO: 175) | | (SEQ ID NO: 124) | |
| 86 | TP53 | P04637 | TP53-T1551 | TCPVQLWVDSTPPPG | C | TCPVQLWVDSTPPPG | c |
| | | | | I155R | | T155R | |
| | | | | (SEQ ID NO: 176) | | (SEQ ID NO: 124) | |
| 87 | TP53 | P04637 | TP53-R156P | TCPVQLWVDSTPPPGT | C | TCPVQLWVDSTPPP | c |
| | | | | P156VR | | GTR156 | |
| | | | | (SEQ ID NO: 177) | | (SEQ ID NO: 124) | |
| 88 | TP53 | P04637 | TP53-V172F | QSQHMTEF172VR | M | QSQHMTEV172VR | M |
| | | | | (SEQ ID NO: 125) | | (SEQ ID NO: 126) | |
| 89 | TP53 | P04637 | TP53-V173M | QSQHMTEVM173R | M | QSQHMTEVV173R | M |
| | | | | (SEQ ID NO: 127) | | (SEQ ID NO: 126) | |
| 90 | TP53 | P04637 | TP53-R175H | H175CPHHER | C | R175 | short (1 |
| | | | | (SEQ ID NO: 178) | | | aa) |
| 91 | TP53 | P04637 | TP53-HI79R | CPHR179 | short | CPHH179ER | short (6 |
| | | | | (SEQ ID NO: 179) | (4 aa) | (SEQ ID NO: 180) | aa) |
| 92 | TP53 | P04637 | TP53-H179Y | CPHY179ER | short | CPHH179ER | short (6 |
| | | | | (SEQ ID NO: 181) | (5 aa) | (SEQ ID NO: 180) | aa) |
| 93 | TP53 | P04637 | TP53-H193L | CSDSDGLAPPQ | C | CSDSDGLAPPQ | C |
| | | | | L193LIR | | H193LIR | |
| | | | | (SEQ ID NO: 128) | | (SEQ ID NO: 129) | |
| 94 | TP53 | P04637 | TP53-H214R | R214 | short | H214SVWPYEP | long |
| | | | | | (1 aa) | PEVGSDCTT | (35 |
| | | | | | | IHYNYMCNS | aa), |
| | | | | | | SCMGGMNR | 3C |
| | | | | | | (SEQ ID NO: 182) | |
| 95 | TP53 | P04637 | TP53-P219S | HSVVVS219YEP | Ions | HSVVVP219YE | long |
| | | | | PEVGSDCTT | (35 aa) | PPEVGSDCTT | (35 |
| | | | | IHYNYMCNSSCM | | IHYNYMCNSSC | aa), |
| | | | | GGMNR | | MGGMNR | 3C |
| | | | | (SEQ ID NO: 183) | | (SEQ ID NO: 182) | |
| 96 | TP53 | P04637 | TP53-Y220C | HSVVVPC220EP | Ions | HSVVVPY220E | long |
| | | | | PEVGSDCTT | (35 aa) | PPEVGSDCTT | (35 |
| | | | | IHYNYMCNSSC | | IHYNYMCNSSC | aa), |
| | | | | MGGMNR | | MGGMNR | 3C |
| | | | | (SEQ ID NO: 184) | | (SEQ ID NO: 182) | |
| 97 | TP53 | P04637 | TP53-C238S | HSVVVPYEPPE | Ions | HSVVVPYEPPEV | long |
| | | | | VGSDCTTIH | (35 aa) | GSDCTTIHY | (35 |
| | | | | YNYMS238NSS | | NYMC238NSSC | aa), |
| | | | | CMGGMNR | | MGGMNR | 3C |
| | | | | (SEQ ID NO: 185) | | (SEQ ID NO: 182) | |
| 98 | TP53 | P04637 | TP53-C238F | HSVVVPYEPPE | Ions | HSVVVPYEPPEV | long |
| | | | | VGSDCTTIH | (35 aa) | GSDCTTIHY | (35 |
| | | | | YNYMF238NSS | | NYMC238NSS | aa), |
| | | | | CMGGMNR | | CMGGMNR | 3C |
| | | | | (SEQ ID NO: 186) | | (SEQ ID NO: 182) | |
| 99 | TP53 | P04637 | TP53-C238Y | HSVVVPYEPPE | Ions | HSVVVPYEPPE | long |
| | | | | VGSDCTTIH | (35 aa) | VGSDCTTIHY | (35 |
| | | | | YNYMY238NS | | NYMC238NSS | aa), |
| | | | | SCMGGMNR | | CMGGMNR | 3C |
| | | | | (SEQ ID NO: 187) | | (SEQ ID NO: 182) | |
| 1(X) | TP53 | P04637 | TP53-C242F | HSVVVPYEPPE | Ions | HSVVVPYEPPE | long |
| | | | | VGSDCTTIH | (35 aa) | VGSDCTTIHY | (35 |
| | | | | YNYMCNSSF242 | | NYMCNSSC242 | aa), |
| | | | | MGGMNR | | MGGMNR | 3C |
| | | | | (SEQ ID NO: 188) | | (SEQ ID NO: 182) | |
| 101 | TP53 | P04637 | TP53-G244D | HSVVVPYEPPE | Ions | HSVVVPYEPPE | long |
| | | | | VGSDCTTIH | (35 aa) | VGSDCTTIHY | (35 |
| | | | | YNYMCNSSCM | | NYMCNSSCM | aa), |
| | | | | D244GMNR | | G244GMNR | 3C |
| | | | | (SEQ ID NO: 189) | | (SEQ ID NO: 182) | |
| 102 | TP53 | P04637 | TP53-G245S | HSVVVPYEPP | Ions | HSVVVPYEPPE | long |
| | | | | EVGSDCTTIH | (35 aa) | VGSDCTTIHY | (35 |
| | | | | YNYMCNSSCM | | NYMCNSSCMGG | aa), |
| | | | | GS245MNR | | 245MNR | 3C |
| | | | | (SEQ ID NO: 190) | | (SEQ ID NO: 182) | |
| 103 | TP53 | P04637 | TP53-M246V | HSVVVPYEPPE | Ions | HSVVVPYEPP | long |
| | | | | VGSDCTTIH | (35 aa) | EVGSDCTTIHY | (35 |
| | | | | YNYMCNSSCM | | NYMCNSSCMG | aa), |
| | | | | GGV246NR | | GM246NR | 3C |
| | | | | (SEQ ID NO: 191) | | (SEQ ID NO: 182) | |
| 104 | TP53 | P04637 | TP53-R248W | HSVVVPYEPP | Ions | HSVVVPYEPP | long |
| | | | | EVGSDCTTIH | (35 aa) | EVGSDCTTIHY | (35 |
| | | | | YNYMCNSSCM | | NYMCNSSCMG | aa), |
| | | | | GGMNW248R | | GMNR248 | 3C |
| | | | | (SEQ ID NO: 192) | | (SEQ ID NO: 182) | |
| 105 | TP53 | P04637 | TP53-R248Q | HSVVVPYEPPE | long | HSVWPYEPPE | long (35 |
| | | | | VGSDCTTIH | (35 aa) | VGSDCTTIHY | aa). 3C |
| | | | | YNYMCNSSCMG | | NYMCNSSCM | |
| | | | | GMNQ248R | | GGMNR248 | |
| | | | | (SEQ ID NO: 193) | | (SEQ ID NO: 182) | |
| 106 | TP53 | P04637 | TP53-R249S | S249PILTIIT | | (R)249PILTII | indirect |
| | | | | LEDSSGNLLGR | | TLEDSSGNLLGR | (cleavage) |
| | | | | (SEQ ID NO: 130) | | (SEQ ID NO: 131) | |
| 107 | TP53 | P04637 | TP53-F270L | NSL270EVR | short | NSF270EVR | short (6 |
| | | | | (SEQ ID NO: 132) | (6 aa) | (SEQ ID NO: 133) | aa) |
| 108 | TP53 | P04637 | TP53-R273H | NSFEVH273V | 2C | NSFEVR273 | short (6 |
| | | | | CACPGR (SEQ | | (SEQ ID NO: 133) | aa) |
| | | | | ID NO: 134) | | | |
| 109 | TP53 | P04637 | TP53-R273C | NSFEVC273V | 3C | NSFEVR273 | short (6 |
| | | | | CACPGR (SEQ | | (SEQ ID NO: 133) | aa) |
| | | | | ID NO: 135) | | | |
| 110 | TP53 | P04637 | TP53-P278R | VCACR278 | short | VCACP278GR | 2C |
| | | | | (SEQ ID NO: 194) | (5 aa) | (SEQ ID NO: | |
| | | | | | | 137) | |
| 111 | TP53 | P04637 | TP53-R280G | VCACPGG280 | 2C | VCACPGR280 | 2C |
| | | | | DR (SEQ ID | | (SEQ ID NO: | |
| | | | | NO: 136) | | 137) | |
| 112 | TP53 | P04637 | TP53-R282G | DG282R | short | DR282 | short (2 |
| | | | | | (3 aa) | | aa) |
| 113 | TP53 | P04637 | TP53-E286V | TEV286ENLR | | TEE286ENLR | |
| | | | | (SEQ ID NO: | | (SEQ ID NO: | |
| | | | | 138) | | 139) | |

TABLE 6
1PANEL

| | | Therapy- | | | | | | | | | |
|---|---|---|---|---|---|---|---|---|---|---|---|
| TOP 50 | Included | relevant | | | | | | | | | |
| alteration | in Guardant | (ESR1, | | | | | | | | | |
| of TCGA | panel (for | HER2, | | | | | knownEf- | | | ClinicalSig- | |
| dataset | ctDNA) | PIK3CA) | Freq. | Percent | Cum. | oncogenic | fect | Summary | Level | nificance | Class |
| X | X | X | 32 | 0.47 | 11.11 | Oncogenic | Gain-of- | The | LEVEL_3A | Uncertain | single |
| | | | | | | | function | ERBB2 | | significance | nucleotide |
| | | | | | | | | V777L | | | variant |
| | | | | | | | | mutation is | | | |
| | | | | | | | | known to | | | |
| | | | | | | | | be | | | |
| | | | | | | | | oncogenic. | | | |
| X | X | X | 17 | 0.25 | 22.98 | Oncogenic | Gain-of- | The | LEVEL_3A | Likely | single |
| | | | | | | | function | ERBB2 | | pathogenic | nucleotide |
| | | | | | | | | L755S | | | variant |
| | | | | | | | | mutation is | | | |
| | | | | | | | | known to | | | |
| | | | | | | | | be | | | |
| | | | | | | | | oncogenic. | | | |
| | X | X | 16 | 0.24 | 23.71 | Oncogenic | Gain-of- | The | LEVEL_3A | Likely | single |
| | | | | | | | function | ERBB2 | | pathogenic | nucleotide |
| | | | | | | | | S310F | | | variant |
| | | | | | | | | mutation is | | | |
| | | | | | | | | known to | | | |
| | | | | | | | | be | | | |
| | | | | | | | | oncogenic. | | | |
| | X | X | 5 | 0.07 | 64.72 | Oncogenic | Gain-of- | The | LEVEL_3A | Likely | single |
| | | | | | | | function | ERBB2 | | pathogenic | nucleotide |
| | | | | | | | | S310Y | | | variant |
| | | | | | | | | mutation is | | | |
| | | | | | | | | known to | | | |
| | | | | | | | | be | | | |
| | | | | | | | | oncogenic. | | | |
| | | | 10 | 0.15 | 42.35 | Likely | Likely | The | LEVEL_3A | Uncertain | single |
| | | | | | | Oncogenic | Gain-of- | ERBB2 | | significance | nucleotide |
| | | | | | | | function | R683Q | | | variant |
| | | | | | | | | mutation is | | | |
| | | | | | | | | likely | | | |
| | | | | | | | | oncogenic. | | | |
| | X | | 7 | 0.1 | 55.25 | Oncogenic | Gain-of- | The | LEVEL_3A | Pathogenic | single |
| | | | | | | | function | ERBB2 | | | nucleotide |
| | | | | | | | | L869R | | | variant |
| | | | | | | | | mutation is | | | |
| | | | | | | | | known to | | | |
| | | | | | | | | be | | | |
| | | | | | | | | oncogenic. | | | |
| | | | 6 | 0.09 | 59.43 | Likely | Likely | The | LEVEL_3A | NA | NA |
| | | | | | | Oncogenic | Gain-of- | ERBB2 | | | |
| | | | | | | | function | R217H | | | |
| | | | | | | | | mutation | | | |
| | | | | | | | | has not | | | |
| | | | | | | | | been | | | |
| | | | | | | | | functionally | | | |
| | | | | | | | | or clinically | | | |
| | | | | | | | | validated. | | | |
| | | | | | | | | However, | | | |
| | | | | | | | | ERBB2 | | | |
| | | | | | | | | R217C is | | | |
| | | | | | | | | likely | | | |
| | | | | | | | | oncogenic, | | | |
| | | | | | | | | and | | | |
| | | | | | | | | therefore | | | |
| | | | | | | | | ERBB2 | | | |
| | | | | | | | | R217H is | | | |
| | | | | | | | | considered | | | |
| | | | | | | | | likely | | | |
| | | | | | | | | oncogenic. | | | |
| | | | 1 | 0.01 | 94.39 | Likely | Likely | The | LEVEL_3A | NA | NA |
| | | | | | | Oncogenic | Gain-of- | ERBB2 | | | |
| | | | | | | | function | R217C | | | |
| | | | | | | | | mutation is | | | |
| | | | | | | | | likely | | | |
| | | | | | | | | oncogenic. | | | |
| | | | 5 | 0.07 | 64.57 | Likely | Unknown | The | LEVEL_3A | NA | NA |
| | | | | | | Oncogenic | | ERBB2 | | | |
| | | | | | | | | E717D | | | |
| | | | | | | | | mutation | | | |
| | | | | | | | | has not | | | |
| | | | | | | | | been | | | |
| | | | | | | | | functionally | | | |
| | | | | | | | | or clinically | | | |
| | | | | | | | | validated. | | | |
| | | | | | | | | However, | | | |
| | | | | | | | | ERBB2 | | | |
| | | | | | | | | E717K is | | | |
| | | | | | | | | known to | | | |
| | | | | | | | | be | | | |
| | | | | | | | | oncogenic, | | | |
| | | | | | | | | and | | | |
| | | | | | | | | therefore | | | |
| | | | | | | | | ERBB2 | | | |
| | | | | | | | | E717D is | | | |
| | | | | | | | | considered | | | |
| | | | | | | | | likely | | | |
| | | | | | | | | oncogenic. | | | |
| | X | | 4 | 0.06 | 71.43 | Oncogenic | Gain-of- | The | LEVEL_3A | NA | NA |
| | | | | | | | function | ERBB2 | | | |
| | | | | | | | | I767M | | | |
| | | | | | | | | mutation is | | | |
| | | | | | | | | known to | | | |
| | | | | | | | | be | | | |
| | | | | | | | | oncogenic. | | | |
| | | | 4 | 0.06 | 71.49 | Oncogenic | Gain-of- | The | LEVEL_3A | NA | NA |
| | | | | | | | function | ERBB2 | | | |
| | | | | | | | | Q709L | | | |
| | | | | | | | | mutation is | | | |
| | | | | | | | | known to | | | |
| | | | | | | | | be | | | |
| | | | | | | | | oncogenic. | | | |
| | X | X | 2 | 0.03 | 84.75 | Oncogenic | Gain-of- | The | LEVEL_3A | Pathogenic/ | single |
| | | | | | | | function | ERBB2 | | Likely | nucleotide |
| | | | | | | | | D769H | | pathogenic | variant |
| | | | | | | | | mutation is | | | |
| | | | | | | | | known to | | | |
| | | | | | | | | be | | | |
| | | | | | | | | oncogenic. | | | |
| | X | X | 2 | 0.03 | 84.78 | Oncogenic | Gain-of- | The | LEVEL_3A | Pathogenic/ | single |
| | | | | | | | function | ERBB2 | | Likely | nucleotide |
| | | | | | | | | D769Y | | pathogenic | variant |
| | | | | | | | | mutation is | | | |
| | | | | | | | | known to | | | |
| | | | | | | | | be | | | |
| | | | | | | | | oncogenic. | | | |

TABLE 7
variants_breast_annotated

| Gene | Variant |
|---|---|
| PIK3CA | H1047R |
| ESR1 | D538G |
| PIK3CA | E542K |
| PIK3CA | E545K |
| TP53 | R175H |
| TP53 | R273H |
| ESR1 | Y537S |
| ESR1 | E380Q |
| PIK3CA | C420R |
| AKT1 | E17K |
| ERBB2 | V777L |
| TP53 | R248W |
| CDK6 | V16G |
| PIK3CA | H1047L |
| TP53 | R273C |
| GNAS | R201H |
| PIK3CA | E726K |
| TP53 | K132R |
| TP53 | R248Q |
| TP53 | H214R |
| ESR1 | L536P |
| ESR1 | Y537N |
| CCNE1 | W330L |
| KIT | V654A |
| TP53 | H193L |
| STK11 | F264V |
| ERBB2 | L755S |
| TP53 | E286V |
| ERBB2 | S310F |
| PTEN | T277K |
| TP53 | C238S |
| PTEN | R130G |
| TP53 | P219S |
| AR | V716M |
| TP53 | C238F |
| TP53 | H179R |
| TP53 | P278R |
| TP53 | V143M |
| BRCA2 | Y2660C |
| GATA3 | S391L |
| IDH1 | R132H |
| PIK3CA | G118D |
| TP53 | C238Y |
| TP53 | C242F |
| TP53 | P151S |
| TP53 | Y126N |
| RB1 | F570C |
| ATM | R337H |
| ESR1 | I487M |
| TP53 | C135Y |
| TP53 | G245S |
| TP53 | R280G |
| ERBB2 | R683Q |
| FGFR2 | N549K |
| TP53 | Y220C |
| ESR1 | L536R |
| PIK3CA | N345K |
| TP53 | T155I |
| ESR1 | Y537C |
| PIK3CA | E542Q |
| PIK3CA | P539R |
| TP53 | V173M |
| ERBB2 | L869R |
| ESR1 | L536H |
| NRAS | G12C |
| PIK3CA | D1029N |
| RB1 | R876P |
| TP53 | K132E |
| BRAF | V600E |
| ERBB2 | R217H |
| FGFR1 | E338K |
| PIK3CA | G1049R |
| BRCA2 | R2842H |
| ATM | R3008C |
| CCNE1 | Y296H |
| ERBB2 | E717D |
| ERBB2 | S310Y |
| KRAS | G12D |
| KRAS | G13D |
| KRAS | K117N |
| KRAS | V14I |
| MAP2K1 | R47Q |
| MAP2K2 | N126T |
| CDKN2A | P81L |
| GATA3 | E384K |
| PIK3CA | Q546R |
| PIK3CA | R93Q |
| PTEN | C136R |
| SMAD4 | E330K |
| TP53 | F270L |
| TP53 | G244D |
| TP53 | H179Y |
| TP53 | M246V |
| TP53 | P151H |
| TP53 | P152L |
| TP53 | R156P |
| TP53 | R249S |
| TP53 | R282G |
| TP53 | V172F |
| ATM | R3008H |
| ERBB2 | I767M |
| ERBB2 | Q709L |
| HRAS | G13V |
| KRAS | G12V |
| PIK3CA | R88Q |
| CDKN2A | A36V |
| EGFR | T790M |
| ESR1 | S463P |
| KIT | S480Y |
| KRAS | G12C |
| KRAS | G12R |
| NRAS | G12D |
| PIK3CA | E453K |
| PIK3CA | E453Q |
| PIK3CA | E545Q |
| PIK3CA | Q546K |
| PTEN | R130Q |
| ATM | R337C |
| BRAF | L485F |
| BRAF | N581S |
| BRCA2 | R2842C |
| ERBB2 | D769H |
| ERBB2 | D769Y |
| HRAS | Q61L |
| MTOR | Y1463F |
| PIK3CA | E81K |
| PIK3CA | H1047Q |
| PIK3CA | N1044K |
| PIK3CA | T1025S |
| PTEN | D92N |
| PTEN | H123Y |
| ARAF | P216R |
| ATM | R2832H |
| BRAF | D594G |
| BRAF | D594N |
| BRAF | V600M |
| BRCA1 | R1699W |
| BRCA2 | G2748D |
| ERBB2 | R103Q |
| ERBB2 | R217C |
| ESR1 | Y537D |
| ESR1 | Y537G |
| ESR1 | Y537H |
| HRAS | F28S |
| HRAS | G12D |
| HRAS | G12S |
| HRAS | Q61H |
| IDH1 | R132S |
| KIT | E562K |
| KIT | V560A |
| KRAS | A146T |
| KRAS | G12A |
| PIK3CA | E1037K |
| PIK3CA | E365K |
| PIK3CA | E453G |
| PIK3CA | E545G |
| KRAS | Q61H |
| PIK3CA | E81A |
| PIK3CA | G106V |
| PIK3CA | G451V |
| PIK3CA | G914E |
| PIK3CA | M1004I |
| PIK3CA | N107T |
| PIK3CA | R108H |
| PIK3CA | R38H |
| RET | C634Y |
| TP53 | R196* |
| TP53 | R213* |
| PTEN | Q245* |
| CDH1 | Q351* |
| TP53 | Q38* |
| ARID1A | S1791* |
| SMAD4 | Q245* |
| TP53 | Y163* |
| NF1 | E785* |
| BRCA1 | Q1525* |
| ARID1A | Q625* |
| ERBB2 | A775_G776insVA |
| NF1 | Q535* |
| PTEN | Q171* |
| APC | S393* |
| PTEN | Q214* |
| TP53 | E224* |
| TP53 | Q167* |
| ATM | L2946* |
| SMAD4 | W99* |
| TP53 | R342* |
| BRACA2 | S617* |
| PTEN | E235* |
| TP53 | E204* |
| ATM | R3047* |
| CDKN2A | R80* |
| NF1 | Q1341* |
| PTEN | E91* |
| TP53 | Q192* |
| BRCA1 | Q1240* |
| BRCA2 | R2520* |
| CDK12 | E887* |
| NF1 | Q1399* |
| NF1 | S1030* |
| PTEN | Q17* |
| BRCA2 | E2193* |
| BRCA2 | S3356* |
| ATM | Q2637* |
| BRCA1 | E1221* |
| BRCA2 | Q2456* |
| NF1 | R1241* |
| PTEN | R41* |
| BRCA1 | S1363* |
| BRCA1 | S770* |
| BRCA2 | F1192* |
| BRCA2 | W563* |
| PIK3CA | G460del |
| NOTCH1 | S341R |
| ATM | K618E |
| PDGFRA | R764C |
| RAF1 | V537I |
| AKT1 | R174H |
| NF1 | L1876R |
| APC | D1512N |
| AR | E323K |
| MYC | S264R |
| NF1 | I1911V |
| PIK3CA | L540V |
| RHOA | E47K |
| TERT | Q73P |
| ARID1A | P198L |
| NF1 | H1143Y |
| NF1 | V921L |
| PIK3CA | I84IV |
| RBI | T5P |
| TSC1 | G1016S |
| ALK | A1440T |
| ALK | S1487L |
| ATM | N2879S |
| FGFR2 | D304N |
| MAP2K2 | N113H |
| MET | I1115V |
| PDGFRA | P60R |
| SMAD4 | R496C |
| TERT | C7G |
| AR | E32K |
| AR | Q641K |
| KIT | R281K |
| MET | I865T |
| MET | R547Q |
| PDGFRA | E927K |
| PDGFRA | R522H |
| ALK | L1555P |
| ARAF | E195A |
| ARID1A | W2091R |
| EGFR | K327N |
| KIT | G872V |
| KIT | L160F |
| MTOR | R553H |
| ALK | R1373K |
| APC | E1544Q |
| AR | Q62L |
| CCND1 | L165V |
| EGFR | V674I |
| FGFR2 | P413P |
| NF1 | E715A |
| NF1 | S1420L |
| PDGFRA | V859M |
| TERT | D685N |
| TERT | R669Q |
| TP53 | C277Y |
| TP53 | H179D |
| APC | R213Q |
| ARID1A | P1280S |
| AR | Q488* |
| ATM | K3018N |
| ATM | N2697I |
| ATM | P2699L |
| ATM | Y332H |
| BRAF | G327V |
| BRAF | L190V |
| CCND2 | P281L |
| CDH1 | L116L |
| EGFR | G917R |
| ERBB2 | D582N |
| ERBB2 | E286K |
| ERBB2 | T105I |
| FBXW7 | P4P |
| FGFR2 | A704S |
| FGFR3 | L723L |
| MAPK3 | A378T |
| MYC | F38L |
| NF1 | A1424V |
| PDGFRA | A491D |
| PIK3CA | D454N |
| RB1 | T12P |
| RHOA | G17E |
| APC | G309E |
| APC | APC_N2098S |
| APC | APC_S92Y |
| ARID1A | ARID1A_E2224Q |
| AR | AR_S244L |
| ATM | ATM_I352F |
| BRCA1 | BRCA1_R1076K |
| BRCA2 | BRCA2_L3277V |
| EGFR | EGFR_A647T |
| EGFR | EGFR_K860N |
| ERBB2 | ERBB2_L720L |
| HNF1A | HNF1A_E274K |
| HNF1A | HNF1A_R272H |
| MAP2K2 | MAP2K2_G132D |
| MET | MET_R1148Q |
| MET | MET_S1353F |
| NF1 | NF1_A1224A |
| NF1 | NF1_S1567L |
| PDGFRA | PDGFRA_I1076I |
| RAF1 | RAF1_V88V |
| RB1 | RB1_R775S |
| RB1 | RB1_S249L |
| TP53 | TP53_V272M |
| TP53 | TP53_Y163D |
| ALK | ALK_I1383T |
| AR | AR_P378S |
| CCND1 | CCND1_V293M |
| CDKN2A | CDKN2_AA102V |
| EGFR | EGFR_E548Q |
| ERBB2 | ERBB2_F534L |
| ERBB2 | ERBB2_G727A |
| ERBB2 | ERBB2_R340Q |
| FBXW7 | FBXW7_R479* |
| FGFR1 | FGFR1_D60N |
| GNAS | GNAS_R201S |
| MET | MET_A1363T |
| MET | MET_E168D |
| MET | MET_I883T |
| NF1 | NF1_C383S |
| NF1 | NF1_Y2698H |
| NOTCH1 | NOTCH1_S2533F |
| PDGFRA | PDGFRA_E1068* |
| PDGFRA | PDGFRA_R500Q |
| PIK3CA | PIK3CA_R88* |
| TP53M160 | TP53_M160_A161del |
| TP53 | TP53_M237I |
| TP53 | TP53_S241A |
| ARAF | ARAF_D491H |
| ARID1A | ARID1A_M618I |
| ARID1A | ARID1A_Q2219H |
| ARID1A | ARID1A_S1134A |
| ATM | ATM_N3003D |
| BRCA1 | BRCA1_S1796L |
| BRCA2 | BRCA2_Exon 11 Deletion |
| BRCA2 | BRCA2_M965I |
| CCND1 | CCND1_V77V |
| CDK12 | CDK12_E765K |
| DDR2 | DDR2_N617S |
| ERBB2 | ERBB2_E507K |
| ERBB2 | ERBB2_S413L |
| FGFR2 | FGFR2_S453L |
| GNAS | GNAS_R201C |
| KIT | KIT_L970V |
| MET | MET_M39I |
| NF1 | NF1_L2290L |
| NF1 | NF1_Q28Q |
| NF1 | NF1_V903M |
| NOTCH1 | NOTCH1_E2515K |
| NOTCH1 | NOTCH1_S225L |
| NOTCH1 | NOTCH1_V220V |
| PDGFRA | PDGFRA_K910K |
| RB1 | RB1_D918N |
| SMO | SMO_I530I |
| SMO | SMO_R547H |
| TP53 | TP53_E336K |
| TP53 | TP53_M243V |
| TP53 | TP53_R158H |
| APC | APC_G1116D |
| APC | APC_S1588L |
| APC | APC_S2129L |
| ARID1A | ARID1A_A1626A |
| ARID1A | ARID1A_D2086A |
| ARID1A | ARID1A_E1531K |
| ARID1A | ARID1A_P1326Q |
| ARID1A | ARID1A_Q515* |
| ARID1A | ARID1A_Q766P |
| ARIDIA | ARID1A_R693R |
| AR | AR_A141T |
| AR | AR_D296H |
| AR | AR_E622K |
| AR | AR_G462D |
| AR | AR_G744E |
| BRAF | BRAF_I543I |
| BRAF | BRAF_R252* |
| BRCA1 | BRCA1_D1269E |
| BRCA2 | BRCA2_D3170N |
| BRCA2 | BRCA2_E1577Q |
| BRCA2 | BRCA2_E2650K |
| BRCA2 | BRCA2_P3189L |
| CCND1 | CCND1_R291W |
| CCND2 | CCND2_L21R |
| CCND2 | CCND2_R22Q |
| CDH1 | CDH1_N390N |
| CDK6 | CDK6_R214H |
| CDKN2A | CDKN2A_L30L |
| CDKN2B | CDKN2B_G113G |
| EGFR | EGFR_A1195V |
| EGFR | EGFR_A822T |
| EGFR | EGFR_R832H |
| ERBB2 | ERBB2_R536W |
| ESR1 | ESR1_V422del |
| FGFR1 | FGFR1_D69A |
| FGFR1 | FGFR1_E562E |
| FGFR1 | FGFR1_S136L |
| FGFR1 | FGFR1_S789C |
| FGFR1 | FGFR1_V394V |
| FGFR2-KIAA1598 | FGFR2-KIAA1598_Fusion |
| FGFR2 | FGFR2_R330W |
| FGFR3 | FGFR3_S783L |
| GNAS | GNAS_I207I |
| IDH2 | IDH2_K166K |
| KIT | KIT_R830* |
| MAPK3 | MAPK3_F36F |
| MET | MET_D1286N |
| MET | MET_E868* |
| MET | MET_P44P |
| NF1 | NF1_E337K |
| NF1 | NF1_R1526R |
| NF1 | NF1_S636F |
| NOTCH1 | NOTCH1_S2121R |
| NOTCH1 | NOTCH1_S223R |
| PTEN | PTEN_D236N |
| RB1_c.1957 | RB1_c.1957_1960 + 27del |
| RET | RET_E805Q |
| ROS1 | ROS1_K1904T |
| VHL | VHL_V62G |
| APC | APC_D1636N |
| APC | APC_H753Y |
| APC | APC_I2181T |
| APC | APC_I2541I |
| APC | APC_P2261P |
| APC | APCS104L |
| APC | APCS384R |
| APC | APCT175T |
| ARID1A | ARJD1AP1456A |
| ARID1A | ARIDlA_P854fs |
| ARID1A | ARID1AQ1342* |
| ARID1A | ARID1AQ878* |
| ARID1A | ARID1AS304L |
| AR | ARA22D |
| AR | ARC785Y |
| AR | ARS215L |
| ATM | ATM_G2777D |
| ATM | ATMR1610T |
| ATM | ATMY2019C |
| BRCA1 | BRCA1_R1204K |
| BRCA2 | BRCA2_D2566H |
| BRCA2D935 | BRCA2_D935_A938delinsYMT |
| BRCA2 | BRCA2_P606P |
| CCND1 | CCND1_V193V |
| CCND2 | CCND2_I287I |
| CCND2 | CCND2_P94P |
| CCNE1 | CCNE1_R145Q |
| CDH1 | CDH1_R63* |
| CDK12 | CDK12_I925fs |
| CDK12 | CDK12_Q1307fs |
| EGFR | EGFR_H805Q |
| EGFR | EGFR_L907L |
| EGFR | EGFR_P644P |
| EGFR | EGFR_Q1159L |
| EGFR | EGFR_R671H |
| EGFR | EGFR_Y113Y |
| ERBB2 | ERBB2_A598D |
| ERBB2 | ERBB2_C224F |
| ERBB2 | ERBB2_S609C |
| ESR1 | ESR1_P535R |
| ESR1_Q565 | ESRl_Q565_H567del |
| FGFR1 | FGFR1_N546K |
| GATA3 | GATA3_T441fs |
| HNF1A | HNF1A_L185L |
| IDH1 | IDH1_I129I |
| KIT | KIT_D419N |
| KIT | KIT_G658G |
| MET_c.2888-20 | MET_c.2888-20_2888-4del |
| MYC | MYC_P57S |
| NF1 | NF1_G1219R |
| NF1 | NF1_K2652fs |
| NF1 | NF1_P1432Q |
| NF1 | NF1_R125C |
| NF1 | NF1_V2511V |
| NF1_W1831 | NF1_W1831_E1832del |
| NOTCH1 | NOTCH1_N1682S |
| NOTCH1 | NOTCH1_S2537C |
| NOTCH1 | NOTCH1_T2483M |
| PDGFRA | PDGFRA_Splice Site SNV |
| PTEN | PTEN_D368D |
| RB1 | RB1_M708I |
| RB1 | RB1_Q689L |
| ROS1 | ROS1_A1711S |
| ROS1 | ROS1_S1891I |
| SMAD4 | SMAD4_E377Q |
| SMAD4 | SMAD4_S154* |
| SMAD4 | SMAD4_T521I |
| TERT | TERT_S656S |
| TERT | TERT_W36G |
| TP53_A276 | TP53_A276_P278del |
| TP53 | TP53_C135* |
| TP53 | TP53_C135F |
| TP53 | TP53_C176Y |
| TP53 | TP53_E258* |
| TP53 | TP53_E271Q |
| TP53 | TP53_E285K |
| TP53 | TP53_G356A |
| TP53 | TP53_N235fs |
| TP53 | TP53_P152fs |
| TP53 | TP53_P278H |
| TP53 | TP53_R110P |
| TP53 | TP53_R267W |
| TP53 | TP53_R273G |
| TP53 | TP53_R282W |
| TP53 | TP53_R306* |
| TP53 | TP53_R337C |
| TP53 | TP53_S106fs |
| TP53 | TP53_V197L |
| TP53 | TP53_Y205F |
| TP53 | TP53_c.1101-2del |
| ALK | ALK_R1214C |
| APC | APC_G277S |
| APC | APC_Q1447* |
| APC | APC_Q901E |
| APC | APC_R2237* |
| ARID1A | ARID1A_D1258N |
| ARID1A | ARID1A_D204A |
| ARID1A | ARID1A_E1297* |
| ARID1A | ARID1A_E1778K |
| ARID1A | ARID1A_H860H |
| ARID1A | ARID1A_I2192I |
| ARID1A | ARID1A_L2056I |
| ARID1A | ARID1A_P225P |
| ARID1A | ARID1A_P469S |
| ARID1A | ARID1A_Q1334Q |
| ARID1A | ARID1A_Q566* |
| ARID1A | ARID1A_R693Q |
| ARID1A | ARID1A_S261* |
| ARID1A | ARID1A_V1717A |
| ATM | ATM_G2695R |
| ATM | ATM_H2538Q |
| ATM | ATM_L176Q |
| BRAF | BRAF_D179E |
| BRAF | BRAF_E204D |
| BRAF | BRAF_G518G |
| BRAF | BRAF_I666M |
| BRCA1 | BRCA1_A1175A |
| BRCA1 | BRCA1_E349D |
| BRCA1 | BRCA1_S426L |
| BRCA1 | BRCA1_V1234fs |
| BRCA2 | BRCA2_R2896H |
| BRCA2 | BRCA2_S1538C |
| BRCA2 | BRCA2_S3192S |
| CCNE1 | CCNE1_G342G |
| CCNE1 | CCNE1_L108L |
| CCNE1 | CCNE1_L140M |
| CCNE1 | CCNE1_P396S |
| CDH1 | CDH1_T79fs |
| CDH1 | CDH1_V365V |
| EGFR | EGFR_A613T |
| EGFR | EGFR_D1014Y |
| EGFR | EGFR_E282K |
| EGFR | EGFR_K189M |
| EGFR | EGFR_L1198fs |
| EGFR | EGFR_Q105Q |
| ERBB2 | ERBB2_A705A |
| ERBB2 | ERBB2_D769D |
| ERBB2 | ERBB2_E238K |
| ERBB2 | ERBB2_I628M |
| ERBB2 | ERBB2_L181L |
| ERBB2 | ERBB2_P593R |
| ERBB2 | ERBB2_P617A |
| ESR1 | ESR1_F461I |
| ESR1 | ESR1_G442R |
| ESR1 | ESR1_T347T |
| FBXW7 | FBXW7_L648P |
| FGFR1 | FGFR1_E496K |
| FGFR2 | FGFR2_W4L |
| GATA3 | GATA3_A319A |
| GATA3 | GATA3_R399fs |
| GATA3 | GATA3_S427fs |
| GATA3 | GATA3_S438fs |
| GATA3 | GATA3_Y345S |
| HNF1A | HNF1A_T196T |
| HNF1A | HNF1A_V259I |
| KIT | KIT_N99D |
| KIT | KIT_R135C |
| KIT | KIT_R224K |
| MAPK1 | MAPK1_F329L |
| MAPK3 | MAPK3_I319I |
| MET | MET_D1101H |
| MET | MET_E436K |
| MET | MET_G672S |
| MET | MET_K599N |
| MET | MET_M1013I |
| MET | MET_P664L |
| MYC | MYC_L114R |
| MYC | MYC_L214F |
| MYC | MYC_L84V |
| MYC | MYC_P246Q |
| NF1 | NF1_E1344L |
| NF1 | NF1_L1978L |
| NF1 | NF1_L2380L |
| NF1 | NF1_R2814C |
| NF1 | NF1_Y80S |
| NF1_c.7063-2 | NF1_c.7063-2_7065del |
| NOTCH1 | NOTCH1_A2452V |
| NOTCH1 | NOTCH1_E1636K |
| NOTCH1 | NOTCH1_L2149L |
| NOTCH1 | NOTCH1_P143L |
| NOTCH1 | NOTCH1_P1566P |
| NTRK1 | NTRK1_R347C |
| NTRK1 | NTRK1_V647L |
| PDGFRA | PDGFRA_H974Y |
| PDGFRA | PDGFRA_I119V |
| PDGFRA | PDGFRA_V484M |
| PIK3CA | PIK3CA_D1017H |
| PIK3CA | PIK3CA_I1058L |
| RAF1 | RAF1_N635S |
| RAF1 | RAF1_R627W |
| RET | RET_F644F |
| RET | RET_R678R |
| ROS1 | ROS1_I1844I |
| SMAD4 | SMAD4_P246R |
| STK11 | STK11_H174D |
| STK11 | STK11_W308C |
| TERT | TERT_P64Q |
| TP53 | TP53_A276G |
| TP53 | TP53_C176S |
| TP53 | TP53_D228fs |
| TP53 | TP53_G266E |
| TP53 | TP53_H179Q |
| TP53 | TP53_H193R |
| TP53 | TP53_I195T |
| TP53 | TP53_I255S |
| TP53 | TP53_L194F |
| TP53 | TP53_N239S |
| TP53 | TP53_P177L |
| TP53 | TP53_P295fs |
| TP53 | TP53_P318fs |
| TP53 | TP53_Q167fs |
| TP53 | TP53_V272E |
| TP53 | TP53_W91* |
| TP53 | TP53_W91fs |
| TP53 | TP53_Y163N |
| TSC1 | TSC1_H1161D |
| TSC1 | TSC1_R1033T |
| AKT1 | AKT1_F237F |
| AKT1 | AKT1_K268K |
| ALK | ALK_E1605K |
| ALK | ALK_S1538S |
| APC | APC_A1358A |
| APC | APC_D2059N |
| APC | APC_E225K |
| APC | APC_E2655K |
| APC | APC_E847fs |
| APC | APC_K39N |
| APC | APC_L1657V |
| APC | APC_P1999P |
| APC | APC_Q8Q |
| APC | APC_S2575F |
| APC | APC_S982S |
| APC | APC_T2382I |
| ARID1A | ARID1A_A164A |
| ARID1A_A344 | ARID1A_A344_A348del |
| ARID1A | ARID1A_E1019* |
| ARID1A | ARID1A_G191fs |
| ARID1A | ARID1A_L69R |
| ARID1A | ARID1A_P1325P |
| ARID1A | ARID1A_P1560L |
| ARID1A | ARID1A_Q1066* |
| ARID1A | ARID1A_Q1415* |
| ARID1A | ARID1A_Q171Q |
| ARID1A | ARID1A_Q766fs |
| ARID1A | ARID1A_R1202G |
| ARID1A | ARID1A_S1937T |
| AR | AR_A201S |
| AR | AR_A420A |
| AR | AR_A587A |
| AR | AR_I2I |
| AR | AR_L194L |
| AR | AR_L713F |
| AR | AR_M1? |
| AR | AR_P383S |
| AR | AR_Q488E |
| AR | AR_R586R |
| AR | AR_S158* |
| AR | AR_S53G |
| AR | AR_T440K |
| AR | AR_W4* |
| ATM | ATM_D2721N |
| ATM | ATM_D2959N |
| ATM | ATM_E2429G |
| ATM | ATM_G2891D |
| ATM | ATM_I1035M |
| ATM | ATM_I1093V |
| ATM | ATM_K2318N |
| ATM | ATM_K797K |
| ATM | ATM_L2447V |
| ATM | ATM_L3017L |
| ATM | ATM_R2034P |
| ATM | ATM_R2526T |
| ATM | ATM_R720H |
| ATM | ATM_S3027G |
| ATM | ATM_V2441G |
| ATM | ATM_V2716I |
| ATM | ATM_Y2954D |
| BRAF | BRAF_G219A |
| BRAF | BRAF_R603Q |
| BRAF | BRAF_S136L |
| BRAF | BRAF_Splice Site SNV |
| BRCA1 | BRCA1_E1060K |
| BRCA1 | BRCA1_E1221K |
| BRCA1 | BRCA1_H279D |
| BRCA1 | BRCA1_K701K |
| BRCA1 | BRCA1_Q1396Q |
| BRCA1 | BRCA1_R1028C |
| BRCA1 | BRCA1_V1632V |
| BRCA2 | BRCA2_C1893Y |
| BRCA2 | BRCA2_E1555K |
| BRCA2 | BRCA2_E2239Q |
| BRCA2 | BRCA2_E3256V |
| BRCA2 | BRCA2_G379R |
| BRCA2 | BRCA2_N2374S |
| BRCA2 | BRCA2_Q699R |
| BRCA2 | BRCA2_S538R |
| BRCA2 | BRCA2_T2097A |
| CCND2 | CCND2_E2E |
| CCND2 | CCND2_L243L |
| CCND2 | CCND2_V91V |
| CCNE1 | CCNE1_A178A |
| CDH1 | CDH1_V114V |
| CDH1 | CDH1_Y68* |
| CDK12 | CDK12_E1345K |
| CDK12 | CDK12_E431Q |
| CDK12 | CDK12_I836M |
| CDK12 | CDK12_P1030S |
| CDK12 | CDK12_R93L |
| CDK12 | CDK12_T1071T |
| CDK12 | CDK12_V1297M |
| DDR2 | DDR2_L623V |
| DDR2 | DDR2_S667C |
| EGFR | EGFR_D770N |
| EGFR | EGFR_E513K |
| EGFR | EGFR_E711K |
| EGFR | EGFR_I646I |
| EGFR | EGFR_K80N |
| EGFR | EGFR_N552K |
| EGFR | EGFR_P564T |
| EGFR | EGFR_P644L |
| EGFR | EGFR_Q1143Q |
| EGFR | EGFR_R427H |
| EGFR | EGFR_S116fs |
| EGFR | EGFR_S186S |
| EGFR | EGFR_S895C |
| EGFR | EGFR_V1097I |
| ERBB2 | ERBB2_E1195K |
| ERBB2 | ERBB2_E207D |
| ERBB2 | ERBB2_E580K |
| ERBB2 | ERBB2_E619K |
| ERBB2 | ERBB2_E766Q |
| ERBB2 | ERBB2_G246S |
| ERBB2 | ERBB2_L458L |
| ERBB2 | ERBB2_L85L |
| ERBB2 | ERBB2_L96L |
| ERBB2 | ERBB2_R1146W |
| ERBB2 | ERBB2_R143Q |
| ERBB2 | ERBB2_R689T |
| ERBB2 | ERBB2_S457L |
| ERBB2 | ERBB2_S463S |
| ERBB2 | ERBB2_S963F |
| ERBB2 | ERBB2_Y1222H |
| ESR1 | ESR1_D545D |
| ESR1 | ESR1_K362N |
| ESR1 | ESR1_K520K |
| ESR1 | ESR1_R394C |
| FGFR1 | FGFR1_L269L |
| FGFR1 | FGFR1_S518L |
| FGFR1 | FGFR1_T111T |
| FGFR1 | FGFR1_V740V |
| FGFR2-CCDC6 | FGFR2-CCDC6_Fusion |
| FGFR2 | FGFR2_A171V |
| FGFR2 | FGFR2_A181A |
| FGFR2 | FGFR2_E116Q |
| FGFR2 | FGFR2_G272E |
| FGFR2 | FGFR2_H254Y |
| FGFR2 | FGFR2_L716L |
| FGFR2 | FGFR2_P443P |
| FGFR2 | FGFR2_T320T |
| FGFR3-TACC3 | FGFR3-TACC3_Fusion |
| FGFR3 | FGFR3_S408C |
| FGFR3 | FGFR3_S804S |
| GATA3 | GATA3_M357I |
| GATA3 | GATA3_P436fs |
| HNF1A | HNF1A_R229Q |
| HNF1A | HNF1A_R271W |
| HRAS | HRAS_R123R |
| IDH1 | IDH1_E84Q |
| IDH1 | IDH1_G131S |
| IDH1 | IDH1_G136E |
| IDH2 | IDH2_I153M |
| KIT | KIT_A621T |
| KIT | KIT_I172I |
| KIT | KIT_I235fs |
| KIT | KIT_I563fs |
| KIT | KIT_L148F |
| KIT | KIT_S729C |
| KIT | KIT_S729Y |
| KIT | KIT_V603fs |
| MAPK1 | MAPK1_D44N |
| MAPK3 | MAPK3_L154L |
| MAPK3 | MAPK3_S170fs |
| MAPK3 | MAPK3_V68M |
| MET | MET_F346F |
| MET | MET_G921E |
| MET | MET_I166T |
| MET | MET_K508K |
| MET | MET_Q926* |
| MET | MET_R412H |
| MET | MET_R417* |
| MET | MET_W911* |
| MPL | MPL_L513L |
| MTOR | MTOR_I975I |
| MTOR | MTOR_R960* |
| MTOR | MTOR_S678F |
| MTOR | MTOR_T714S |
| MYC | MYC_R331W |
| NF1 | NF1_D2482N |
| NF1 | NF1_Exon 10 Deletion |
| NF1 | NF1_Exon 3 Deletion |
| NF1 | NF1_L651L |
| NF1 | NF1_N1503S |
| NF1 | NF1_S137S |
| NF1 | NF1_S1786L |
| NFE2L2 | NFE2L2_Q87* |
| NOTCH1 | NOTCH1_A1634A |
| NOTCH1 | NOTCH1_D1560H |
| NOTCH1 | NOTCH1_E2103K |
| NOTCH1 | NOTCH1_L2434L |
| NOTCH1 | NOTCH1_P2128L |
| NOTCH1 | NOTCH1_P226L |
| NOTCH1 | NOTCH1_R2272C |
| NOTCH1 | NOTCH1_S2183F |
| NOTCH1 | NOTCH1_S2357R |
| NTRK1 | NTRK1_E388K |
| PDGFRA | PDGFRA_E298E |
| PDGFRA | PDGFRA_F678I |
| PDGFRA | PDGFRA_G898D |
| PDGFRA | PDGFRA_R981H |
| PDGFRA | PDGFRA_Y993Y |
| PIK3CA_E109 | PIK3CA_E109_L113delinsI |
| PIK3CA | PIK3CA_E291Q |
| PIK3CA | PIK3CA_I1058M |
| PIK3CA | PIK3CA_K723K |
| PIK3CA | PIK3CA_M1040I |
| PIK3CA | PIK3CA_R818C |
| PIK3CA | PIK3CA_S1003P |
| PIK3CA | PIK3CA_S405F |
| PTEN | PTEN_E242K |
| PTEN | PTEN_E352Q |
| PTEN | PTEN_F154F |
| PTEN | PTEN_P246P |
| PTEN | PTEN_P30A |
| PTPN11 | PTPN11_D94N |
| RAF1 | RAF1_A280V |
| RAF1 | RAF1_G544G |
| RAF1 | RAF1_P261R |
| RAF1 | RAF1_R73Q |
| RAF1 | RAF1_S52C |
| RAF1 | RAF1_V180V |
| RB1_E413 | RB1_E413_I422del |
| RB1 | RB1_L477V |
| RB1 | RB1_M708K |
| RB1 | RB1_S534fs |
| RET | RET_H594P |
| RET | RET_R721Q |
| RHOA | RHOA_R5W |
| RIT1 | RIT1_R106* |
| ROS1 | ROS1_A1921T |
| ROS1 | ROS1_I1716N |
| SMAD4 | SMAD4_Q366* |
| SMAD4 | SMAD4_R361C |
| SMAD4 | SMAD4_R361H |
| SMAD4 | SMAD4_S242* |
| STK11 | STK11_W239C |
| TERT | TERT_G32W |
| TP53 | TP53_A159V |
| TP53 | TP53_A364A |
| TP53 | TP53_C242S |
| TP53 | TP53_D259V |
| TP53 | TP53_D259Y |
| TP53_E258 | TP53_E258_S260delinsA |
| TP53 | TP53_E286K |
| TP53 | TP53_E336* |
| TP53 | TP53_Exon 4 Deletion |
| TP53 | TP53_Exon 5 Insertion |
| TP53 | TP53_F113fs |
| TP53 | TP53_G154S |
| TP53 | TP53_G245C |
| TP53 | TP53_G266R |
| TP53 | TP53_G360V |
| TP53 | TP53_I195S |
| TP53 | TP53_K132Q |
| TP53 | TP53_L111R |
| TP53 | TP53_L111fs |
| TP53 | TP53_L265del |
| TP53 | TP53_M246I |
| TP53 | TP53_M246T |
| TP53 | TP53_P177R |
| TP53 | TP53_P190S |
| TP53 | TP53_Q331* |
| TP53 | TP53_R158G |
| TP53 | TP53_R280I |
| TP53 | TP53_R280K |
| TP53 | TP53_R280S |
| TP53 | TP53_S215R |
| TP53 | TP53_S46fs |
| TP53 | TP53_V143fs |
| TP53 | TP53_V73V |
| TP53 | TP53_Y107D |
| TP53 | TP53_Y163C |
| TP53 | TP53_Y205D |
| TP53 | TP53_Y234C |
| TP53_c.783-4 | TP53_c.783-4_792del |
| TP53 | TP53_c.920-2del |
| TSC1 | TSC1_E1101K |
| VHL | VHL_H125H |
| VHL | VHL_I109I |
| AKT1 | AKT1_P369P |
| AKT1 | AKT1_Q79K |
| AKT1 | AKT1_W80R |
| AKT1 | AKT1_Y176* |
| ALK | ALK_A1300fs |
| ALK | ALK_C1008R |
| ALK | ALK_E1065K |
| ALK | ALK_E1077Q |
| ALK | ALK_E1558Q |
| ALK | ALK_K1003K |
| ALK | ALK_R1120W |
| ALK | ALK_S11061 |
| ALK | ALK_Y1359H |
| APC | APC_A630fs |
| APC | APC_D1794H |
| APC | APC_D2729Y |
| APC | APC_E1020Q |
| APC | APC_E136K |
| APC | APC_E1726K |
| APC | APC_E2589K |
| APC | APC_E418K |
| APC | APC_E771K |
| APC | APC_G1702R |
| APC | APC_G817fs |
| APC | APC_I1779M |
| APC | APC_K2695N |
| APC | APC_L589L |
| APC | APC_M337I |
| APC | APC_Q1444Q |
| APC | APC_Q203fs |
| APC | APC_Q445H |
| APC | APC_R2319T |
| APC | APC_R2434T |
| APC | APC_R2454T |
| APC | APC_R2543fs |
| APC | APC_S1144N |
| APC | APC_S2307L |
| APC | APC_S2307S |
| APC | APC_S2531C |
| APC | APC_S2768G |
| APC | APC_Splice Site SNV |
| APC | APC_T1074T |
| APC | APC_V754V |
| APC | APC_W2658* |
| ARAF | ARAF_R209H |
| ARAF | ARAF_R211H |
| ARID1A | ARID1A_D1810N |
| ARID1A | ARID1A_E1297K |
| ARID1A | ARID1A_E1802K |
| ARID1A | ARID1A_G889fs |
| ARID1A | ARID1A_H1581Q |
| ARID1A | ARID1A_H1871Y |
| ARID1A | ARID1A_L1405V |
| ARID1A | ARID1A_L1676L |
| ARID1A | ARID1A_N1502S |
| ARID1A | ARID1A_N2109S |
| ARID1A | ARID1A_P517S |
| ARID1A | ARID1A_Q1363P |
| ARID1A | ARIDlA_Q1519fs |
| ARID1A | ARID1A_Q2100E |
| ARID1A | ARID1A_Q268* |
| ARID1A | ARID1A_Q439L |
| ARID1A | ARID1A_Q501* |
| ARID1A | ARID1A_Q510* |
| ARID1A | ARID1A_Q557* |
| ARID1A | ARID1A_Q581* |
| ARID1A | ARID1A_Q840H |
| ARID1A | ARID1A_R1046K |
| ARID1A | ARID1A_R1202W |
| ARID1A | ARID1A_R1461* |
| ARID1A | ARID1A_R1461Q |
| ARID1A | ARID1A_R1463C |
| ARID1A | ARID1A_R1721Q |
| ARID1A | ARID1A_R2236C |
| ARID1A | ARID1A_S1248* |
| ARID1A | ARID1A_S1544S |
| ARID1A | ARID1A_S1675Y |
| ARID1A | ARID1A_S2002F |
| ARID1A | ARID1A_S2262L |
| ARID1A | ARID1A_S334L |
| ARID1A | ARID1A_S506F |
| ARID1A | ARID1A_S519Y |
| ARID1A | ARIDlA_Splice Site SNV |
| ARID1A | ARID1A_T2060S |
| ARID1A | ARID1A_V1464V |
| ARID1A | ARID1A_Y1101C |
| AR | AR_A236D |
| AR | AR_A358A |
| AR | AR_A417A |
| AR | AR_A52S |
| AR | AR_E204Q |
| AR | AR_E666K |
| AR | AR_E679D |
| AR | AR_F857L |
| AR | AR_G38G |
| AR | AR_G423V |
| AR | AR_G744R |
| AR | AR_K778R |
| AR | AR_K913T |
| AR | AR_N235N |
| AR | AR_P218S |
| AR | AR_R20Q |
| AR | AR_R407C |
| AR | AR_R407H |
| AR | AR_S165S |
| AR | AR_S186R |
| AR | AR_S885* |
| AR | AR_S909C |
| ATM | ATM_A2524T |
| ATM | ATM_D2016H |
| ATM | ATM_E2154Q |
| ATM | ATM_E2444G |
| ATM | ATM_F505F |
| ATM | ATM_H2872R |
| ATM | ATM_K147E |
| ATM | ATM_K2440E |
| ATM | ATM_K2717Q |
| ATM | ATM_L1125M |
| ATM | ATM_L2338L |
| ATM | ATM_L2946V |
| ATM | ATM_L3048L |
| ATM | ATM_P597S |
| ATM | ATM_Q2397R |
| ATM | ATM_Q2522H |
| ATM | ATM_R2034G |
| ATM | ATM_R2453H |
| ATM | ATM_R2973T |
| ATM | ATM_R493C |
| ATM | ATM_T1953I |
| ATM | ATM_T2773N |
| ATM | ATM_T909P |
| ATM | ATM_V2951F |
| ATM | ATM_V2951I |
| ATM | ATM_W2845L |
| BRAF | BRAF_L185L |
| BRAF | BRAF_S123C |
| BRAF | BRAF_S337* |
| BRAF | BRAF_S614F |
| BRCA1 | BRCA1_D1778G |
| BRCA1 | BRCA1_E1011K |
| BRCA1 | BRCA1_E1440Q |
| BRCA1 | BRCA1_E1829K |
| BRCA1 | BRCA1_G160G |
| BRCA1 | BRCA1_K947Q |
| BRCA1 | BRCA1_L1128V |
| BRCA1 | BRCA1_L1600F |
| BRCA1 | BRCA1_P1099T |
| BRCA1 | BRCA1_Q202H |
| BRCA1 | BRCA1_R1470K |
| BRCA1 | BRCA1_R1670K |
| BRCA1 | BRCA1_S282L |
| BRCA1 | BRCA1_S770L |
| BRCA2 | BRCA2_A1204T |
| BRCA2 | BRCA2_C2605S |
| BRCA2 | BRCA2_E1581D |
| BRCA2 | BRCA2_E2193V |
| BRCA2 | BRCA2_G1194D |
| BRCA2 | BRCA2_G2593R |
| BRCA2 | BRCA2_G267A |
| BRCA2 | BRCA2_H1905Y |
| BRCA2 | BRCA2_L1234V |
| BRCA2_M2192 | BRCA2_M2192_E2193del |
| BRCA2 | BRCA2_Q3206Q |
| BRCA2 | BRCA2_R1131T |
| BRCA2 | BRCA2_R2268K |
| BRCA2 | BRCA2_S1560N |
| BRCA2 | BRCA2_S1817C |
| BRCA2 | BRCA2_S2378L |
| BRCA2_V1392 | BRCA2_V1392_K1394del |
| BRCA2 | BRCA2_Y42fs |
| CCDC6-RET | CCDC6-RET_Fusion |
| CCND1 | CCND1_E256Q |
| CCND1 | CCND1_E51K |
| CCND1 | CCND1_L217L |
| CCND1 | CCND1_L32L |
| CCND1 | CCND1_Q264H |
| CCND1 | CCND1_R260fs |
| CCND1 | CCND1_S41L |
| CCNE1 | CCNE1_A214A |
| CCNE1 | CCNE1_A410V |
| CCNE1 | CCNE1_A47T |
| CCNE1 | CCNE1_D55N |
| CDH1 | CDH1_H123N |
| CDH1 | CDH1_R74* |
| CDH1 | CDH1_Splice Site SNV |
| CDH1 | CDH1_V391fs |
| CDH1 | CDH1_V412fs |
| CDK12 | CDK12_G479A |
| CDK12 | CDK12_G927fs |
| CDK12L823 | CDK12_L823_E825del |
| CDK12 | CDK12_N1081N |
| CDK12 | CDK12_R1484T |
| CDK12 | CDK12_S601S |
| CDK4 | CDK4_G42E |
| CDK4 | CDK4_L147V |
| CDK4 | CDK4_L213F |
| CDK6 | CDK6_L105F |
| CDK6 | CDK6_L42L |
| CDK6 | CDK6_P250P |
| CDKN2A | CDKN2A_A118V |
| CDKN2A | CDKN2A_G111A |
| CDKN2A | CDKN2A_H83Y |
| CDKN2A | CDKN2A_P18L |
| CDKN2A | CDKN2A_Q50fs |
| CDKN2A | CDKN2A_R87fs |
| CDKN2A | CDKN2A_S12L |
| CDKN2A | CDKN2A_S152L |
| CDKN2A | CDKN2A_W110* |
| CTNNB1 | CTNNB1_E55Q |
| DDR2 | DDR2_K720T |
| DDR2 | DDR2_L687V |
| DDR2 | DDR2_Q664Q |
| DDR2 | DDR2_Q791K |
| DDR2 | DDR2_R752H |
| EGFR | EGFR_A1201A |
| EGFR | EGFR_A864V |
| EGFR | EGFR_A92A |
| EGFR | EGFR_C231C |
| EGFR | EGFR_D1009D |
| EGFR | EGFR_D230N |
| EGFR | EGFR_D247N |
| EGFR | EGFR_E317K |
| EGFR | EGFR_E634K |
| EGFR | EGFR_E749K |
| EGFR | EGFR_E758* |
| EGFR | EGFR_H850R |
| EGFR | EGFR_K823R |
| EGFR | EGFR_L423L |
| EGFR | EGFR_L49V |
| EGFR | EGFR_L679L |
| EGFR | EGFR_M176I |
| EGFR | EGFR_M567I |
| EGFR | EGFR_N338N |
| EGFR | EGFR_P518P |
| EGFR | EGFR_P848P |
| EGFR | EGFR_Q40P |
| EGFR | EGFR_R149R |
| EGFR | EGFR_R255Q |
| EGFR | EGFR_R309Q |
| EGFR | EGFR_R958R |
| EGFR | EGFR_R962C |
| EGFR | EGFR_S1081S |
| EGFR | EGFR_W410* |
| EGFR | EGFR_W817S |
| ERBB2 | ERBB2_C540C |
| ERBB2 | ERBB2_F173F |
| ERBB2 | ERBB2_F258F |
| ERBB2 | ERBB2_G1056G |
| ERBB2 | ERBB2_G778A |
| ERBB2_G778 | ERBB2_G778_P780dup |
| ERBB2 | ERBB2_H174Y |
| ERBB2 | ERBB2_H267fs |
| ERBB2 | ERBB2_M955I |
| ERBB2 | ERBB2_N111N |
| ERBB2_P1116 | ERBB2_P1116_D1125del |
| ERBB2 | ERBB2_P197L |
| ERBB2 | ERBB2_Q527L |
| ERBB2 | ERBB2_Q828Q |
| ERBB2 | ERBB2_R1111W |
| ERBB2 | ERBB2_R351L |
| ERBB2 | ERBB2_V153V |
| ESR1 | ESR1_E419E |
| ESR1 | ESR1_L489L |
| ESR1 | ESR1_L539R |
| ESR1 | ESR1_L540P |
| ESR1 | ESR1_M528V |
| ESR1 | ESR1_Q314* |
| ESR1 | ESR1_R394R |
| ESR1 | ESR1_V392I |
| ESR1 | ESR1_V595V |
| ESR1 | ESR1_Y537Y |
| ESR1_Y537 | ESRl_Y537_D538insN |
| FBXW7 | FBXW7_C533S |
| FBXW7 | FBXW7_D643N |
| FBXW7 | FBXW7_S476C |
| FBXW7 | FBXW7_S641* |
| FBXW7 | FBXW7_T530T |
| FGFR1 | FGFR1_A354A |
| FGFR1 | FGFR1_A74A |
| FGFR1 | FGFR1_D110N |
| FGFR1 | FGFR1_D407N |
| FGFR1 | FGFR1_D527N |
| FGFR1 | FGFR1_K514K |
| FGFR1 | FGFR1_M731R |
| FGFR1 | FGFR1_P466P |
| FGFR1 | FGFR1_R250W |
| FGFR1 | FGFR1_R54C |
| FGFR1 | FGFR1_S104G |
| FGFR2 | FGFR2_C701S |
| FGFR2 | FGFR2_E467K |
| FGFR2 | FGFR2_G583R |
| FGFR2 | FGFR2_K74K |
| FGFR2 | FGFR2_L104L |
| FGFR2 | FGFR2_L10L |
| FGFR2 | FGFR2_L528F |
| FGFR2 | FGFR2_P775H |
| FGFR2_R111 | FGFR2_R111_T112delinsS |
| FGFR2 | FGFR2_R190W |
| FGFR2 | FGFR2_S252L |
| FGFR2 | FGFR2_T454M |
| FGFR3 | FGFR3_I698I |
| FGFR3 | FGFR3_Q263H |
| FGFR3 | FGFR3_S269S |
| GATA3 | GATA3_A396A |
| GATA3 | GATA3_K388fs |
| GATA3 | GATA3_M357fs |
| GATA3 | GATA3_P422fs |
| GATA3 | GATA3_R330K |
| GATA3 | GATA3_R367* |
| GATA3 | GATA3_S408fs |
| GATA3 | GATA3_T323fs |
| GATA3 | GATA3_T364fs |
| GATA3 | GATA3_V440fs |
| GNA11 | GNA11_E212K |
| GNAS | GNAS_Q227H |
| GNAS | GNAS_Q227L |
| GNAS | GNAS_T204A |
| HNF1A | HNF1A_P300P |
| HNF1A | HNF1A_S249* |
| IDH1 | IDH1_C114G |
| IDH1 | IDH1_F86L |
| IDH1 | IDH1_I130I |
| IDH1 | IDH1_I130V |
| JAK2 | JAK2_V617F |
| JAK3 | JAK3_A573V |
| KIT | KIT_D140D |
| KIT | KIT_D975N |
| KIT | KIT_L890R |
| KIT | KIT_M618V |
| KIT | KIT_R5G |
| KIT | KIT_S785S |
| KRAS | KRAS_Q25* |
| KRAS | KRAS_Q61* |
| KRAS | KRAS_R41S |
| MAP2K1 | MAP2K1_M143I |
| MAP2K2 | MAP2K2_H104H |
| MAP2K2 | MAP2K2_L122L |
| MAP2K2 | MAP2K2_V64F |
| MAPK1 | MAPK1_A307T |
| MAPK1 | MAPK1_E220Q |
| MAPK1 | MAPK1_H80P |
| MAPK1 | MAPK1_L234V |
| MAPK3 | MAPK3_E18Q |
| MAPK3 | MAPK3_E339A |
| MAPK3 | MAPK3_E351* |
| MAPK3 | MAPK3_I273I |
| MET | MET_A48T |
| MET | MET_D174H |
| MET | MET_D543E |
| MET | MET_E221K |
| MET | MET_E419K |
| MET | MET_E75E |
| MET | MET_E863Q |
| MET | MET_E999K |
| MET | MET_L180L |
| MET | MET_L614F |
| MET | MET_Q1029L |
| MET | MET_R1184Q |
| MET | MET_R1227S |
| MET | MET_S135S |
| MET | MET_T230T |
| MET | MET_V383V |
| MET | MET_V603D |
| MET | MET_V919I |
| MLH1 | MLH1_D376N |
| MLH1 | MLH1_L404V |
| MTOR | MTOR_C674S |
| MTOR | MTOR_D785G |
| MTOR | MTOR_E706Q |
| MTOR | MTOR_L660L |
| MTOR | MTOR_P189P |
| MTOR | MTOR_Q829Q |
| MTOR | MTOR_R112W |
| MTOR | MTOR_R910R |
| MTOR | MTOR_R957R |
| MTOR | MTOR_V420V |
| MYC | MYC_L442F |
| MYC | MYC_N45N |
| MYC | MYC_P58L |
| MYC | MYC_R314G |
| MYC | MYC_S344G |
| MYC | MYC_S79R |
| NF1 | NF1_C1711G |
| NF1 | NF1_C1930R |
| NF1 | NF1_D1644G |
| NF1 | NF1_E2578Q |
| NF1 | NF1_E725K |
| NF1 | NF1_L2023L |
| NF1 | NF1_N1451S |
| NF1 | NF1_P1638P |
| NF1 | NF1_Q369* |
| NF1 | NF1_R135fs |
| NF1 | NF1_R2179H |
| NF1 | NF1_R2452H |
| NF1 | NF1_R2594H |
| NF1 | NF1_S1329L |
| NF1 | NF1_S1754fs |
| NF1 | NF1_S495fs |
| NF1 | NF1_S883L |
| NF1 | NF1_Splice Site SNV |
| NF1 | NF1_V174fs |
| NF1 | NF1_V2799I |
| NFE2L2 | NFE2L2_E79K |
| NFE2L2 | NFE2L2_E79Q |
| NOTCH1 | NOTCH1_A1562A |
| NOTCH1 | NOTCH1_A2265fs |
| NOTCH1 | NOTCH1_A2463A |
| NOTCH1 | NOTCH1_D1609Y |
| NOTCH1 | NOTCH1_E1555G |
| NOTCH1 | NOTCH1_E198K |
| NOTCH1 | NOTCH1_E2103A |
| NOTCH1 | NOTCH1_P2445S |
| NOTCH1 | NOTCH1_P2525P |
| NOTCH1 | NOTCH1_S2073I |
| NOTCH1 | NOTCH1_V1599V |
| NOTCH1 | NOTCH1_Y219Y |
| NRAS | NRAS_R164C |
| NTRK1 | NTRK1_I572I |
| NTRK1 | NTRK1_M296K |
| NTRK1 | NTRK1_R654C |
| NTRK1 | NTRK1_S603C |
| NTRK1 | NTRK1_V630M |
| NTRK3 | NTRK3_G608G |
| NTRK3 | NTRK3_I695T |
| NTRK3 | NTRK3_P612H |
| NTRK3 | NTRK3_R582Q |
| PDGFRA | PDGFRA_C835C |
| PDGFRA | PDGFRA_D444Y |
| PDGFRA | PDGFRA_I269N |
| PDGFRA | PDGFRA_I647T |
| PDGFRA | PDGFRA_L825H |
| PDGFRA | PDGFRA_P441L |
| PDGFRA | PDGFRA_R979C |
| PDGFRA | PDGFRA_R981C |
| PDGFRA | PDGFRA_S145F |
| PDGFRA | PDGFRA_T99T |
| PDGFRA | PDGFRA_V129A |
| PDGFRA | PDGFRA_Y872* |
| PIK3CA | PIK3CA_E722K |
| PIK3CA | PIK3CA_G359R |
| PIK3CA | PIK3CA_G460D |
| PIK3CA | PIK3CA_I459V |
| PIK3CA | PIK3CA_L1067F |
| PIK3CA | PIK3CA_L287L |
| PIK3CA | PIK3CA_L586F |
| PIK3CA | PIK3CA_N426S |
| PIK3CA | PIK3CA_Q75E |
| PTEN | PTEN_D24V |
| PTEND24 | PTEN_D24_L25del |
| PTEN | PTEN_D92H |
| PTEN | PTEN_E43fs |
| PTEN | PTEN_F243V |
| PTEN | PTEN_G129E |
| PTEN | PTEN_G165R |
| PTEN | PTEN_H118Y |
| PTEN_I253 | PTEN_I253_V255del |
| PTEN | PTEN_I306L |
| PTEN | PTEN_K128T |
| PTEN | PTEN_L318F |
| PTEN | PTEN_M1? |
| PTEN | PTEN_N31D |
| PTEN | PTEN_Q110* |
| PTEN | PTEN_Q171E |
| PTEN | PTEN_R159K |
| PTEN | PTEN_S113fs |
| PTEN | PTEN_S229* |
| PTEN | PTEN_Y176* |
| PTEN | PTEN_Y176delins**CIIIH |
| PTEN | PTEN_Y178* |
| PTPN11 | PTPN11_A72T |
| PTPN11 | PTPN11_E76K |
| RAF1 | RAF1_H369Y |
| RAF1 | RAF1_K572K |
| RAF1 | RAF1_Q520* |
| RAF1 | RAF1_S567Y |
| RAF1 | RAF1_T543M |
| RB1 | RB1_C706F |
| RB1 | RB1_E551* |
| RB1 | RB1_E629* |
| RB1 | RB1_E677* |
| RB1 | RB1_F351fs |
| RB1 | RB1_I532I |
| RB1 | RB1_L797* |
| RB1 | RB1_M208V |
| RB1 | RB1_N522fs |
| RB1 | RB1_N541fs |
| RB1 | RB1_P23L |
| RB1 | RB1_P3P |
| RB1 | RB1_R254T |
| RB1 | RB1_R255* |
| RB1 | RB1_R656W |
| RB1 | RB1_R661W |
| RB1 | RB1_R787* |
| RB1 | RB1_R787R |
| RB1 | RB1_S163I |
| RB1 | RB1_S567* |
| RB1 | RB1_S618fs |
| RB1 | RB1_S807* |
| RB1 | RB1_S882L |
| RB1 | RB1_V654M |
| RB1_c.2212-1 | RB1_c.2212-1_2212del |
| RET | RET_E818E |
| RET | RET_Q781Q |
| RET | RET_V573V |
| RHOA | RHOA_G17V |
| RHOA | RHOA_I4F |
| RIT1 | RIT1_M90I |
| RIT1 | RIT1_S101C |
| ROS1 | ROS1_C1922F |
| ROS1 | ROS1_D2108Y |
| ROS1 | ROS1_G1915G |
| ROS1 | ROS1_P1938S |
| ROS1 | ROS1_T2045M |
| SMAD4 | SMAD4_A208V |
| SMAD4 | SMAD4_C363Y |
| SMAD4 | SMAD4_C523fs |
| SMAD4 | SMAD4_D351H |
| SMAD4 | SMAD4_D355G |
| SMAD4 | SMAD4_D493N |
| SMAD4 | SMAD4_E205A |
| SMAD4 | SMAD4_E330Q |
| SMAD4 | SMAD4_E337K |
| SMAD4 | SMAD4_E374D |
| SMAD4 | SMAD4_G247E |
| SMAD4 | SMAD4_I94M |
| SMAD4 | SMAD4_Q116* |
| SMAD4 | SMAD4_Q224* |
| SMAD4 | SMAD4_Q256* |
| SMAD4 | SMAD4_R445* |
| SMAD4 | SMAD4_S191S |
| SMAD4 | SMAD4_S485* |
| SMO | SMO_C314* |
| SMO | SMO_W331* |
| STK11 | STK11_L102P |
| STK11 | STK11_L290I |
| STK11 | STK11_P221L |
| STK11 | STK11_R39C |
| STK11 | STK11_R415G |
| STK11 | STK11_R42L |
| STK11 | STK11_S283C |
| STK11 | STK11_T13M |
| STK11T189 | STK11_T189_G196del |
| STK11_c.717 | STK11_c.717_734 + 8del |
| TERT | TERT_A651A |
| TERT | TERT_Promoter Indel |
| TERT | TERT_V39V |
| TP53 | TP53_C176G |
| TP53 | TP53_C242Y |
| TP53 | TP53_C275F |
| TP53 | TP53_C277fs |
| TP53 | TP53_D148H |
| TP53 | TP53_D184fs |
| TP53 | TP53_D208fs |
| TP53 | TP53_D281Y |
| TP53 | TP53_E198fs |
| TP53 | TP53_E258K |
| TP53 | TP53_E271K |
| TP53 | TP53_E271fs |
| TP53 | TP53_E285L |
| TP53 | TP53_E294* |
| TP53 | TP53_E339K |
| TP53 | TP53_E343* |
| TP53 | TP53_E51* |
| TP53 | TP53_Exon 5 Deletion |
| TP53 | TP53_Exon 7 Insertion |
| TP53 | TP53_F113del |
| TP53 | TP53_F134L |
| TP53 | TP53_F341C |
| TP53_G117 | TP53_G117_V122del |
| TP53 | TP53_G244C |
| TP53 | TP53_G245D |
| TP53 | TP53_G245fs |
| TP53 | TP53_H233fs |
| TP53 | TP53_I255N |
| TP53 | TP53_I50fs |
| TP53 | TP53_K101* |
| TP53 | TP53_K120R |
| TP53 | TP53_L130H |
| TP53 | TP53_L137Q |
| TP53 | TP53_L194R |
| TP53 | TP53_L252P |
| TP53 | TP53_L257P |
| TP53 | TP53_L45fs |
| TP53 | TP53_M246K |
| TP53 | TP53_N239fs |
| TP53 | TP53_P12P |
| TP53 | TP53_P151A |
| TP53 | TP53_Q104* |
| TP53 | TP53_Q317* |
| TP53 | TP53_R181P |
| TP53 | TP53_R248fs |
| TP53 | TP53_R280T |
| TP53 | TP53_R342G |
| TP53 | TP53_S116C |
| TP53 | TP53_S127T |
| TP53 | TP53_S149fs |
| TP53 | TP53_S215G |
| TP53 | TP53_S269fs |
| TP53 | TP53_S96fs |
| TP53 | TP53_T329fs |
| TP53 | TP53_V147fs |
| TP53 | TP53_V173L |
| TP53 | TP53_V197M |
| TP53_W146 | TP53_W146_T150delinsS |
| TP53 | TP53_Y163H |
| TP53 | TP53_Y205fs |
| TP53 | TP53_Y234del |
| TP53 | TP53_Y236C |
| TP53 | TP53_Y236N |
| TP53 | TP53_c.783-2del |
| TP53_c.916 | TP53_c.916_919 + 18del |
| TSC1 | TSC1_E1044fs |
| VHL | VHL_H115Y |
| NA | NA |
| Total | Total |

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for performing proteomic analysis on a sample, the method comprising treating the sample with a non-ionic surfactant, performing mass spectrometry on the treated sample, and detecting proteins in the treated sample.

2. The method of claim 1, wherein the non-ionic surfactant is an alkyl glucoside.

3. The method of claim 1, wherein the non-ionic surfactant is an alkyl diglucoside.

4. The method of claim 1, wherein the non-ionic surfactant is an alkyl maltoside.

5. The method of claim 1, wherein the non-ionic surfactant is octyl-maltoside, decyl-maltoside, dodecyl-maltoside, or tetradecyl-maltoside.

6. The method of claim 1, wherein the non-ionic surfactant is n-dodecyl-β-D-maltoside (DDM).

7. The method of claim 1, wherein the concentration of the non-ionic surfactant is 0.005% to 0.1%.

8. The method of claim 7, wherein the concentration is 0.01% to 0.02%.

9. The method of claim 8, wherein the concentration is 0.015%.

10. The method of claim 1, wherein the method results in at least about a 20-fold enhancement in the mass spectrometry signal from the sample when compared to a sample not treated with the non-ionic surfactant.

11. The method of claim 1, wherein the detected protein comprises an amino acid sequence of a peptide of Tables 2-7.

12. A method for performing proteomic analysis on a single cell, the method comprising isolating a single cell to prepare a sample, treating the sample with a non-ionic surfactant, performing mass spectrometry on the treated sample and detecting proteins in the treated sample.

13. The method of claim 12, wherein the non-ionic surfactant is an alkyl glucoside.

14. The method of claim 12, wherein the non-ionic surfactant is an alkyl diglucoside.

15. The method of claim 12, wherein the non-ionic surfactant is an alkyl maltoside.

16. The method of claim 12, wherein the non-ionic surfactant is octyl-maltoside, decyl-maltoside, dodecyl-maltoside, or tetradecyl-maltoside.

17. The method of claim 12, wherein the non-ionic surfactant is n-dodecyl-β-D-maltoside (DDM).

18. The method of claim 12, wherein the concentration of the non-ionic surfactant is 0.005% to 0.1%.

19. The method of claim 18, wherein the concentration is 0.01% to 0.02%.

20. The method of claim 19, wherein the concentration is 0.015%.

21. The method of claim 12, wherein the method results in at least about a 20-fold enhancement in the mass spectrometry signal from the sample when compared to a sample not treated with the non-ionic surfactant.

22. The method of claim 1, wherein the detected protein comprises an amino acid sequence of a peptide of Tables 2-7.


SEQUENCE LISTING (199 sequences)

SEQ ID NO: 1 - protein, 11 aa, Artificial Sequence
  Synthetic
LVVVGADGVGK

SEQ ID NO: 2 - protein, 11 aa, Artificial Sequence
  Synthetic
LVVVGAGGVGK

SEQ ID NO: 3 - protein, 13 aa, Artificial Sequence
  Synthetic
FVSGVLSDQMSAR

SEQ ID NO: 4 - protein, 29 aa, Artificial Sequence
  Synthetic
VNPKGLDEESKDYLSLYLLLVSCPKSEVR

SEQ ID NO: 5 - protein, 29 aa, Artificial Sequence
  Synthetic
VNPKGLDEESKDYLSLNLLLVSCPKSEVR

SEQ ID NO: 6 - protein, 22 aa, Artificial Sequence
  Synthetic
AKFKFSILNAKGEETKAMESQR

SEQ ID NO: 7 - protein, 22 aa, Artificial Sequence
  Synthetic
AKCKFSILNAKGEETKAMESQR

SEQ ID NO: 8 - DNA, 47 bp, Artificial Sequence
  Synthetic- linker
ggagatctaggaggtggaggtagcggtggaggtggaagccaggatcc

SEQ ID NO: 9 - protein, 10 aa, Artificial Sequence
  Synthetic
AENGLLMTPC

SEQ ID NO: 10 - protein, 11 aa, Artificial Sequence
  Synthetic
TANFVAPEVLK

SEQ ID NO: 11 - protein, 10 aa, Artificial Sequence
  Synthetic
EGSVLDILKS

SEQ ID NO: 12 - protein, 7 aa, Artificial Sequence
  Synthetic
PGFASPK

SEQ ID NO: 13 - protein, 7 aa, Artificial Sequence
  Synthetic
ELVEPLT

SEQ ID NO: 14 - protein, 12 aa, Artificial Sequence
  Synthetic
PSGEAPNQALLR

SEQ ID NO: 15 - protein, 10 aa, Artificial Sequence
  Synthetic
WIQEINVAEK

SEQ ID NO: 16 - protein, 6 aa, Artificial Sequence
  Synthetic
GSHQIS

SEQ ID NO: 17 - protein, 13 aa, Artificial Sequence
  Synthetic
LDNPDYQQDFFPK

SEQ ID NO: 18 - protein, 15 aa, Artificial Sequence
  Synthetic
NNTVIDELPFKSPIT

SEQ ID NO: 19 - protein, 7 aa, Artificial Sequence
  Synthetic
QLVHMVK

SEQ ID NO: 20 - protein, 7 aa, Artificial Sequence
  Synthetic
QLVHVVK

SEQ ID NO: 21 - protein, 12 aa, Artificial Sequence
  Synthetic
TDAEFVCEWTLK

SEQ ID NO: 22 - protein, 9 aa, Artificial Sequence
  Synthetic
TDAEFVCER

SEQ ID NO: 23 - protein, 7 aa, Artificial Sequence
  Synthetic
CDTEIDR

SEQ ID NO: 24 - protein, 7 aa, Artificial Sequence
  Synthetic
YDTEIDR

SEQ ID NO: 25 - protein, 12 aa, Artificial Sequence
  Synthetic
TSSGLYIFCNER

SEQ ID NO: 26 - protein, 9 aa, Artificial Sequence
  Synthetic
TSSGLYIFR

SEQ ID NO: 27 - protein, 6 aa, Artificial Sequence
  Synthetic
LTVDQK

SEQ ID NO: 28 - protein, 6 aa, Artificial Sequence
  Synthetic
LTVGQK

SEQ ID NO: 29 - protein, 10 aa, Artificial Sequence
  Synthetic
LMVPFAMVIR

SEQ ID NO: 30 - protein, 10 aa, Artificial Sequence
  Synthetic
WMVPFAMVIR

SEQ ID NO: 31 - protein, 18 aa, Artificial Sequence
  Synthetic
ADQQYECGAEIGEGAYGK

SEQ ID NO: 32 - protein, 18 aa, Artificial Sequence
  Synthetic
ADQQYECVAEIGEGAYGK

SEQ ID NO: 33 - protein, 9 aa, Artificial Sequence
  Synthetic
NVSFEDAGK

SEQ ID NO: 34 - protein, 36 aa, Artificial Sequence
  Synthetic
NVSFEDAGEYTCLAGNSIGLSHHSAWLTVLEALEER

SEQ ID NO: 35 - protein, 11 aa, Artificial Sequence
  Synthetic
NSLFNPAALSR

SEQ ID NO: 36 - protein, 11 aa, Artificial Sequence
  Synthetic
NSSFNPAALSR

SEQ ID NO: 37 - protein, 6 aa, Artificial Sequence
  Synthetic
VHDSLK

SEQ ID NO: 38 - protein, 10 aa, Artificial Sequence
  Synthetic
VHDSLEDFPK

SEQ ID NO: 39 - protein, 18 aa, Artificial Sequence
  Synthetic
VAQYPFENHNPPQLELIK

SEQ ID NO: 40 - protein, 18 aa, Artificial Sequence
  Synthetic
VAQYPFEDHNPPQLELIK

SEQ ID NO: 41 - protein, 23 aa, Artificial Sequence
  Synthetic
PFCEDLDQWLSEDDNHVAAIYCK

SEQ ID NO: 42 - protein, 23 aa, Artificial Sequence
  Synthetic
PFCEDLDQWLSEDDNHVAAIHCK

SEQ ID NO: 43 - protein, 18 aa, Artificial Sequence
  Synthetic
IMESLAWLSDSPLCDLIK

SEQ ID NO: 44 - protein, 18 aa, Artificial Sequence
  Synthetic
IMESLAWLSDSPLFDLIK

SEQ ID NO: 45 - protein, 15 aa, Artificial Sequence
  Synthetic
LPFDIEGSDEADGSK

SEQ ID NO: 46 - protein, 7 aa, Artificial Sequence
  Synthetic
LVENIGK

SEQ ID NO: 47 - protein, 7 aa, Artificial Sequence
  Synthetic
LFENIGK

SEQ ID NO: 48 - protein, 23 aa, Artificial Sequence
  Synthetic
CWGESSEDCQSLTHTVCAGGCAR

SEQ ID NO: 49 - protein, 14 aa, Artificial Sequence
  Synthetic
CWGESSEDCQSLTR

SEQ ID NO: 50 - protein, 23 aa, Artificial Sequence
  Synthetic
CWGESSEDCQSLTCTVCAGGCAR

SEQ ID NO: 51 - protein, 14 aa, Artificial Sequence
  Synthetic
CWGESSEDCQSLTR

SEQ ID NO: 52 - protein, 42 aa, Artificial Sequence
  Synthetic
YTFGASCVTACPYNYLSTDVGFCTLVCPLHNQEVTAEDGTQR

SEQ ID NO: 53 - protein, 42 aa, Artificial Sequence
  Synthetic
YTFGASCVTACPYNYLSTDVGSCTLVCPLHNQEVTAEDGTQR

SEQ ID NO: 54 - protein, 42 aa, Artificial Sequence
  Synthetic
YTFGASCVTACPYNYLSTDVGYCTLVCPLHNQEVTAEDGTQR

SEQ ID NO: 55 - protein, 24 aa, Artificial Sequence
  Synthetic
LLQETELVEPLTPSGAMPNLAQMR

SEQ ID NO: 56 - protein, 24 aa, Artificial Sequence
  Synthetic
LLQETELVEPLTPSGAMPNQAQMR

SEQ ID NO: 57 - protein, 19 aa, Artificial Sequence
  Synthetic
EMLDEAYVMAGVGSPYVSR

SEQ ID NO: 58 - protein, 19 aa, Artificial Sequence
  Synthetic
EILDEAYVMAGVGSPYVSR

SEQ ID NO: 59 - protein, 31 aa, Artificial Sequence
  Synthetic
VPGFVDLTLHDQVHLLQCAWLEILMIGLVWR

SEQ ID NO: 60 - protein, 31 aa, Artificial Sequence
  Synthetic
VPGFVDLTLHDQVHLLECAWLEILMIGLVWR

SEQ ID NO: 61 - protein, 18 aa, Artificial Sequence
  Synthetic
SIILLNSGVYTFLPSTLK

SEQ ID NO: 62 - protein, 18 aa, Artificial Sequence
  Synthetic
SIILLNSGVYTFLSSTLK

SEQ ID NO: 63 - protein, 11 aa, Artificial Sequence
  Synthetic
ITDTLMHLMAK

SEQ ID NO: 64 - protein, 11 aa, Artificial Sequence
  Synthetic
ITDTLIHLMAK

SEQ ID NO: 65 - protein, 17 aa, Artificial Sequence
  Synthetic
NVVPPYDLLLEMLDAHR

SEQ ID NO: 66 - protein, 17 aa, Artificial Sequence
  Synthetic
NVVPLYDLLLEMLDAHR

SEQ ID NO: 67 - protein, 5 aa, Artificial Sequence
  Synthetic
NVVPR

SEQ ID NO: 68 - protein, 17 aa, Artificial Sequence
  Synthetic
NVVPHYDLLLEMLDAHR

SEQ ID NO: 69 - protein, 17 aa, Artificial Sequence
  Synthetic
NVVPLSDLLLEMLDAHR

SEQ ID NO: 70 - protein, 17 aa, Artificial Sequence
  Synthetic
NVVPLNDLLLEMLDAHR

SEQ ID NO: 71 - protein, 17 aa, Artificial Sequence
  Synthetic
NVVPLCDLLLEMLDAHR

SEQ ID NO: 72 - protein, 17 aa, Artificial Sequence
  Synthetic
NVVPLDDLLLEMLDAHR

SEQ ID NO: 73 - protein, 17 aa, Artificial Sequence
  Synthetic
NVVPLGDLLLEMLDAHR

SEQ ID NO: 74 - protein, 17 aa, Artificial Sequence
  Synthetic
NVVPLHDLLLEMLDAHR

SEQ ID NO: 75 - protein, 17 aa, Artificial Sequence
  Synthetic
NVVPLYGLLLEMLDAHR

SEQ ID NO: 76 - protein, 8 aa, Artificial Sequence
  Synthetic
EAFFDETR

SEQ ID NO: 77 - protein, 8 aa, Artificial Sequence
  Synthetic
EEFFDETR

SEQ ID NO: 78 - protein, 6 aa, Artificial Sequence
  Synthetic
QLCDLR

SEQ ID NO: 79 - protein, 5 aa, Artificial Sequence
  Synthetic
LCDLR

SEQ ID NO: 80 - protein, 12 aa, Artificial Sequence
  Synthetic
LCDLQLFQPFLK

SEQ ID NO: 81 - protein, 8 aa, Artificial Sequence
  Synthetic
VIEPVVNR

SEQ ID NO: 82 - protein, 8 aa, Artificial Sequence
  Synthetic
VIEPVGNR

SEQ ID NO: 83 - protein, 8 aa, Artificial Sequence
  Synthetic
VIEPVGTR

SEQ ID NO: 84 - protein, 11 aa, Artificial Sequence
  Synthetic
VIEPVGNHEEK

SEQ ID NO: 85 - protein, 17 aa, Artificial Sequence
  Synthetic
EIDFAIGMPVCEFDMVK

SEQ ID NO: 86 - protein, 17 aa, Artificial Sequence
  Synthetic
EIGFAIGMPVCEFDMVK

SEQ ID NO: 87 - protein, 8 aa, Artificial Sequence
  Synthetic
ILCATYVK

SEQ ID NO: 88 - protein, 12 aa, Artificial Sequence
  Synthetic
ILCATYVNVNIR

SEQ ID NO: 89 - protein, 8 aa, Artificial Sequence
  Synthetic
TGIYHGGK

SEQ ID NO: 90 - protein, 18 aa, Artificial Sequence
  Synthetic
TGIYHGGEPLCDNVNTQR

SEQ ID NO: 91 - protein, 4 aa, Artificial Sequence
  Synthetic
EEHR

SEQ ID NO: 92 - protein, 24 aa, Artificial Sequence
  Synthetic
EEHCPLAWGNINLFDYTDTLVSGK

SEQ ID NO: 93 - protein, 13 aa, Artificial Sequence
  Synthetic
MALNLWPVPHGLK

SEQ ID NO: 94 - protein, 28 aa, Artificial Sequence
  Synthetic
MALNLWPVPHGLEDLLNPIGVTGSNPNK

SEQ ID NO: 95 - protein, 5 aa, Artificial Sequence
  Synthetic
DPLSK

SEQ ID NO: 96 - protein, 11 aa, Artificial Sequence
  Synthetic
DPLSEITEQEK

SEQ ID NO: 97 - protein, 11 aa, Artificial Sequence
  Synthetic
DPLSQITEQEK

SEQ ID NO: 98 - protein, 8 aa, Artificial Sequence
  Synthetic
DPLSEITK

SEQ ID NO: 99 - protein, 11 aa, Artificial Sequence
  Synthetic
DPLSEITQQEK

SEQ ID NO: 100 - protein, 11 aa, Artificial Sequence
  Synthetic
DPLSEITGQEK

SEQ ID NO: 101 - protein, 9 aa, Artificial Sequence
  Synthetic
DPLSEITER

SEQ ID NO: 102 - protein, 9 aa, Artificial Sequence
  Synthetic
DPLSEITEK

SEQ ID NO: 103 - protein, 5 aa, Artificial Sequence
  Synthetic
DETQK

SEQ ID NO: 104 - protein, 17 aa, Artificial Sequence
  Synthetic
SCAGYCVATFILGIEDR

SEQ ID NO: 105 - protein, 17 aa, Artificial Sequence
  Synthetic
SCAGYCVATFILGIGDR

SEQ ID NO: 106 - protein, 6 aa, Artificial Sequence
  Synthetic
SLALDK

SEQ ID NO: 107 - protein, 6 aa, Artificial Sequence
  Synthetic
TLALDK

SEQ ID NO: 108 - protein, 6 aa, Artificial Sequence
  Synthetic
TLALNK

SEQ ID NO: 109 - protein, 7 aa, Artificial Sequence
  Synthetic
TEQEALK

SEQ ID NO: 110 - protein, 11 aa, Artificial Sequence
  Synthetic
TEQEALEYFMK

SEQ ID NO: 111 - protein, 6 aa, Artificial Sequence
  Synthetic
QMNDAR

SEQ ID NO: 112 - protein, 13 aa, Artificial Sequence
  Synthetic
QMNDAHHGGWTTK

SEQ ID NO: 113 - protein, 13 aa, Artificial Sequence
  Synthetic
QMNDALHGGWTTK

SEQ ID NO: 114 - protein, 13 aa, Artificial Sequence
  Synthetic
QMNDAQHGGWTTK

SEQ ID NO: 115 - protein, 8 aa, Artificial Sequence
  Synthetic
QMNDAHHR

SEQ ID NO: 116 - protein, 12 aa, Artificial Sequence
  Synthetic
SVTCTNSPALNK

SEQ ID NO: 117 - protein, 12 aa, Artificial Sequence
  Synthetic
SVTCTYSPALNK

SEQ ID NO: 118 - protein, 12 aa, Artificial Sequence
  Synthetic
SVTCTYSPALNR

SEQ ID NO: 119 - protein, 19 aa, Artificial Sequence
  Synthetic
SVTCTYSPALNEMFCQLAK

SEQ ID NO: 120 - protein, 7 aa, Artificial Sequence
  Synthetic
MFYQLAK

SEQ ID NO: 121 - protein, 7 aa, Artificial Sequence
  Synthetic
MFCQLAK

SEQ ID NO: 122 - protein, 17 aa, Artificial Sequence
  Synthetic
TCPMQLWVDSTPPPGTR

SEQ ID NO: 123 - protein, 17 aa, Artificial Sequence
  Synthetic
TCPVQLWVDSTPPPGTR

SEQ ID NO: 124 - protein, 10 aa, Artificial Sequence
  Synthetic
QSQHMTEFVR

SEQ ID NO: 125 - protein, 10 aa, Artificial Sequence
  Synthetic
QSQHMTEVVR

SEQ ID NO: 126 - protein, 10 aa, Artificial Sequence
  Synthetic
QSQHMTEVMR

SEQ ID NO: 127 - protein, 15 aa, Artificial Sequence
  Synthetic
CSDSDGLAPPQLLIR

SEQ ID NO: 128 - protein, 15 aa, Artificial Sequence
  Synthetic
CSDSDGLAPPQHLIR

SEQ ID NO: 129 - protein, 19 aa, Artificial Sequence
  Synthetic
SPILTIITLEDSSGNLLGR

SEQ ID NO: 130 - protein, 19 aa, Artificial Sequence
  Synthetic
RPILTIITLEDSSGNLLGR

SEQ ID NO: 131 - protein, 6 aa, Artificial Sequence
  Synthetic
NSLEVR

SEQ ID NO: 132 - protein, 6 aa, Artificial Sequence
  Synthetic
NSFEVR

SEQ ID NO: 133 - protein, 13 aa, Artificial Sequence
  Synthetic
NSFEVHVCACPGR

SEQ ID NO: 134 - protein, 13 aa, Artificial Sequence
  Synthetic
NSFEVCVCACPGR

SEQ ID NO: 135 - protein, 9 aa, Artificial Sequence
  Synthetic
VCACPGGDR

SEQ ID NO: 136 - protein, 7 aa, Artificial Sequence
  Synthetic
VCACPGR

SEQ ID NO: 137 - protein, 7 aa, Artificial Sequence
  Synthetic
TEVENLR

SEQ ID NO: 138 - protein, 7 aa, Artificial Sequence
  Synthetic
TEEENLR

SEQ ID NO: 139 - protein, 11 aa, Artificial Sequence
  Synthetic
LVVVGAGGVGK

SEQ ID NO: 140 - protein, 11 aa, Artificial Sequence
  Synthetic
LVVVGAVGVGK

SEQ ID NO: 141 - protein, 11 aa, Artificial Sequence
  Synthetic
LVVVGACGVGK

SEQ ID NO: 142 - protein, 11 aa, Artificial Sequence
  Synthetic
LVVVGAAGVGK

SEQ ID NO: 143 - protein, 7 aa, Artificial Sequence
  Synthetic
LVVVGAR

SEQ ID NO: 144 - protein, 11 aa, Artificial Sequence
  Synthetic
LVVVGAGVVGK

SEQ ID NO: 145 - protein, 11 aa, Artificial Sequence
  Synthetic
LVVVGAGGVGK

SEQ ID NO: 146 - protein, 11 aa, Artificial Sequence
  Synthetic
LVVVGAGGIGK

SEQ ID NO: 147 - protein, 26 aa, Artificial Sequence
  Synthetic
QVVIDGETCLLDILDTAGHEEYSAMR

SEQ ID NO: 148 - protein, 26 aa, Artificial Sequence
  Synthetic
QVVIDGETCLLDILDTAGQEEYSAMR

SEQ ID NO: 149 - protein, 19 aa, Artificial Sequence
  Synthetic
DSEDVPMVLVGNNCDLPSR

SEQ ID NO: 150 - protein, 13 aa, Artificial Sequence
  Synthetic
DSEDVPMVLVGNK

SEQ ID NO: 151 - protein, 12 aa, Artificial Sequence
  Synthetic
SYGIPFIETSTK

SEQ ID NO: 152 - protein, 12 aa, Artificial Sequence
  Synthetic
SYGIPFIETSAK

SEQ ID NO: 153 - protein, 27 aa, Artificial Sequence
  Synthetic
LLGICLTSTVQLIMQLMPFGCLLDYVR

SEQ ID NO: 154 - protein, 27 aa, Artificial Sequence
  Synthetic
LLGICLTSTVQLITQLMPFGCLLDYVR

SEQ ID NO: 155 - protein, 6 aa, Artificial Sequence
  Synthetic
ITDFGR

SEQ ID NO: 156 - protein, 8 aa, Artificial Sequence
  Synthetic
ITDFGLAK

SEQ ID NO: 157 - protein, 19 aa, Artificial Sequence
  Synthetic
EILHEAYVMAGVGSPYVSR

SEQ ID NO: 158 - protein, 19 aa, Artificial Sequence
  Synthetic
EILYEAYVMAGVGSPYVSR

SEQ ID NO: 159 - protein, 19 aa, Artificial Sequence
  Synthetic
EILDEAYVMAGLGSPYVSR

SEQ ID NO: 160 - protein, 28 aa, Artificial Sequence
  Synthetic
MALNLWPVPHVLEDLLNPIGVTGSNPNK

SEQ ID NO: 161 - protein, 28 aa, Artificial Sequence
  Synthetic
MALNLWPVPHGLQDLLNPIGVTGSNPNK

SEQ ID NO: 162 - protein, 28 aa, Artificial Sequence
  Synthetic
MALNLWPVPHGLGDLLNPIGVTGSNPNK

SEQ ID NO: 163 - protein, 31 aa, Artificial Sequence
  Synthetic
QHANLFINLFSIMLGSGMPELQSFDDIAYIR

SEQ ID NO: 164 - protein, 31 aa, Artificial Sequence
  Synthetic
QHANLFINLFSMMLGSGMPELQSFDDIAYIR

SEQ ID NO: 165 - protein, 75 aa, Artificial Sequence
  Synthetic
LSPLTIVSWLNVYMQVAYLNDLHEVLLPQYPQQIFIQIAELLDLCVLDVDCLEFPHGILAASALYHFSSSELMQK

SEQ ID NO: 166 - protein, 75 aa, Artificial Sequence
  Synthetic
LSPLTIVSWLNVYMQVAYLNDLHEVLLPQYPQQIFIQIAELLDLCVLDVDCLEFPYGILAASALYHFSSSELMQK

SEQ ID NO: 167 - protein, 5 aa, Artificial Sequence
  Synthetic
DTELR

SEQ ID NO: 168 - protein, 15 aa, Artificial Sequence
  Synthetic
LLDIDETEYHADGGK

SEQ ID NO: 169 - protein, 27 aa, Artificial Sequence
  Synthetic
ILVECLLPNGMIVTLECLHEATLITIK

SEQ ID NO: 170 - protein, 19 aa, Artificial Sequence
  Synthetic
ILVECLLPNGMIVTLECLR

SEQ ID NO: 171 - protein, 31 aa, Artificial Sequence; identical to SEQ ID NO: 164
  Synthetic

SEQ ID NO: 172 - protein, 17 aa, Artificial Sequence
  Synthetic
TCPVQLWVDSTSPPGTR

SEQ ID NO: 173 - protein, 17 aa, Artificial Sequence
  Synthetic
TCPVQLWVDSTHPPGTR

SEQ ID NO: 174 - protein, 17 aa, Artificial Sequence
  Synthetic
TCPVQLWVDSTPLPGTR

SEQ ID NO: 175 - protein, 17 aa, Artificial Sequence
  Synthetic
TCPVQLWVDSTPPPGIR

SEQ ID NO: 176 - protein, 19 aa, Artificial Sequence
  Synthetic
TCPVQLWVDSTPPPGTPVR

SEQ ID NO: 177 - protein, 7 aa, Artificial Sequence
  Synthetic
HCPHHER

SEQ ID NO: 178 - protein, 4 aa, Artificial Sequence
  Synthetic
CPHR

SEQ ID NO: 179 - protein, 6 aa, Artificial Sequence
  Synthetic
CPHHER

SEQ ID NO: 180 - protein, 6 aa, Artificial Sequence
  Synthetic
CPHYER

SEQ ID NO: 181 - protein, 35 aa, Artificial Sequence
  Synthetic
HSVVVPYEPPEVGSDCTTIHYNYMCNSSCMGGMNR

SEQ ID NO: 182 - protein, 35 aa, Artificial Sequence
  Synthetic
HSVVVSYEPPEVGSDCTTIHYNYMCNSSCMGGMNR

SEQ ID NO: 183 - protein, 35 aa, Artificial Sequence
  Synthetic
HSVVVPCEPPEVGSDCTTIHYNYMCNSSCMGGMNR

SEQ ID NO: 184 - protein, 35 aa, Artificial Sequence
  Synthetic
HSVVVPYEPPEVGSDCTTIHYNYMSNSSCMGGMNR

SEQ ID NO: 185 - protein, 35 aa, Artificial Sequence
  Synthetic
HSVVVPYEPPEVGSDCTTIHYNYMFNSSCMGGMNR

SEQ ID NO: 186 - protein, 35 aa, Artificial Sequence
  Synthetic
HSVVVPYEPPEVGSDCTTIHYNYMYNSSCMGGMNR

SEQ ID NO: 187 - protein, 35 aa, Artificial Sequence
  Synthetic
HSVVVPYEPPEVGSDCTTIHYNYMCNSSFMGGMNR

SEQ ID NO: 188 - protein, 35 aa, Artificial Sequence
  Synthetic
HSVVVPYEPPEVGSDCTTIHYNYMCNSSCMDGMNR

SEQ ID NO: 189 - protein, 35 aa, Artificial Sequence
  Synthetic
HSVVVPYEPPEVGSDCTTIHYNYMCNSSCMGSMNR

SEQ ID NO: 190 - protein, 35 aa, Artificial Sequence
  Synthetic
HSVVVPYEPPEVGSDCTTIHYNYMCNSSCMGGVNR

SEQ ID NO: 191 - protein, 36 aa, Artificial Sequence
  Synthetic
HSVVVPYEPPEVGSDCTTIHYNYMCNSSCMGGMNWR

SEQ ID NO: 192 - protein, 36 aa, Artificial Sequence
  Synthetic
HSVVVPYEPPEVGSDCTTIHYNYMCNSSCMGGMNQR

SEQ ID NO: 193 - protein, 5 aa, Artificial Sequence
  Synthetic
VCACR

SEQ ID NO: 194 - protein, 13 aa, Artificial Sequence
  Synthetic
IADPEHDHTGFLT

SEQ ID NO: 195 - protein, 4 aa, Artificial Sequence
  Synthetic
VATR

SEQ ID NO: 196 - protein, 6 aa, Artificial Sequence
  Synthetic
NGLQSC

SEQ ID NO: 197 - protein, 4 aa, Artificial Sequence
  Synthetic
FLQR

SEQ ID NO: 198 - protein, 18 aa, Artificial Sequence
  Synthetic
GGGDLTLGLEPSEEEAPR

SEQ ID NO: 199 - protein, 6 aa, Artificial Sequence
  Synthetic
PIKEDS